(19) United States
(12) Patent Application Publication
(10) Pub. No.: US 2024/0366696 A1
(43) Pub. Date: Nov. 7, 2024

(54) USING REPROGRAMMED, PERSONALIZED CANCER STEM CELLS AS A TRIPLE-KILLER CANCER TREATMENT

(71) Applicant: CALIFORNIA NORTHSTATE COLLEGE OF PHARMACY, LLC, ELK GROVE, CA (US)

(72) Inventors: DIPONGKOR SAHA, ELK GROVE, CA (US); XIAODONG FENG, ELK GROVE, CA (US); FAKHRUL AHSAN, ELK GROVE, CA (US)

(21) Appl. No.: 18/315,782

(22) Filed: May 11, 2023

Related U.S. Application Data
(60) Provisional application No. 63/462,908, filed on Apr. 28, 2023.

(51) Int. Cl. A61K 35/763; A61P 35/00; C12N 5/095

(52) U.S. Cl. CPC A61K 35/763; A61P 35/00; C12N 5/0695; C12N 2510/04

(57) ABSTRACT

A “triple-killer” cancer treatment is provided. Live, cancer stem cells carrying an oncolytic virus expressing an immunomodulator are re-programmed to die within a desired time frame after administration to a subject. The cancer treatment (1) releases a cancer antigen cocktail that creates cancer antibodies in the subject, (2) kills cancer tumor cells in the subject through release of the oncolytic virus itself, and (3) kills additional cancer tumor cells in the subject through the expression of the immunomodulator, the treatment further providing an immunological memory response. The oncolytic virus is amplified in the cancer stem cell itself, before, during, and after the administration, avoiding the toxicity associated with a pure administration of the immunomodulator. The oncolytic virus is constructed to selectively replicate in the tumor/tumors stem cells of the subject, resulting in their death and release of tumor antigens to augment immune response in the triple-killer treatment.

### CROSS-REFERENCE TO RELATED APPLICATIONS

This application claims the benefit of U.S. Provisional Application No. 63/462,908, filed Apr. 28, 2023, which is hereby incorporated herein by reference in its entirety.

### SEQUENCE LISTINGS

The instant application contains a sequence listing which has been submitted in XML format via EFS-Web, which is hereby incorporated by reference in its entirety. The XML copy, created on May 4, 2023, is named CALPP007US01_SL.xml and is 191,801 bytes in size.

### BACKGROUND

### Field of the Invention

The teachings are directed to cancer treatments that administer live, cancer stem cells that are personalized and re-programmed to die in a desired time-frame with an oncolytic virus that kills the cancer stem cell, releases antigens, and expresses an immunomodulatory cytokine, the treatment providing a sustained immunological memory response.

### Description of the Related Art

The current standard-of-care for treatment of cancer includes surgical resection, radiation therapy, chemotherapy, targeted therapies, immunotherapy, and the like. There are other therapies in development, such as nanotechnology-based innovations, photodynamic strategies, gene therapy, and local destruction of the tumors using genetically modified bacteria or controlled hyperthermia, although they're all suffering in a showing of long-term benefit.

Recently, anticancer vaccines have gained attention in boosting antitumor immunity and targeting/killing tumor cells or cancer stem cells. In these developing technologies, lysates of tumor cells or cancer stem cells, or any tumor-derived antigens (such as EGFRvIII, heat shock protein, are used to pulse dendritic cells, which are antigen presenting cells in the body's immune system. The pulsed dendritic cells are delivered to patients to incite immune responses against cancer. Overall, the use of dendritic cells is inconvenient due to expense and difficulty to culture the cells for delivery. The EGFRvIII vaccine (Rindopepimut), for example, was discontinued in early 2016 for failing to significantly improve survival, emphasizing the need for alternate and newer vaccine-based strategies.

Glioblastoma was the cancer of choice to test the cancer treatment methods taught herein, as it is a devastating disease that needs a new method of treatment. Glioblastoma is the most common form of primary malignant brain tumor, is almost always lethal with current treatments, and has an average patient survival of about 12-15 months. One of the major factors that contribute to treatment failure is the presence of cancer stem cells, a subpopulation of tumor cells responsible for tumor recurrence and resistance to therapy, such as radiation and chemotherapy. Cancer stem cells also induce immunosuppression which confounds immunotherapy. Due to all these factors, cancer stem cells are considered critical therapeutic targets. As such, a killing of cancer stem cells can inhibit the production of new cancer cells and decrease resistance of the cancer cells to therapies. Moreover, since few systemic treatments can get through the blood-brain barrier, another cause of treatment failure is the inability to get through the blood-brain barrier to treat brain tumors.

Cancer stem cells represent about 1-3% of all the cancer cells and can regenerate the new growth of cancer with potential for new antigens and hallmark features that allow for escaping the immune system and existing cancer treatments. Uke cancer cells, stem cells can show unlimited proliferation. However, the proliferation of stem cells can be under control while cancer cell proliferation is out of control. The technology provided herein allows isolation and culture of cancer specific stem cells in vitro to generate a cancer vaccine having a cocktail of all the potential antigens essential for cancer growth. The cancer antigen cocktail consists of antigens already presented by matured tumor cells, but the stem cells also provide many unknown cancer antigens that might help the cancer to escape the immune attack, and escape target therapy, immunotherapy or chemotherapy. One of skill in the art will appreciate an anticancer vaccine that (i) provides 100% protection against the development of a cancer, (ii) provides a long-term survival, (iii) does not require the use of dendritic cells, (iv) gets through the blood-brain barrier to treat brain tumors, (v) shows no obvious toxicity, and (vi) acts as a triple-killer, releasing 3 therapeutic attacks that include
1) death to the cancer through the cancer antigen cocktail;
2) death to the cancer through the oncolytic virus; and,
3) death to the cancer through essential immune cytokines;
   wherein, the triple-killer also provides a sustained immunological memory response to provide a sustained death to the cancer. Since the oncolytic virus is amplified in the cancer stem cell itself, this provides a protective packaging that avoids the toxicity associated with a pure administration of the immunomodulatory cytokine to the subject which is suffered by state-of-the-art IV, IM, or subcutaneous deliveries of the cytokines. The protective packaging that allows for production of the oncolytic virus while in the live cancer stem cell, less oncolytic virus can be administered when administered through cancer stem cells when compared to I.V. or I.M. administrations of the oncolytic virus itself. Likewise, less of the toxic immunomodulatory cytokine is administered through cancer stem cells when compared to I.V. or I.M. administrations of the cytokine itself. Since the oncolytic virus is selective to the cancer cells, the therapies are further targeted. The cytokine will only be expressed locally in the cancer microenvironment where there is a presence of the target cancer cells. This is because the oncolytic viruses will be eliminated from the body where there is an absence of the target cancer cells. For at least these reasons, the systems taught herein can also serve as great vaccine to not only inhibit cancers, but also as a targeted therapeutic to at least inhibit, if not prevent, the onset of cancers.

The art will appreciate that the personalized treatments taught herein can be quite valuable to any patient at risk of, or suffering cancer, and in particular to those having a significant family history of cancer, a high risk of genetic mutations, or the presence of early stage of cancers. And, importantly, the personalized triple-killer effect provides a powerful and effective therapy of later stage cancers while avoiding the toxicity problems currently suffered by the art.

### SUMMARY OF THE INVENTION

The teachings herein are directed to a personalized, “triple-killer” cancer treatment that administers live, cancer stem cells that are re-programmed with an oncolytic virus that expresses an immunomodulatory cytokine, wherein the cancer treatment (1) kills the cancer stem cell after administration in a subject to release a cancer antigen cocktail that creates cancer antibodies in the subject, (2) kills cancer tumor cells in the subject through release of the oncolytic virus itself, and (3) kills additional cancer tumor cells in the subject, the immunomodulatory cytokine further providing an immunological memory response. Moreover, the oncolytic virus is amplified in the cancer stem cell itself, avoiding the toxicity associated with a pure administration of the immunomodulatory cytokine. Less oncolytic virus can be administered when administered through cancer stem cells when compared to I.V. or I.M. administrations of the oncolytic virus itself. Likewise, less of the toxic immunomodulatory cytokine is administered through cancer stem cells when compared to I.V. or I.M. administrations of the cytokine itself.

In some embodiments, the teachings are directed to a method of creating a delivery system for cancer stem cell antigens and an oncolytic virus. The method can include obtaining live cancer stem cells, programming the live cancer stem cells to die in a desired amount of time, loading the live cancer stem cells with an oncolytic virus, administering the live cancer stem cells in an environment in which they will die and release antigens from the dead cancer stem cells and release the oncolytic virus.

In some embodiments, the teachings are directed to a method of creating a delivery system for cancer stem cell antigens and an oncolytic virus that expresses a desired protein. The method can include obtaining live cancer stem cells, programming the live cancer stem cells to die in a desired amount of time, loading the live cancer stem cells with an oncolytic virus that expresses a desired protein, administering the live cancer stem cells in an environment in which they will die and release antigens from the dead cancer stem cells, release the oncolytic virus that expresses the desired protein.

In some embodiments, the teachings are directed to a general method of creating a delivery system for cancer stem cell antigens and an oncolytic virus, the delivery system administered to a subject. The method can include obtaining live cancer stem cells, programming the live cancer stem cells to die in a desired amount of time, loading the live cancer stem cells with an oncolytic virus, administering the live cancer stem cells in an environment in the subject in which they will die and release antigens from the dead cancer stem cells and release the oncolytic virus.

In some embodiments, the teachings are directed to a general method of creating a delivery system for cancer stem cell antigens and an oncolytic virus that expresses a desired protein, the delivery system administered to a subject. The method can include obtaining live cancer stem cells, programming the live cancer stem cells to die in a desired amount of time, loading the live cancer stem cells with an oncolytic virus that expresses a desired protein, administering the live cancer stem cells in an environment in the subject in which they will die and release antigens from the dead cancer stem cells, release the oncolytic virus that expresses the desired protein.

In some embodiments, the teachings are directed to a method of treating a cancer in a subject. The methods can include obtaining a protective, antigenic drug delivery system comprising reprogrammed cancer stem cells hosting an oncolytic virus specific to the cancer stem cells; wherein, the delivery system is configured for protecting the oncolytic virus during a transport of the oncolytic virus in the subject to increase the half-life of the oncolytic virus during the transport when compared to an unprotected transport of the oncolytic virus in the subject; amplifying the amount of the oncolytic virus during the protected transport of the oncolytic virus in the subject; and, killing the cancer stem cells in a desired amount of time into the transport to (i) release the oncolytic virus in the subject from the dead cancer stem cells; (ii) initiate an antigenic immune response in the subject to antigens released from the dead cancer stem cells; and, (iii) continue replication of the virus in tumor cells of the cancer; wherein, the method includes administering the antigenic delivery system to the subject.

In some embodiments, the obtaining of the delivery system can include creating the protective, antigenic delivery system, the creating including obtaining the cancer stem cells; reprogramming the cancer stem cells to die in a desired time for a programmed release of the oncolytic virus and the antigens from the cancer stem cell in the subject; obtaining the oncolytic virus; and, loading the cancer stem cells with the oncolytic virus to create the protective, antigenic delivery system.

In some embodiments, the obtaining of the cancer stem cells can include harvesting the cancer stem cells from the subject.

In some embodiments, the reprogramming of the cancer stem cells to die in the desired time for the programmed release of the oncolytic virus and the antigens from the cancer stem cell in the subject can include exposing the cancer stem cells to radiation.

In some embodiments, the reprogramming of the cancer stem cells to die in the desired time for the programmed release of the oncolytic virus and the antigens from the cancer stem cell in the subject includes selecting the type of the oncolytic virus, the amount of the oncolytic virus, or a combination thereof, used in the loading of the cancer stem cells with the oncolytic virus.

In some embodiments, the obtaining of the oncolytic virus includes
creating the oncolytic virus from a wild-type virus selected from the group consisting of Herpes Simplex Virus (HSV), Newcastle Disease Virus (NDV), Vesicular Stomatitis Virus (VSV), Reovirus, Measles Virus, Retrovirus, Influenza Virus, Sindbis Virus, Vaccinia Virus, and Adenovirus.

In some embodiments, the obtaining of the oncolytic virus includes
creating the oncolytic virus from a wild-type virus selected from the group consisting of Herpes Simplex Virus (HSV), Newcastle Disease Virus (NDV), Vesicular Stomatitis Virus (VSV), Reovirus, Measles Virus, Retrovirus, Influenza Virus, Sindbis Virus, Vaccinia Virus, and Adenovirus;
and, the creating includes deleting undesired genes from the wild-type oncolytic virus to make the oncolytic virus tumor specific; and, inserting desired genes that express desired proteins.

As such, the teachings provide a protective, antigenic drug delivery system. The protective, antigenic drug delivery system can include reprogrammed cancer stem cells; and, an oncolytic virus specific to the cancer cells, including cancer stem cells; wherein, the delivery system is configured for
protecting the oncolytic virus during a transport of the oncolytic virus in the subject to increase the half-life of the oncolytic virus during the transport when compared to an unprotected transport of the oncolytic virus in the subject;
amplifying the amount of the oncolytic virus during the protected transport of the oncolytic virus in the subject; and,
killing the cancer stem cells in a desired amount of time into the transport to ((i) release the oncolytic virus in the subject from the dead cancer stem cells; (ii) initiate an antigenic immune response in the subject to antigens released from the dead cancer stem cells; and, (iii) continue replication of the virus in tumor cells of a cancer.

In some embodiments, the protective, antigenic drug delivery system is created using a process that includes
obtaining the cancer stem cells;
reprogramming the cancer stem cells to die in a desired time for a programmed release of the oncolytic virus and the antigens from the cancer stem cell in the subject;
obtaining the oncolytic virus; and,
loading the cancer stem cells with the oncolytic virus to create the protective, antigenic delivery system.

In some embodiments, the obtaining of the cancer stem cells includes harvesting the cancer stem cells from the subject.

In some embodiments, the reprogramming of the cancer stem cells to die in the desired time for the programmed release of the oncolytic virus and the antigens from the cancer stem cell in the subject includes exposing the cancer stem cells to radiation.

In some embodiments, the reprogramming of the cancer stem cells to die in the desired time for the programmed release of the oncolytic virus and the antigens from the cancer stem cell in the subject includes selecting the type of the oncolytic virus, the amount of the oncolytic virus, or a combination thereof, used in the loading of the cancer stem cells with the oncolytic virus.

In some embodiments, the obtaining of the oncolytic virus includes
creating the oncolytic virus from a wild-type virus selected from the group consisting of Herpes Simplex Virus (HSV), Newcastle Disease Virus (NDV), Vesicular Stomatitis Virus (VSV), Reovirus, Measles Virus, Retrovirus, Influenza Virus, Sindbis Virus, Vaccinia Virus, and Adenovirus.

In some embodiments, the obtaining of the oncolytic virus includes
creating the oncolytic virus from a wild-type virus selected from the group consisting of Herpes Simplex Virus (HSV), Newcastle Disease Virus (NDV), Vesicular Stomatitis Virus (VSV), Reovirus, Measles Virus, Retrovirus, Influenza Virus, Sindbis Virus, Vaccinia Virus, and Adenovirus;
and, the creating includes deleting undesired genes from the wild-type oncolytic virus to make the oncolytic virus tumor specific; and, inserting desired genes that express desired proteins.

It should be appreciated that the oncolytic virus can be constructed to express any protein desired. In some embodiments, the desired proteins are immunomodulators. In some embodiments, the desired proteins are cytokines. And, in some embodiments, the desired proteins are chemokines.

The vaccines taught herein can be used in methods of treating, inhibiting the onset of, or perhaps even preventing solid cancers or liquid cancers. As such, cancer vaccines for treating, inhibiting the onset of, or preventing a liquid cancer or a solid cancer in a subject are provided.

In some embodiments, a method of treating, or at least inhibiting the onset of, a solid cancer in a subject by administering live cancer stem cells is provided. In these embodiments, the method can include collecting live cancer stem cells from a solid cancer tissue from a donor; reprogramming the live cancer stem cells to create reprogrammed cancer stem cells that die in a programmed time-frame; administering the reprogrammed cancer stem cells to a recipient, the dying of the cancer stem cells occurring after administering the reprogrammed cancer stem cells to the recipient; and, boosting cancer immunity in the recipient. The boosting can include releasing cancer stem cell antigens in the recipient after the death of the reprogrammed cancer stem cells; and, stimulating the subject's immune system; wherein, the releasing and stimulating results in boosting the cancer immunity against the solid cancer. The incorporation, protection, and release of the oncolytic viruses, including those that express desired proteins, such as cytokines, chemokines, and any desired immunomodulatory, provides the triple-killer effect in the treatment of cancer.

In some embodiments, a method of treating, or at least inhibiting the onset of, a liquid cancer in a subject by administering live cancer stem cells is provided. In these embodiments, the method can include collecting live cancer stem cells from a liquid cancer tissue from a donor; reprogramming the live cancer stem cells to create reprogrammed cancer stem cells that die in a programmed time-frame; administering the reprogrammed cancer stem cells to a recipient, the dying of the cancer stem cells occurring after administering the reprogrammed cancer stem cells to the recipient; and, boosting cancer immunity in the recipient. The boosting can include releasing cancer stem cell antigens in the recipient after the death of the reprogrammed cancer stem cells; and, stimulating the subject's immune system; wherein, the releasing and stimulating results in boosting the cancer immunity against the liquid cancer. The incorporation, protection, and release of the oncolytic viruses, including those that express desired proteins, such as cytokines, chemokines, and any desired immunomodulatory, provides the triple-killer effect in the treatment of cancer.

In some embodiments, a cancer vaccine for treating, or at least inhibiting the onset of, a solid cancer in a subject is provided. In these embodiments, the vaccine can include a carrier; and, reprogrammed cancer stem cells created using a process including collecting live cancer stem cells from the solid cancer tissue from a donor; and, reprogramming the live cancer stem cells to die in a programmed time-frame; the reprogramming including treating the live cancer stem cells with an effective amount of an apoptosis inducing agent. In these embodiments, the reprogrammed cancer stem cells die after being administered to the recipient; and, the death of the reprogrammed cancer stem cells results in boosting cancer immunity in the recipient. The boosting can include releasing cancer stem cell antigens from the reprogrammed cancer stem cells; and, stimulating the subject's immune system; wherein, the releasing and stimulating resulting in boosting the cancer immunity against the solid cancer.

Likewise, in some embodiments, a cancer vaccine for treating, or at least inhibiting the onset of, a liquid cancer in a subject is provided. In these embodiments, the vaccine can include a carrier; and, reprogrammed cancer stem cells created using a process including collecting live cancer stem cells from the liquid cancer tissue from a donor; and, reprogramming the live cancer stem cells to die in a programmed time-frame; the reprogramming including treating the live cancer stem cells with an effective amount of an apoptosis inducing agent. In these embodiments, the reprogrammed cancer stem cells die after being administered to the recipient; and, the death of the reprogrammed cancer stem cells results in boosting cancer immunity in the recipient. The boosting can include releasing cancer stem cell antigens from the reprogrammed cancer stem cells; and, stimulating the subject's immune system; wherein, the releasing and stimulating resulting in boosting the cancer immunity against the liquid cancer.

One of skill will appreciate that the cancer stem cells that are included in the vaccine can include any stem cell from any cancer in nature, and that the stem cells can be collected from a solid cancer or a liquid cancer.

In some embodiments, the cancer is a solid cancer, and the cancer stem cell is collected from a glioblastoma tissue. In some embodiments, the cancer is a solid cancer, and the cancer stem cell is collected from a colorectal cancer tissue. In some embodiments, the cancer is a solid cancer, and the cancer stem cell is collected from a breast cancer, an ovarian cancer, or a prostate cancer. In some embodiments, the cancer is a solid cancer, and the cancer stem cell is collected from a melanoma skin cancer tissue. In some embodiments, the cancer is a solid cancer, and the cancer stem cell is collected from a non-melanoma skin cancer tissue. In some embodiments, the cancer is a solid cancer, and the cancer stem cell is collected from a small cell lung cancer tissue. In some embodiments, the cancer is a solid cancer, and the cancer stem cell is collected from a non-small cell lung cancer tissue. In some embodiments, the cancer is a solid cancer, and the cancer stem cell is collected from a liver cancer tissue.

In some embodiments, the cancer is a liquid cancer, and the cancer stem cell is collected from a leukemia. In some embodiments, the cancer is a liquid cancer, and the cancer stem cell is collected from a lymphoma. In some embodiments, the cancer is a liquid cancer, and the cancer stem cell is collected from a multiple myeloma.

### BRIEF DESCRIPTION OF THE DRAWINGS

FIGS. 1A and 1B are flowcharts showing a general method of using apoptotic stem cells as a cancer vaccine, according to some embodiments.

FIGS. 2A and 2B show how a cancer stem cell vaccine provided 100% protection from glioblastoma in an orthotopic glioblastoma stem-cell-derived, according to some embodiments.

FIGS. 3A and 3B is a repeat study that verifies that the 100% protection shown in the model is reproducible, according to some embodiments.

FIGS. 4A and 4B show that superiority of the cancer-stem-cell vaccine over a tumor-cell-derived vaccine, according to some embodiments.

FIGS. 5A-5C show that CD4+ T cells are integral to the effectiveness of the cancer stem cell vaccine, according to some embodiments.

FIG. 6 shows the stemness of the pure, cancer cancer stem cells, according to some embodiments.

FIG. 7 shows an analysis of brain tissue, namely that mice vaccinated with the cancer stem cell vaccine had a significantly smaller number of cancer stem cells in the brain tissue than mice that did not receive the vaccine, according to some embodiments.

FIGS. 8A and 8B illustrate the oncolytic virus process, and expand to a flowchart showing a general method of using cancer stem cells as a triple-killer cancer treatment, according to some embodiments.

FIGS. 9A-9E illustrate the construct of an oncolytic virus expressing IL-2 and mCherry, according to some embodiments.

FIGS. 10A-10G show that the oncolytic virus does infect a variety of breast cancer cells, according to some embodiments.

FIGS. 11A-11D illustrate that dendritic cells (DCs) produce cytokines and are susceptible to cytokine-mediated activation in response to treatment with a virus-infected cancer cell supernatant, according to some embodiments.

FIGS. 12A-12C show that the oncolytic virus expressing IL-12 increased the number of tumor-infiltrating T cells, namely CD3+, CD4+, and CD8+, according to some embodiments.

FIGS. 13A-13F illustrate how the administration of an oncolytic virus that expresses IL-2 increased T cell activity in tumors and spleens harvested from BALB/c mice having orthotopic mouse mammary tumors, including an increase in tumor infiltrating T cells, central memory T cells, and cytotoxic T cells from administration of the IL-2 expressing oncolytic virus to the mice, according to some embodiments.

FIGS. 14A and 14B show an increase in IFN-γ and number of effector memory T cells when splenocytes taken from protected mice were re-challenged by being co-cultured with EMT6 cells, according to some embodiments.

### DETAILED DESCRIPTION

The teachings herein are directed to uses of programmed cancer stem cells as a cancer treatment for a subject, namely a vaccine, a two-prong killer, and three-prong killer. The term “subject” and “patient” are used interchangeably and refer to an animal such as a mammal including, but not limited to, non-primates such as, for example, a cow, pig, horse, cat, dog, rabbit, rat and mouse; and primates such as, for example, a monkey or a human. In some embodiments, the terms “two-prong killer” and “dual killer” are interchangeable. In some embodiments, the terms “three-prong killer” and “triple killer” are interchangeable.

The systems and methods taught herein can be designed for vaccination, to deliver an oncolytic virus, or to deliver an oncolytic virus that expresses a desired protein. In some embodiments, the systems and methods taught herein can use cancer stem cells as a delivery vehicle for an oncolytic virus expressing an immunomodulator. The cancer stem cells are administered live, programmed to die in a desired time, and can be heterologous, in some embodiments, and homologous for a personalized medicine approach, in other embodiments. The approach is to eliminate cancer stem cells to minimize treatment resistance and maximize the effect of the cancer treatments taught herein. The treatments can include an antigenic effect from the cancer stem cells used in the delivery, an apoptotic effect from the oncolytic virus delivered by the cancer stem cells, and an immunomodulatory effect from the immunomodulator expressed by the oncolytic virus. Using cancer stem cells as a delivery vehicle has the effect of providing a cellular mechanism for amplification of the oncolytic virus during delivery, as well as for protection of the oncolytic virus from elimination by the subject's immune system during the delivery. This delivery vehicle also, importantly, shields the subject from the toxicity of the oncolytic virus, and protects the subject from the toxic effects experienced when delivering the oncolytic virus alone to the subject in the large amounts usually required due to the subject's immune response to the oncolytic virus. On the contrary, the systems and methods taught herein, do not require administering a large systemic dose of the oncolytic virus to the subject. For at least these reasons, the systems and methods taught herein can, as a result, reduce or eliminate the hard-to-predict, harmful toxic effects experienced by the subject when administering an oncolytic virus. Those of skill will appreciate having delivery systems, even personalized delivery systems, that use cancer stem cells to deliver an oncolytic virus to a subject, shield the virus from the subject to avoid elimination of the virus for a desired time, shield the subject from the virus to avoid toxicity for a desired time, and amplify the oncolytic virus during the desired time before delivery at the desired time. Moreover, the cancer stem cells provide an additional antigenic effect by dying at the desired time for the delivery.

In some embodiments, the cancer stem cells can also be referred to herein as “cancer stem cells”, “cancer-specific stem cells” “programmed cancer stem cells”, “programmed cancer specific stem cells”, “engineered cancer stem cells”, and combinations thereof. To avoid any confusion, any mention of the use of “cancer cell” or “stem cell” as a medium of carrying an oncolytic virus into a subject is intended to mean “cancer stem cell”. It should be appreciated that, since the cancer stem cells harvested can be of a specific type, they can be described and utilized as a “cancer-specific” antigenic agent and/or delivery system. This is true, even though in some embodiments they may not be limited to use in treating, preventing, or at least inhibiting the onset of, only that specific type of cancer. And, as noted, the cancer stem cells can be administered alone as a vaccine, a delivery vehicle for an antigen cocktail produced by the programmed cancer stem cells. In some embodiments, the cancer stem cells can also be used as a two-prong killer, a delivery vehicle for an oncolytic virus carried by the host cancer stem cells. And, in some embodiments, the cancer stem cells can be used as three-prong killer, a delivery vehicle for an oncolytic virus that expresses a desired protein. The term “cancer”, as used herein, can be used to refer to two things, in some embodiments, namely (1) cancer stem cells that were harvested from a specific cancer type and release an antigen cocktail for that specific cancer type, and (2) an oncolytic virus that selectively replicates in tumor cells. The term “oncolytic virus” can be used to refer to a virus that selectively kills tumor cells as compared to normal cells. In addition, the term can be used to refer to a virus that is engineered to express a desired protein, for example, to express the desired protein selectively in tumor cells after infection of the tumor cells. In some embodiments, the delivery systems and methods are “personalized”, meaning the cancer stem cells are harvested from the subject receiving the treatment. All treatments taught herein can be personalized, whether it be administration of the programmed cancer stem cells alone as a cancer vaccine to deliver cancer antigens; administration of the programmed cancer stem cells in which the cancer stem cells have been engineered to deliver an oncolytic virus with the cancer antigens; or, administration of the programmed cancer stem cells with an oncolytic virus expressing a desired protein to deliver the cancer specific antigens, the oncolytic virus, and the desired protein.

As such, in some embodiments, the cancer stem cells are engineered to provide a “triple-killer” cancer treatment that includes administering live, cancer stem cells that are re-programmed to die in a desired amount of time after administration to a subject and release cancer antigens as a vaccine; administering an oncolytic virus through the host cancer stem cells that were further engineered with the oncolytic virus; and, administering a desired protein, perhaps an immunomodulatory cytokine, by engineering the oncolytic virus to express the desired protein. The triple-killer cancer treatment does 3 things: (1) kills the cancer stem cell after administration in a subject to release a cancer antigen cocktail that creates cancer antibodies in the subject; (2) kills additional cancer tumor cells in the subject through release of the oncolytic virus itself; and (3) kills additional cancer tumor cells in the subject with the activity from the desired protein. And, interestingly, the delivery of the desired protein, such as an immunomodulatory cytokine, can further provide an additional immunological memory response. Moreover, since the cancer stem cells can be cancer-specific, each of the systems and methods taught herein can be “cancer-specific” to treat, at least inhibit, or perhaps even prevent a specific type of cancer that develops from the cancer stem cells. Those of skill will understand and appreciate, however, that each of the systems and methods taught herein can be effective at treating a variety of cancers that extend well beyond the specific type of cancer that develops from the cancer stem cells.

Moreover, in the delivery systems and methods provided herein, the oncolytic virus is amplified in the cancer stem cell through replication. This is an added benefit because the built-in amplification mechanism means that the patient does not have to receive a much larger initial dose of an oncolytic virus, or desired protein, which is the case when systemically administering the oncolytic virus and/or desired protein alone, using state-of-the-art techniques. One of skill will appreciate that the larger dose is needed to offset the elimination of the oncolytic virus and/or desired protein by the subject's immune system before the treatment has a chance to reach the target cancer cells. As such, the larger initial dose is known to create an undesired toxicity which can be uncomfortable, harmful, or even fatal to the subject. While not intending to be bound by any theory or mechanism of action, the use of the programmed cancer stem cells as a delivery vehicle protects the subject from the oncolytic virus and/or desired protein until, and the oncolytic virus and/or desired protein from the subject, until the cancer stem cells die and release the agents for the treatment. The state-of-the-art is improved by the systems and methods taught herein. The subject treated is exposed to less toxicity, the cancer stem cells provide a machine to amplify the oncolytic virus after administration, the half-life of the oncolytic virus and/or protein is increased by the cancer stem cells shielding them from the subjects immune response, and less of the toxic agents need to be administered to the subject through cancer stem cells as compared to the state-of-the-art administrations of the agents alone through I.V. or I.M. routes of administration.

One of ordinary skill will appreciate that all of the systems and methods taught herein can be used in treating, inhibiting the onset of, or perhaps even preventing, any solid cancers or liquid cancers in a subject.

In some embodiments, the teachings are directed to a general method of creating a delivery system for cancer stem cell antigens and an oncolytic virus. The method can include obtaining live cancer stem cells, programming the live cancer stem cells to die in a desired amount of time, loading the live cancer stem cells with an oncolytic virus, administering the live cancer stem cells in an environment in which they will die and release antigens from the dead cancer stem cells and release the oncolytic virus.

In some embodiments, the teachings are directed to a general method of creating a delivery system for cancer stem cell antigens and an oncolytic virus that expresses a desired protein. The method can include obtaining live cancer stem cells, programming the live cancer stem cells to die in a desired amount of time, loading the live cancer stem cells with an oncolytic virus that expresses a desired protein, administering the live cancer stem cells in an environment in which they will die and release antigens from the dead cancer stem cells, release the oncolytic virus that expresses the desired protein.

In some embodiments, the teachings are directed to a general method of creating a delivery system for cancer stem cell antigens and an oncolytic virus, the delivery system administered to a subject The method can include obtaining live cancer stem cells, programming the live cancer stem cells to die in a desired amount of time, loading the live cancer stem cells with an oncolytic virus, administering the live cancer stem cells in an environment in the subject in which they will die and release antigens from the dead cancer stem cells and release the oncolytic virus.

In some embodiments, the teachings are directed to a general method of creating a delivery system for cancer stem cell antigens and an oncolytic virus that expresses a desired protein, the delivery system administered to a subject. The method can include obtaining live cancer stem cells, programming the live cancer stem cells to die in a desired amount of time, loading the live cancer stem cells with an oncolytic virus that expresses a desired protein, administering the live cancer stem cells in an environment in the subject in which they will die and release antigens from the dead cancer stem cells, release the oncolytic virus that expresses the desired protein.

### Administering the Programmed Cancer Stem Cells as a Vaccine.

We have shown that the cancer stem cells can be used as an anticancer vaccine, using glioblastoma stem cells isolated from an orthotopic glioblastoma brain tumor. We found that the anticancer vaccine provided (i) 100% protection against the development of glioblastoma, (ii) long-term survival, and (iii) no obvious toxicity.

The systems and methods of treatment taught herein can be used with all cancers, whether solid cancers or liquid cancers. The cancer stem cells can be cancer for a solid cancer or a liquid cancer, and they can be taken directly from the subject to be treated, or from a donor. In some embodiments, the cancer stem cells are taken directly from the subject treated to personalize the treatment. As an anticancer vaccine, the cancer stem cells are programmed to die in a desired time after administration, administered live, die in the desired time frame to release a cocktail of antigens into the subject to incite an immune response.

In some embodiments, a cancer vaccine for treating, or at least inhibiting the onset of, a solid cancer in a subject is provided. In some embodiments, the vaccine can include a carrier. In some embodiments, reprogrammed cancer stem cells created using a process including collecting live cancer stem cells from the solid cancer tissue from a donor; and, reprogramming the live cancer stem cells to die in a programmed time-frame; the reprogramming including treating the live cancer stem cells with an effective amount of an apoptosis inducing agent. In these embodiments, the reprogrammed cancer stem cells die after being administered to the recipient; and, the death of the reprogrammed cancer stem cells results in boosting cancer immunity in the recipient through a release of antigens into the recipient. The boosting can include releasing cancer stem cell antigens from the reprogrammed cancer stem cells; and, stimulating the subject's immune system; wherein, the releasing and stimulating resulting in boosting the cancer immunity against the solid cancer.

In some embodiments, the vaccine is a cancer vaccine for a liquid cancer a subject. In some embodiments, the vaccine can include a carrier. In some embodiments, reprogrammed cancer stem cells created using a process including collecting live cancer stem cells from the liquid cancer tissue from a donor; and, reprogramming the live cancer stem cells to die in a programmed time-frame; the reprogramming including treating the live cancer stem cells with an effective amount of an apoptosis inducing agent. In these embodiments, the reprogrammed cancer stem cells die after being administered to the recipient; and, the death of the reprogrammed cancer stem cells results in boosting cancer immunity in the recipient through a release of antigens into the recipient. The boosting can include releasing cancer stem cell antigens from the reprogrammed cancer stem cells; and, stimulating the subject's immune system; wherein, the releasing and stimulating resulting in boosting the cancer immunity against the liquid cancer.

One of skill will appreciate that the cancer stem cells can include any stem cell from any cancer. The cancer stem cells can be collected from a solid cancer or a liquid cancer. In some embodiments, the cancer stem cell is collected from a solid tumor selected from the group consisting of a carcinoma, a sarcoma, a germ cell tumor, and a blastoma. In some embodiments, the cancer stem cells are collected from a tissue donor.

In some embodiments, the cancer is a solid cancer, and the cancer stem cell is collected from a bone and muscle sarcoma selected from the group consisting of chondrosarcoma, Ewing's sarcoma, malignant fibrous histiocytoma of bone/osteosarcoma, osteosarcoma, rhabdomyosarcoma, leiomyosarcoma, myxosarcoma, and fibrocartilaginous mesenchymoma of bone.

In some embodiments, the cancer is a solid cancer, and the cancer is a solid cancer, and the cancer stem cell is collected from a brain and nervous system cancer selected from the group consisting of astrocytoma, brainstem glioma, pilocytic astrocytoma, ependymoma, primitive neuroectodermal tumor, cerebellar astrocytoma, cerebral astrocytoma, glioblastoma, glioma, medulloblastoma, neuroblastoma, oligodendroglioma, pineal astrocytoma, pituitary adenoma, and visual pathway and hypothalamic glioma.

In some embodiments, the cancer is a solid cancer, and the cancer stem cell is collected from a breast cancer selected from the group consisting of breast cancer, inflammatory breast cancer, invasive lobular carcinoma, tubular carcinoma, invasive cribriform carcinoma of the breast (also termed invasive cribriform carcinoma, medullary carcinoma, male breast cancer, phyllodes tumor, mammary secretory carcinoma, and papillary carcinomas of the breast.

In some embodiments, the cancer is a solid cancer, and the cancer stem cell is collected from a cancer of the endocrine system selected from the group consisting of adrenocortical carcinoma, islet cell carcinoma (endocrine pancreas), multiple endocrine neoplasia syndrome, parathyroid cancer, pheochromocytoma, thyroid cancer, and merkel cell carcinoma.

In some embodiments, the cancer is a solid cancer, and the cancer stem cell is collected from a cancer of the eye selected from the group consisting of uveal melanoma, retinoblastoma, and optic nerve glioma.

In some embodiments, the cancer is a solid cancer, and the cancer stem cell is collected from a gastrointestinal cancer selected from the group consisting of anal cancer, appendix cancer, cholangiocarcinoma, carcinoid tumor gastrointestinal, colon cancer, extrahepatic bile duct cancer, gallbladder cancer, gastric (stomach) cancer, gastrointestinal carcinoid tumor, gastrointestinal stromal tumor (gist), hepatocellular cancer, pancreatic cancer islet cell, rectal cancer, and small intestine cancer.

In some embodiments, the cancer is a solid cancer, and the cancer stem cell is collected from a genitourinary and gynecologic cancer selected from the group consisting of bladder cancer, cervical cancer, endometrial cancer, extragonadal germ cell tumor, ovarian cancer, ovarian epithelial cancer (surface epithelial-stromal tumor), ovarian germ cell tumor, penile cancer, kidney cancer, renal cell carcinoma, renal pelvis and ureter, transitional cell cancer, prostate cancer, testicular cancer, gestational trophoblastic tumor, ureter and renal pelvis, transitional cell cancer, urethral cancer, uterine sarcoma, vaginal cancer, vulvar cancer, and wilms tumor (nephroblastoma).

In some embodiments, the cancer is a solid cancer, and the cancer stem cell is collected from a head and neck cancer selected from the group consisting of esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer.

In some embodiments, the cancer is a solid cancer, and the cancer stem cell is collected from a skin cancer selected from the group consisting of basal cell carcinoma, squamous cell carcinoma, squamous cell skin cancer, skin adnexal tumors (e.g. sebaceous carcinoma), melanoma, merkel cell carcinoma, keratoacanthoma, sarcomas of primary cutaneous origin (e.g. dermatofibrosarcoma protuberans), and lymphomas of primary cutaneous origin (e.g. mycosis fungoides).

In some embodiments, the cancer is a solid cancer, and the cancer stem cell is collected from a thoracic and respiratory cancer selected from the group consisting of adenocarcinoma of the lung, bronchial adenomas/carcinoids, small cell lung cancer, mesothelioma, non-small cell lung cancer, non-small cell lung carcinoma, pleuropulmonary blastoma, laryngeal cancer, thymoma and thymic carcinoma, and squamous-cell carcinoma of the lung.

In some embodiments, the cancer is a solid cancer, and the cancer stem cell is collected from a kaposi sarcoma, an epithelioid hemangioendothelioma (ehe), a desmoplastic small round cell tumor, or a liposarcoma.

In some embodiments, the cancer is a liquid cancer, and the cancer stem cell is collected from a leukemia, a lymphoma, or a multiple myeloma.

In some embodiments, the cancer is a liquid cancer, and the cancer stem cell is collected from a hematopoietic cancer selected from the group consisting of acute biphenotypic leukemia, acute eosinophilic leukemia, acute lymphoblastic leukemia, acute myeloid leukemia, acute myeloid dendritic cell leukemia, aids-related lymphoma, anaplastic large cell lymphoma, angioimmunoblastic t-cell lymphoma, b-cell prolymphocytic leukemia, Burkitt's lymphoma, chronic lymphocytic leukemia, chronic myelogenous leukemia, cutaneous t-cell lymphoma, diffuse large b-cell lymphoma, follicular lymphoma, hairy cell leukemia, hepatosplenic t-cell lymphoma, Hodgkin's lymphoma, intravascular large b-cell lymphoma, large granular lymphocytic leukemia, lymphoplasmacytic lymphoma, lymphomatoid granulomatosis, mantle cell lymphoma, marginal zone b-cell lymphoma, mast cell leukemia, mediastinal large b cell lymphoma, myelodysplastic syndromes, mucosa-associated lymphoid tissue lymphoma, mycosis fungoides, nodal marginal zone b cell lymphoma, non-Hodgkin lymphoma, precursor b lymphoblastic leukemia, primary central nervous system lymphoma, primary cutaneous follicular lymphoma, primary cutaneous immunocytoma, primary effusion lymphoma, plasmablastic lymphoma, Sézary syndrome, splenic marginal zone lymphoma, and t-cell prolymphocytic leukemia.

In some embodiments, the cancer is a liquid cancer, and the cancer stem cell is collected from an acute myeloid leukemia, a chronic myeloid leukemia, an acute lymphocytic leukemia, or a chronic lymphocytic leukemia.

In some embodiments, the cancer is a liquid cancer, and the cancer stem cell is collected from a Hodgkin's lymphoma, or a non-Hodgkin's lymphoma.

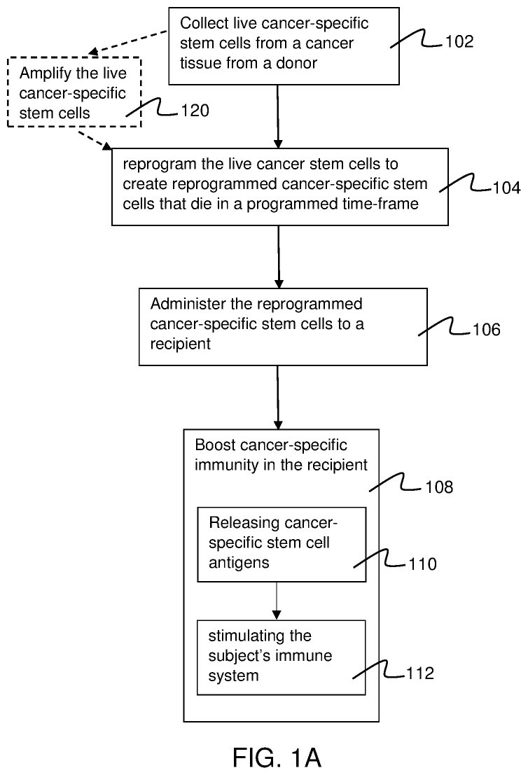

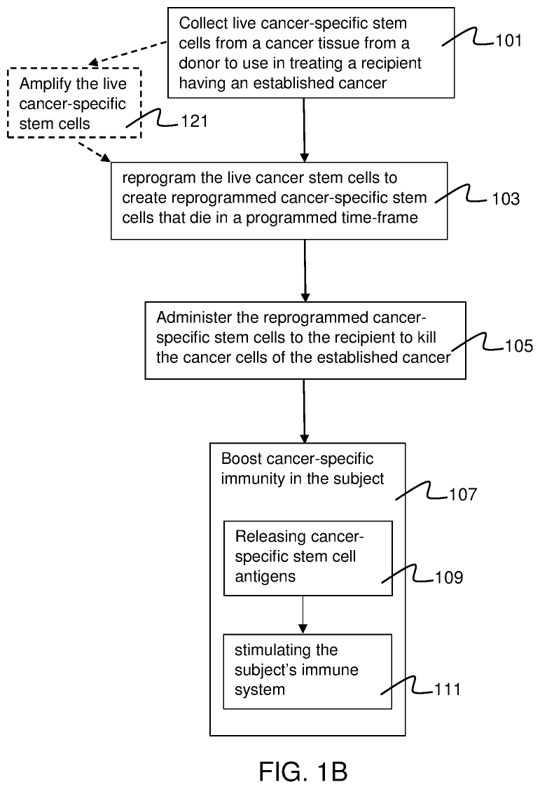

FIGS. 1A and 1B are flowcharts showing a general method of using apoptotic stem cells as a cancer vaccine, according to some embodiments. FIG. 1A shows steps in a method used to at least inhibit, or prevent, the development of a cancer in a subject. The method includes collecting 102 live, cancer stem cells from a cancer tissue from a donor, and reprogramming 104 the live cancer stem cells to create reprogrammed cancer stem cells that die in a programmed time-frame for use as the active components in an anti-cancer vaccine.

The cancer stem cells are host cells that can be transfected or transformed with the expression or cloning vectors described herein. The host cells are the cancer stem cells. In some embodiments, the cells can be cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra, each of which are incorporated by reference. The cancer stem cells can be cultured in vitro or genetically engineered using techniques known in the art. In some embodiments, the cancer stem cells can be harvested from normal subjects or affected subjects, including healthy humans, cancer patients. In some embodiments, the cancer stem cells can be harvested from the subject being treated. And, in some embodiments the cancer stem cells can be stored in, and obtained from a private laboratory deposit, public culture collections such as the American Type Culture Collection. It is contemplated that, in some embodiments, the cancer stem cells can be prepared by, and obtained from, commercial suppliers for use with the methods taught herein.

The harvested tissue needs to be grown to obtain a sufficient amount of cellular material. In some embodiments, tissue harvested from a tumor can be enzymatically digested by type IV collagenase, followed by collection of disaggregated cells. The disaggregated cells can then be grown in vitro in growth media with 10% fetal bovine serum on an extracellular matrix substrate, such as collagen or fibronectin, to promote attachment. Adherent cells can then be passaged until immortal cancer cells outgrow the non-cancerous fibroblast cells.

The next step is the administering 106 of the reprogrammed cancer stem cells to a recipient, the dying of the cancer stem cells occurring after administering the reprogrammed cancer stem cells to the recipient to treat, or at least inhibit a development of, the cancer in the recipient. The administering 106 leads to the next step of a boosting 108 of a cancer immunity in the recipient, the boosting 108 including releasing 110 cancer stem cell antigens, and stimulating 112 the recipient's immune system, wherein, the releasing and stimulating results in boosting the cancer immunity against the solid cancer. FIG. 1B, on the other hand, shows steps in a method used as a treatment for an established cancer to at least kill cancer stem cells and, preferably, eradicate the cancer from a subject. The method includes collecting 101 live, cancer stem cells from a cancer tissue from a donor to use in treating a recipient having an established cancer, and reprogramming 103 the live cancer stem cells to create reprogrammed cancer stem cells that die in a programmed time-frame for use as the active component in an anti-cancer vaccine. The next step is the administering 105 of the reprogrammed cancer stem cells to a subject to kill cancer cells of the established cancer in the subject/recipient and, preferably, eradicate the cancer. The administering 105 leads to the next step of a boosting 107 of a cancer immunity in the subject, the boosting 107 including releasing 109 cancer stem cell antigens, and stimulating 111 the recipient's immune system against the cancer. One of skill will appreciate that the additional step of amplifying 120,121 the live cancer stem cells can be done to create a sufficient number of stem cells for the creation of a desired quantity of vaccine. It should also be appreciated that, in some embodiments, the donor is the subject, such that the donor is the recipient of the vaccine. Any method of amplifying the cells known to one of skill can be used. In vitro amplification, for example, is a well-known procedure that can be easily done by the skilled artisan.

One of skill will appreciate that the collecting 101,102 of the live, cancer stem cells includes cell separation. Cell separation isolates the cancer stem cells from the cancer tissue. Any method of cell separation known to one of skill can be used. Examples of such techniques known in the art include, but are not limited to, adherence, density and antibody binding. Examples of separation types used in the art include fluorescence-activated cell sorting (FACS), magnet-activated cell sorting (MACS), pre-plating, conditioned expansion media, density gradient centrifugation, field flow fractionation (FFF), and dielectrophoresis (DEP). See, for example, Zhu, B. et al. Curr Opin Chem Eng.; 2(1): 3-7 (2013), incorporated herein by reference in its entirety. Newer techniques include microfluidics that use cellular properties such as elasticity in response to acoustic waves, and membrane polarization in a non-uniform electric field.

The antibody binding type of separation uses the expression of cell surface markers to isolate the stem cells. In some embodiments, the cell surface markers used to separate the cells can be selected from the group consisting of CD44, CD24, CD29, CD90, CD133, CD117, and CD166, as well as epithelial-specific antigen (ESA), aldehyde dehydrogenase1 (ALDH1), and combinations thereof. These markers can be used to isolate and enrich cancer stem cells from different tumors, because the expression of cancer stem cell surface markers is tissue type-specific, and even tumor subtype-specific. For example, CD44+CD24−/low lineage and ALDH+ can be used to isolate breast cancer stem cells; CD133+ can be used to isolate colon, brain and lung cancer stem cells; CD34+CD8− can be used to isolate leukemia cancer stem cells; CD44+ can be used to isolate head and neck cancer stem cells; CD90+ can be used to isolate liver cancer stem cells; and CD44+/CD24+/ESA+ can be used to isolate pancreas cancer stem cells. See at least Int J Biochem Cell Biol. 44(12): 2144-2151 (December 2012) https://www.ncbi.nlm.nih.gov/pmc/articles/PMC3496019/ (downloaded Aug. 29, 2022), incorporated herein by reference in it's entirety. Other marker for cancer stem cells include SOX2, OCT4, Nanog and Nestin, each of which can be found in cancers that include at least nasopharyngeal carcinoma. See at least PLoS One. 2013; 8(2): e56324; doi: 10.1371/journal.pone.0056324 (Feb. 12, 2013) (downloaded Sep. 2, 2022), incorporated herein by reference in it's entirety.

The reprogramming 103 of the live cancer stem cells to die in a desired time-frame can be done in several ways known to those of ordinary skill in the art. Any method of inducing apoptosis in the cancer stem cell can be used, in some embodiments. An apoptosis inducing agent can be any agent used to treat cancer herein, including a cytotoxic agent such as radiation therapy or chemotherapy, for example. Cancer stem cells that are pre-treated with radiation to die in the desired time-frame can be referred to as “radiation-primed” cancer stem cells, in some embodiments.

Radiation therapy can use ionizing radiation, for example, such as X-rays, gamma rays, electron beams, protons, UVA, UVB, or a combination thereof, to induce apoptosis in a cell. As such, the radiation therapy can also be used to program the cancer stem cells to die in a desired time-frame. In some embodiments, the reprogramming of the cancer stem cells can use radiation at a total dose of 25 Gy, 30 Gy, 35 Gy, 40 Gy, 45 Gy, 50 Gy, 55 Gy, 60 Gy, 65 Gy, 70 Gy, 75 Gy, 80 Gy, 85 Gy, 90 Gy, 95 Gy, 100 Gy, 105 Gy, 110 Gy, 115 Gy, 120 Gy, 125 Gy, 130 Gy, 135 Gy, 140 Gy, or any amount or range therein in increments of 1 Gy. In some embodiments, the total radiation dose can range from 25 Gy to 140 Gy, from 25 Gy to 125 Gy, from 30 Gy to 100 Gy, from 30 Gy to 75 Gy, from 30 Gy to 50 Gy, from 35 Gy to 45 Gy, or any amount or range therein in increments of 1Gy. See, for example, Venkatesulu, B. P., et al. (ultra high dose rate (35 Gy/sec) radiation does not spare the normal tissue in cardiac and splenic models of lymphopenia and gastrointestinal syndrome). Scientific Reports 9(1):17180 (2019) (uses ultra high does rate electron irradiation, 4.5 MeV at 40 Gy/s), incorporated herein by reference in its entirety.

It should be appreciated that the amount of time it takes for the cancer stem cells to die can be referred to as a “time to death”, “a time to cell death”, “programmed time”, “programmed time frame”, “desired time”, “desired time frame”, and the like. The time to death of the cell depends on the treatment used to program the cell. If radiation is used, for example, the time to death of the cancer stem cells depends on the amount of exposure, meaning the total dose of radiation applied to the stem cells. The skilled artisan can select an apoptosis agent to use in the programming, and amount administered, to program the death of the live cancer stem cells. In some embodiments, the live cancer stem cells die in 1-21 days, 2-18 days, 12-14 days, in 10-12 days, 10-14 days, 1-7 days, 1-4 days, 2-10 days, 7-14 days, or any amount or range therein in increments of 1 day. In some embodiments, the stem cells die in 1 day, 2 days, 3 days, 4 days, 5 days, 6 days, 7 days, 8 days, 9 days, 10 days, 11 days, 12 days, 13 days, 14 days, 15 days, 16 days, 17 day, 18 days, 19 days, 20 days, 21 days, or any amount or range therein in increments of 1 hour.

The agent used to program the time to death of the cancer stem cells can be called a programming agent, in some embodiments. In some embodiments, the programming agent is radiation, and where the total irradiation exposure is about 35 Gy, the programmed cell death is about 10-12 days. It may be desirable for cell death to occur sooner, and a higher dose can be used. In some embodiments, a higher dose ranging from 35 Gy to 100 Gy, or any range therein, can be used to speed up apoptosis. In some embodiments, a lower dose ranging from 25 Gy to 35 Gy may be used for cell death to occur more slowly. The irradiation time depends on the dose rate of the irradiation device. For example, the desired total dose of radiation can be obtained in an amount of time that varies depending on the rate of radiation provided by the radiation device. For example, if the dose rate is 0.740 Gy/min, cancer stem cells would be exposed to radiation for ˜47 minutes to obtain a total exposure of 35 Gy.

Chemotherapeutic agents can be used to program the time to death of the cancer stem cells. In some embodiments, the programming agent is a chemotherapeutic agent taught herein. One of skill can readily select the type and amount of chemotherapeutic agent to use in the programming using standing in vitro methods that will test the toxicity of the agent to the cancer stem cells over time. The amount of therapeutic agent to use in the programming can be an amount taught herein for administration to a subject as a chemotherapeutic treatment.

An oncolytic virus can be used to program the time to death of the cancer stem cells. In some embodiments, the oncolytic virus can be an oncolytic virus taught herein. In some embodiments, the loading of the cancer stem cell with an oncolytic virus taught herein can program the cancer stem cells to die in the desired time frame. In some embodiments, the oncolytic virus is engineered to express a desired protein, and the protein expressed can be selected to not only treat the subject receiving the administration of the programmed cancer stem cells, but also to set the time to death of the cancer stem cells after they're administered to the subject.

It should be appreciated that any of the agents taught herein to treat a cancer, or combinations of those agents, can also be used for reprogramming the live cancer stem cells to die in a programmed time-frame after administration to a subject. In some embodiments, the agent is a cytotoxic agent, and the ordinary skilled artisan can select a cytotoxic agent and use a screening assay to readily determine the dose of the cytotoxic agent to administer for the cancer stem cells to die in a desired, programmed amount of time after administration to a subject. In fact, any agent taught herein can be screened to assess a relationship between amount of agent administered to the cancer stem cells and time to death to reprogram the cancer stem cells and use them as a vaccine as desired.

The administering 103,104 of the dying cancer stem cells to the subject can be done using any known method of administration. In some embodiments, the cancer stem cells can be administered intravenously to deliver the vaccine systemically. In some embodiments, the cancer stem cells can be administered locally, at the site of a target tumor. In some embodiments, the cancer stem cells can be administered intraperitoneally (e.g., administer locally for gastrointestinal cancer). And, in some embodiments, the cancer stem cells can be administered intravesically (e.g., administer locally for bladder cancer).

As noted, in some embodiments, the isolated live cancer stem cells from a donor are amplified to create an amount of stem cells desired to create an amount of vaccine desired. In some embodiments, the amplification of the isolated stem cells from a donor may be done to create a single dose of vaccine for a single recipient, or to create a plurality of doses of a vaccine for a single recipient. In some embodiments, the amplification of the isolated stem cells from a donor may be done to create several doses of a vaccine for several recipients.

In any event, the boosting 107 of the cancer immunity in the subject includes releasing 109 cancer stem cell antigens in the subject from the cancer stem cells, and stimulating 111 the subject's immune system.

Combining the Methods Taught Herein with Other Therapies as a Combination Administration

It should be appreciated that any of the therapies taught herein can be used together in some embodiments, regardless of whether the therapy is the administration of the cancer stem cells alone, the cancer stem cells loaded with an oncolytic virus, or the cancer stem cells loaded with an onocolytic virus that expresses a desired protein. As such, the cancer stem cells taught herein can be used in combination with a secondary therapeutic agent that can be selected from a number of different types of agents having a number of different types of activities and mechanisms of action.

The rationale for combination therapies is to combine different mechanisms of action to decrease the likelihood that resistant cancer cells will develop. When drugs having different effects are combined, each drug can be used at its optimal dose, in some embodiments. Live cancer stem cells that are programmed to die in a desired time frame can be administered live to a subject, die in the subject in the programmed, desired time frame, and release antigens in the subject that lead to a cancer immune response.

As such, the programmed cancer stem cells can be administered in combination with other therapies selected from the group consisting of immunotherapy, chemotherapy, radiation therapy, targeted therapies, oncolytic virus therapies, and any combinations thereof. In some embodiments, targeted therapies can include angiogenesis therapy, hormonal therapy, thermotherapy, or a combination thereof. For example, the oncolytic virus can express an immunomodulating protein, a fusogenic protein, or a combination of an immunomodulating protein and a fusogenic protein. One of skill will appreciate that the agents within each type of such a secondary therapy can also be combined. For example, a plurality of different fusogenic proteins can be used in combination with the programmed cancer stem cells in some embodiments. Likewise, a plurality of different immunomodulating proteins can be used in combination with the programmed cancer stem cells in some embodiments.

Regardless of the secondary therapy used in conjunction with an oncolytic virus delivered by the cancer stem cells, administration of the cancer stem cells and the secondary therapy can be in parallel or in series. In some embodiments, the cancer stem cells can be administered before the administration of the secondary therapy. In some embodiments, the cancer stem cells can be administered with the administration of the secondary therapy. In some embodiments, the cancer stem cells can be administered after the administration of the secondary therapy.

The methods of treating cancer taught herein may comprise multiple administrations of the cancer stem cells, cancer stem cells loaded with oncolytic virus, and/or cancer stem cells loaded with oncolytic virus engineered to express a desired protein. Likewise, multiple administrations of the second therapy may also be needed or desired. A person of ordinary skill can determine suitable courses of administration of the oncolytic virus and the immune co-inhibitory pathway and/or an agonist of the immune co-stimulatory pathway.

In some embodiments, doses can be administered between 2 days to 12 weeks apart, 3-days to 3 weeks apart, or any range or duration therein in increments of 1 day. In some embodiments, doses can be repeated for up to 1 year, 2 years, 3 years, 4 years, 5 years, or more than 5 years. In some embodiments, doses can be repeated for a range of one month to two years, two weeks to 3 years, 1 week to 4 years, 2 weeks to 1 year, or any range or duration therein in increments of 1 day. One of skill will appreciate that the course of treatment selected will depend on the how the subject responds to the treatment, how the cancer tissue responds to the treatment, or a combination thereof. In some embodiments, the course of treatment can depend on the speed of response of the tumor to the administration of the cancer stem cells loaded with the oncolytic virus and, optionally, any combination therapy which may also be being given.

In some embodiments, the secondary therapy can include chemotherapy, targeted therapy, immunotherapy (including immune co-inhibitory pathway blockade or immune co-stimulatory pathway activation), radiation therapy, or any combination thereof. In some embodiments, two types of a second therapeutic agent can be combined to administer in combination with the cancer stem cells. For example, the delivery of a first oncolytic virus can be combined with the delivery of a second oncolytic virus. As such, combining the programmed cancer stem cell vaccine with one or more other therapies can effectively treat cancer, including at least inhibiting the onset of cancer, and sometimes even prevent cancer in a subject.

### Immunotherapy

The cancer stem cells taught herein can be used in combination with active or passive immunotherapies and immunomodulators and, in some embodiments are administered as a secondary therapeutic agent.

In some embodiments, the second therapeutic agent can include an immunogen (including a recombinant or naturally occurring antigen, including such an antigen or combination of antigens delivered as DNA or RNA in which it/they are encoded), to further stimulate an immune response, such as a cellular or humoral immune response, to tumor cells, particularly tumor neoantigens.

In some embodiments, the second therapeutic agent can include agents that target a specific genetic mutation which occurs in tumors, agents that induce immune responses to specific tumor antigens or combinations of tumor antigens, including agents intended to activate the STING/cGAS pathway, TLR or other innate immune response and/or inflammatory pathway, including intra-tumoral agents.

In some embodiments, the second therapeutic agent can include an active CAR-T cell therapy, including harvesting T cells, genetically altering the T cells to add a chimeric antigen receptor (CAR) that specifically recognizes cancer cells, and infusing the resulting CAR-T cells into patients to attack their tumors. In some embodiments, an immunotherapeutic agent can include bi-specific antibodies, cell based-therapies based on dendritic cells, NK cells or T cells expressing engineered T cell receptors.

In some embodiments, the second therapeutic agent is selected to inhibits an immune checkpoint pathway or stimulate an immune potentiating pathway or an agent which inhibits the activity of regulatory T cells (Tregs). In some embodiments, the immunotherapy is a passive checkpoint inhibitor. In some embodiments, the cancer stem cells can be administered with an immune checkpoint blockade such as anti-PD-1, or anti-CTLA4 that will further activate T cells and efficacy of the cancer stem cells, as the cancer stem cells appear to be CD4 dependent. In some embodiments, immune checkpoint antagonists include antibodies, single chain antibodies and RNA1/siRNA/microRNA/antisense RNA knockdown approaches. In some embodiments, the second therapeutic agent can be selected from agents designed to block immune checkpoints or stimulate immune potentiating pathways, including but not limited to monoclonal antibodies, such as a CTLA-4 inhibitor, a PD-1 inhibitor, a PD-L1 inhibitor, a LAG-3 inhibitor, a TIM-3 inhibitor, a VISTA inhibitor, a CSF1R inhibitor, an IDO inhibitor, a CEACAM1 inhibitor, a GITR agonist, a 4-1-BB agonist, a KIR inhibitor, a SLAMF7 inhibitor, an OX40 agonist, a CD40 agonist, an ICOS agonist or a CD47 inhibitor. In a preferred embodiment, the therapeutic agent is a CTLA-4 inhibitor such as an anti-CTLA-4 antibody, a PD1 inhibitor, such as an anti-PD-1 antibody or a PD-L1 inhibitor such as an anti-PD-L1 antibody. Such inhibitors, agonists and antibodies can be generated and tested by standard methods known in the art. In some embodiments, the second therapeutic agent may be an inhibitor of the idoleamine 2,3-dioxygenase (IDO) pathway. Examples of IDO inhibitors include epacadostat (INCB024360), 1-methyl-tryptophan, indoximod (1-methyl-D-tryptophan), GDC-0919 or F001287. IDO inhibitors can be effective because the mechanism of action of IDO in suppressing anti-tumor immune responses may also suppress immune responses generated following oncolytic virus therapy. IDO expression is induced by toll like receptor (TLR) activation and interferon-γ both of which may result from oncolytic virus infection. In some embodiments, an oncolytic virus can be administered with an inhibitor of the IDO pathway. And, in some embodiments, the oncolytic virus can be administered with an inhibitor of the IDO pathway and an antagonist of an immune co-inhibitory pathway and/or an agonist of an immune co-stimulatory pathway, including those targeting CTLA-4, PD-1 and/or PD-L1.

In some embodiments, the second therapeutic agent can include cytokine therapy, where the cytokine therapy is an interleukin therapy including, but not limited to, interleukin-2. In some embodiments, the cytokine therapy is an interferon therapy including, but not limited to, interferon-α. Interleukin-2 and interferon-α regulate and coordinate the immune system, enhance anti-tumor activity and provide passive cancer treatments. Interferon-α is used in the treatment of hairy-cell leukaemia, AIDS-related Kaposi's sarcoma, follicular lymphoma, chronic myeloid leukemia and malignant melanoma. Interleukin-2 is used in the treatment of malignant melanoma and renal cell carcinoma.

In some embodiments, the second therapeutic agent can include antibody therapy. Any antibody known to one of skill can be used with the methods taught herein. Antibody therapy provides additional bioactive agents that may be useful when administered in combination with the methods taught herein. AVASTATIN, for example, is a human monoclonal antibody to VEGF, has provided beneficial results in colorectal cancer, increasing survival time by more than 30% when used in combination with the standard Saltz regime of irinotecan, 5-fluorouracil, and leucovorin. Approved checkpoint inhibitors include antibodies such as ipilimumab, nivolumab, and pembrolizumab. One of skill will appreciate that several monoclonal antibodies would be useful, the following providing further examples:

TABLE 1

| mAb name | Trade name | Cancer treated: |
|---|---|---|
| rituximab | RITUXAN | non-Hodgkin lymphoma |
| trastuzumab | HERCEPTIN | breast cancer |
| gemtuzumab | MYLOTARG | acute myelogenous leukemia |
| ozogamicin* | | (AML) |
| alemtuzumab | CAMPATH | chronic lymphocytic leukemia |
| | | (CLL) |
| ibritumomab tiuxetan* | ZEVALIN | non-Hodgkin lymphoma |
| tositumomab* | BEXXAR | non-Hodgkin lymphoma |
| cetuximab | ERBITUX | colorectal cancer; head & |
| | | neck cancers |
| bevacizumab | AVASTIN | colorectal cancer; non-small |
| | | cell lung cancer; breast cancer; |
| | | glioblastoma; kidney cancer |
| panitumumab | VECTIBIX | colorectal cancer |
| ofatumumab | ARZERRA | chronic lymphocytic leukemia |
| | | (CLL) |
| atezolizumab | TECENTRIQ | bladder cancer |
| avelumab | BAVENCIO | metastatic Merkel cell |
| | | carcinoma |
| ipilimumab | YERVOY | metastatic melanoma |
| elotuzumab | EMPLICITI | multiple myeloma |
| ofatumumab | ARZERRA | refractory CLL |
| nivolumab | OPDIVO | unresectable or metastatic |
| | | melanoma, squamous non-small |
| | | cell lung cancer, Renal cell |
| | | carcinoma, colorectal cancer, |
| | | hepatocellular carcinoma, |
| | | classical Hodgkin lymphoma |
| pembrolizumab | KEYTRUDA | unresectable or metastatic |
| | | melanoma, squamous non-small |
| | | cell lung cancer (NSCLC), |
| | | Hodgkin's lymphoma, Merkel- |
| | | cell carcinoma (MCC), primary |
| | | mediastinal B-cell lymphoma |
| | | (PMBCL), stomach cancer, |
| | | cervical cancer |
| durvalumab | IMFINZI | bladder cancer non-small cell |
| | | lung cancer |

*refers to a conjugated monoclonal antibody

### Chemotherapy

The cancer stem cells taught herein can be used in combination with a chemotherapeutic agent administered as a secondary therapeutic agent. Any chemotherapy drug known to one of skill can be used with the methods taught herein and, in some embodiments are administered as a secondary therapeutic agent. Antiproliferatives include, for example, actinomycin D, actinomycin IV, actinomycin 11, actinomycin X1, actinomycin C1, and dactinomycin (Cosmegen®, Merck & Co., Inc.). Antineoplastics or antimitotics include, for example, paclitaxel (TAXOL, Bristol-Myers Squibb Co.), docetaxel (TAXOTERE, Aventis S.A.), methotrexate, irinotecan, SN-38, azathioprine, vincristine, vinblastine, fluorouracil, doxorubicin hydrochloride (ADRIAMYCIN, Pfizer, Inc.) and mitomycin (MUTAMYCIN, Bristol-Myers Squibb Co.), and any prodrugs, codrugs, metabolites, analogs, homologues, congeners, derivatives, salts and combinations thereof. Cytostatic or antiproliferative agents include, for example, angiopeptin, angiotensin converting enzyme inhibitors such as captopril (CAPOTEN and CAPOZIDE, Bristol-Myers Squibb Co.), cilazapril or lisinopril (PRINVIL and PRINZIDE, Merck & Co., Inc.); calcium channel blockers such as nifedipine; colchicines; fibroblast growth factor (FGF) antagonists, fish oil (omega 3-fatty acid); histamine antagonists; lovastatin (MEVACOR, Merck & Co., Inc.); monoclonal antibodies including, but not limited to, antibodies specific for Platelet-Derived Growth Factor (PDGF) receptors; nitroprusside; phosphodiesterase inhibitors; prostaglandin inhibitors; suramin; serotonin blockers; steroids; thioprotease inhibitors; PDGF antagonists including, but not limited to, triazolopyrimidine; and nitric oxide, and any prodrugs, codrugs, metabolites, analogs, homologues, congeners, derivatives, salts and combinations thereof. Antiallergic agents include, but are not limited to, pemirolast potassium (ALAMAST, Santen, Inc.), and any prodrugs, codrugs, metabolites, analogs, homologues, congeners, derivatives, salts and combinations thereof. One of skill will appreciate that, in some embodiments, chemotherapy drugs can be most effective when given in combination, as a “combination chemotherapy” regime.

The chemotherapy used, for example, can be chosen to treat the particular type of cancer. In some embodiments, where the subject has a glioblastoma, temozolomide can be administered with the cancer stem cells. For example, Cancer stem cells can express the cytokine IL-33, which is known to induce resistance to chemotherapy. The use of temozolomide in the treatment of glioblastomas, for example, is blocked by IL-33, as IL-33 prevents cancer cell death by temozolomide. As such, a combination therapy of an administration of the cancer stem cells taught herein with temozolomide can be a combination of choice for treating a glioblastoma. In some embodiments, where the subject has a breast cancer, paclitaxel can be administered with the cancer stem cells. In some embodiments, the second therapeutic agent can be dacarbazine, a BRAF inhibitor and or CTLA-4, PD1 or PD-L1 blockade to treat melanoma. In some embodiments, the second therapeutic agent can be taxol, doxorubicin, vinorelbine, cyclophosphamide and/or gemcitabine to treat breast cancer. In some embodiments, the second therapeutic agent can be 5-fluorouracil and optionally leucovorin, irinoteacan and/or oxaliplatin to treat colorectal cancer. In some embodiments, the second therapeutic agent can be taxol, carboplatin, vinorelbine and/or gemcitabine, PD-1 or PD-L1 blockade to treat lung cancer. And, in some embodiments, the second therapeutic agent can be cisplatin and/or radiotherapy to treat head and neck cancer.

In some embodiments, the second therapeutic agent is selected from the group consisting of cyclophosmamide, alkylating-like agents such as cisplatin or melphalan, plant alkaloids and terpenoids such as vincristine or paclitaxel (Taxol), antimetabolites such as 5-fluorouracil, topoisomerase inhibitors type I or II such as camptothecin or doxorubicin, cytotoxic antibiotics such as actinomycin, anthracyclines such as epirubicin, glucocorticoids such as triamcinolone, inhibitors of protein, DNA and/or RNA synthesis such as methotrexate and dacarbaxine, histone deacetylase (HDAC) inhibitors.

### Radiation Therapy

The cancer stem cells taught herein can be used in combination with a radiation therapy administered as a secondary therapeutic agent. In some embodiments, the radiation therapy is used as a palliative treatment to control the rate of growth or the symptoms of the cancer where it is not possible to effectively stop the cancer, or the attempts to stop the cancer would bring the quality of life of the subject to question. In some embodiments, the radiation therapy is administered as a therapeutic treatment (where the therapy has survival benefit and can be curative).

In some embodiments, the radiation therapy can be combined with any other treatment described herein, including surgery, chemotherapy, hormone therapy, immunotherapy, targeted therapy, or a combination thereof. Any cancer taught herein can be treated with the combination of cancer stem cells and radiation therapy. One of skill will appreciate that the precise treatment used on a subject (curative, adjuvant, neoadjuvant therapeutic, or palliative) will depend on the tumor type, location, and stage, as well as the general health of the patient. In some embodiments, radiation therapy can be applied after the cancer stem cells are administered, for example, without hindering the efficacy of treatment provided by the cancer stem cells.

### Oncolytic Viruses

The cancer stem cells taught herein can be used in combination with an oncolytic virus administered as a secondary therapeutic agent. An oncolytic virus infects and replicates in tumor cells, such that the tumor cells are killed. As such, the oncolytic viruses used herein are replication competent but their replication is specific to tumor cells. The virus is selectively replication competent in tumor tissue, which means that it replicates more effectively in tumor tissue than in non-tumor tissue, or only the tumor tissue. In some embodiments, the oncolytic virus is engineered to be tumor-specific. In some embodiments, the oncolytic virus is engineered to be tumor-specific and express a desired protein. In some embodiments, the desired protein is an agent for treating, preventing, curing, at least inhibiting the onset of, or ameliorating the symptoms of, cancer in a subject.

### A Tumor-Specific Oncolytic Virus

It's beneficial to engineer the oncolytic virus to survive only if it has access to tumor tissue, because if the virus dies as the tumor tissue dies during treatment by the virus in a subject, the subject is exposed to less toxicity. As such, in some embodiments, the oncolytic virus can only replicate in tumor tissue. And, in some embodiments, the oncolytic virus is genetically engineered to be tumor-specific.

The oncolytic virus can be any strain of virus that is sufficiently effective in it's oncolytic effect on tumor cells, as well as it's replication competency and selectivity in tumor tissue. The oncolytic virus can include, but is not limited to, a strain of herpes virus, pox virus, adenovirus (Ad1, Ad2, Ad3, Ad4, Ad5, Ad11, Ad35, and Ad41, or chimeric adenovirus serotypes), retrovirus, rhabdovirus, paramyxovirus, or revirus. In some embodiments, the oncolytic virus is a herpes simplex virus (HSV). In some embodiments, the virus may be a wild type virus (i.e. unaltered from the parental virus species), or with gene disruptions or gene additions. Whether the virus used is wild type, mutated, or configured with gene deletions or additions, will depend on the virus species chosen for configuration.

The term “gene” can be used to refer to a nucleotide sequence encoding a protein, i.e., the coding sequence of the gene. A gene within an oncolytic virus may be rendered functionally inactive by any suitable method, for example by deletion or substitution of all or part of the gene and/or control sequence of the gene or by insertion of one or more nucleic acids into or in place of the gene and/or the control sequence of the gene. For example, homologous recombination methods, which are standard in the art, may be used to generate an oncolytic virus taught herein. In some embodiments, deletions may remove one or more portions of the gene, the entire gene, or the entire gene and all or some of the control sequences. For example, deletion of only one nucleotide within the gene may be made, resulting in a frame shift. However, a larger deletion may be made, at least about 25% in some embodiments, or at least about 50% in some embodiments, of the total coding and/or non-coding sequence. In some embodiments, an entire gene, and optionally some of the flanking sequences, may be removed from the virus to render the gene inactive. In some embodiments, where two or more copies of the gene are a viral genome, all copies of the gene are rendered functionally inactive.

A gene may be inactivated by substituting other sequences. In some embodiments, a gene may be inactivated by substituting all or part of the endogenous gene with a heterologous gene and optionally a promoter sequence. Where no promoter sequence is substituted, the heterologous gene may be inserted such that it is controlled by the promoter of the gene being rendered non-functional. In an HSV, for example, the ICP34.5 encoding-genes are rendered non-functional by the insertion of a heterologous gene or genes and a promoter sequence or sequences operably linked thereto, and optionally other regulatory elements such as polyadenylation sequences (polyA or pA), into each the ICP34.5-encoding gene loci.

In some embodiments, the oncolytic virus can be a wild-type strain, for example a wild-type HSV1 or HSV2 strain, or a derivative thereof. A virus derivative has at least 70%, 75%, 80%, 85%, 90%, 95%, 97%, 99%, or any % therein in increments of 1%, sequence homology to the wild-type strain. In some embodiments, a wild-type strain can be, for example, a wild-type HSV1 genome or a wild-type HSV2 genome. A virus derivative has the sequence of a HSV1 or HSV2 genome modified by nucleotide substitutions. In some embodiments, for example, the virus derivative can have 1, 2, 3, 4, 5, 6, 7, 8, 9, or 10 nucleotide substitutions. In some embodiments, the virus derivative can have 10, 11, 12, 13, 14, 15, 16, 17, 18, 19, 20, 21, 22, 23, 24, or 25 nucleotide substitutions. In some embodiments, the virus derivative can have 25, 26, 27, 28, 29, 30, 31, 32, 33, 34, 35, 36, 37, 38, 39, 40, 41, 42, 43, 44, 45, 46, 47, 48, 49, or 50 nucleotide substitutions. In some embodiments, the derivative can have a range of 1-100 nucleotide substitutions, from 1-50 nucleotide substitutions, from 1-25 nucleotide substitutions, from 1-10 nucleotide substitutions, or any amount or range of substitutions therein in increments of 1 nucleotide substitution. In some embodiments, the HSV1 or HSV2 genome may be modified by one or more insertions and/or deletions and/or by an extension at either or both ends. In some embodiments, the genomes are human. In some embodiments, the genomes are murine or mouse. And, in some embodiments, the genomes are mouse, rat, humanized versions of mouse or rat, or any other preferred non-human genome.

Each virus used herein, including the HSV1 and the HSV2, can be modified to be tumor-selective. In some embodiments, the HSV can be constructed such that it does not express functional ICP34.5 to reduce pathogenicity. In some embodiments, the HSV can be constructed such that it does not express functional ICP47 to enhance viral and tumor antigen presentation by major histocompatibility complex-I (MHC-1). In some embodiments, neither ICP34.5 nor ICP47 is expressed. In some embodiments, the ICP34.5 and/or ICP47 genes can be deleted. See U.S. Pat. No. 10,570,377 (“Coffin”) and Thomas et al. Journal for ImmunoTherapy of Cancer 7:214 (2019) (“Thomas”), each of which is hereby incorporated herein by reference in its entirety. In some embodiments, the HSV can be constructed to express the US11 gene as an immediate early gene. In some embodiments, the oncolytic virus can express one or more heterologous genes. In some embodiments, the heterologous genes can be inserted, for example, where the IPC34.5 and/or ICP47 have been deleted. See, for example, Coffin at col. 5, lines 28-53, and Example 8 (teaching deletion using GFP fluorescent protein (SEQ ID NO: 123) for making GFP expressing virus plaques; selecting virus expressing GFP in the place of the IC47 region; removing GFP from selected virus using homologous recombination with the empty flanking regions; finally, selecting plaques not expressing GFP to obtain the desired virus, for example, an ICP47 deleted virus in which US11 is expressed as an IE protein as it is now under the control of the ICP47 promoter); and, Thomas at p3 of 17, col. 1, lines 21-29.

This HSV comprises one or more mutations in one or more viral genes that inhibit replication in normal tissue but still allow replication in tumors. The mutation may, for example, be a mutation that prevents the expression of functional ICP34.5, ICP6 and/or thymidine kinase by the HSV.

In some embodiments, the oncolytic virus can kill tumor cells within 72 hours, preferably within 48 hours, more preferably within 24 hours, of infection at multiplicities of infection (MOI) of ranging from 0.0001 to 0.1, from 0.0001 to 0.01, from 0.0001 to 0.001, or any amount or range therein in MOI increments of 0.00005. In some embodiments, the virus can kill a broad range of human tumor cell lines, such as 2, 3, 4, 5, 6, 7 or all of the following cell lines: HT29 (colorectal), MDA-MB-231 (breast), SK-MEL-28 (melanoma), Fadu (squamous cell carcinoma), MCF7 (breast), A549 (lung), MIAPACA-2 (pancreas), HT1080 (fibrosarcoma). In some embodiments, an oncolytic virus can kill solid tumors, including but not limited to colorectal tumor cells, prostate tumor cells, breast tumor cells, ovarian tumor cells, melanoma cells, squamous cell carcinoma cells, lung tumor cells, pancreatic tumor cells, sarcoma cells and/or fibrosarcoma cells.

In some embodiments, the ICP34.5-encoding genes are mutated to confer selective oncolytic activity on the HSV. Mutations of the ICP34.5-encoding genes that prevent the expression of functional ICP34.5 are described in Chou et al. (1990) Science 250:1262-1266, Maclean et al. (1991) J. Gen. Virol. 72:631-639, and Liu et al. (2003) Gene Therapy 10:292-303, each of which is incorporated herein by reference in it's entirety. The ICP6-encoding gene and/or thymidine kinase-encoding gene may also be inactivated, as may other genes provided that such inactivation does not prevent the virus infecting or replicating in tumors.

### Engineering Oncolytic Viruses to Enhance Performance

Modified oncolytic viruses can be engineered using methods well known in the art to enhance performance. In some embodiments, modifications can enhance killing of tumor cells. In some embodiments, the oncolytic virus can be modified to enhance the killing of tumor cells by the oncolytic virus itself. In some embodiments, the oncolytic virus can be modified to express any desired protein known by the skilled artisan to be useful in the treatment of cancer to enhance the killing of tumor cells. For example, the modified oncolytic viruses can be engineered to express any protein taught herein. In some embodiments, plasmids (for smaller viruses and single and multiple genome component RNA viruses) or BACS (for larger DNA viruses including herpes viruses) encoding the viral genome to be packaged, including any genes encoding a desired protein, such as fusogenic and/or immune stimulating molecules under appropriate regulatory control, can be constructed by standard molecular biology techniques and transfected into cancer stem cells, or permissive cells from which recombinant viruses can be recovered and then loaded into cancer stem cells.

In some embodiments, plasmids containing DNA regions flanking the intended site of insertion can be constructed, and then co-transfected into a permissive cell with viral genomic DNA, such that homologous recombination between the target insertion site flanking regions in the plasmid and the same regions in the parental virus occur. Recombinant viruses can then be selected and purified through the loss or addition of a function inserted or deleted by the plasmid used for modification, e.g. insertion or deletion of a marker gene such as GFP or lacZ from the parental virus at the intended insertion site for loading into a cancer stem cell. In some embodiments, the insertion site is the ICP34.5 locus of HSV, and the plasmid used for manipulation contains HSV sequences flanking this insertion site, between which are an expression cassette encoding a fusogenic protein and an immune stimulatory molecule. The parental firus may contain a cassette encoding GFP in place of ICP34.5 and recombinant virus plaques are selected through the loss of expression of GFP. In some embodiments, the US11 gene of HSV is also expressed as an IE gene. This may be accomplished through deletion of the ICP47-encoding region, or by other means known to the skilled artisan.

In some embodiments, fusogenic protein encoding sequences and immune stimulatory molecule encoding sequences may be inserted into the viral genome under the regulatory control of natural promoters of the oncolytic virus used, depending on the species and insertion site, or preferably under the control of heterologous promoters. Suitable heterologous promoters include mammalian promoters, such as the IEF2a promoter or the actin promoter. In some embodiments, the heterologous promoters can include strong viral promoters such as the CMV IE promoter. An example of a CMV promoter is SEQ ID NO: 117. In some embodiments, the heterologous promoters can include strong viral promoters such as the RSV LTR. An example of an RSV promoter is SEQ ID NO: 118. In some embodiments, the heterologous promoters can include strong viral promoters such as the MMLV LTR. An example of an MMLV promoter is SEQ ID NO: SEQ ID NO: 124. In some embodiments, the heterologous promoters can include strong viral promoters such as promoters derived from SV40. An example of an SV40 promoter is SEQ ID NO: 121. In some embodiments, the promoter can be an EF1α promoter. An example of an EF1α promoter is SEQ ID NO: 125. In some embodiments, each exogenous gene expressing a desired protein can be under separate promoter control. And, in some embodiments, each exogenous gene can be expressed from a single RNA transcript, which can be accomplished, for example, through insertion of an internal ribosome entry sites (IRES) between protein coding sequences.

In some embodiments, RNA derived from each promoter can be terminated using a polyadenylation sequence such as, for example, mammalian sequences including a bovine growth hormone (BGH) poly A sequence, for example SEQ ID NO: 119; a viral sequence such as the SV40 early or late polyadenylation sequence, such as the SV40 late polyA of SEQ ID NO: 120. In some embodiments, the polyA sequence can be an human growth hormone (HGH) polyA. An example of an HGH polyA is SEQ ID NO: 122. In some embodiments, the polyA sequence can be an RBG polyA. An example of an RBG polyA is SEQ ID NO: 122.

In some embodiments, the oncolytic virus is a pox virus or a HSV, such as HSV1, which expresses at least three heterologous genes. Each of the three heterologous genes can be driven by a promoter independently selected from the CMV promoter, the RSV promoter, the EF1a promoter, the SV40 promoter and a retroviral LTR promoter. In some embodiments, the oncolytic virus can express four heterologous genes, each driven by a promoter independently selected from the CMV promoter, the RSV promoter, the EF1a promoter, the SV40 promoter and a retroviral LTR promoter. The retroviral LTR is preferably from MMLV also known as MoMuLV. In some embodiments, each of the heterologous genes may be terminated by independently selected polyadenylation sequences, meaning the poly A sequences can be the same or different among the heterologous genes. In some embodiments, each heterologous gene is terminated by the same poly A sequence. In some embodiments, each heterologous is terminated by a different polyadenylation sequence. In some embodiments, each heterologous gene is terminated by an independently selected polyadenylation sequence selected from the group consisting of BGH, SV40, HGH, and RBG poly adenylation sequences.

An oncolytic virus can be engineered to contain one or more mutations which enhance replication of the virus in tumors, increasing the rate of replication and amount of heterologous gene expression makes the oncolytic more deadly to cancer cells. In some embodiments, the enhancement can also increase the amount of tumor antigen released as tumor cells die, further improving the immunogenic properties of the therapy for the treatment of cancer. In some embodiments, in HSV, deletion of the ICP47-encoding gene can be done to place the US11 gene under the control of the immediate early promoter that normally controls expression of the ICP47 encoding gene leads to enhanced replication in tumors. See, for example, Liu et al., 2003, which is incorporated herein by reference in it's entirety.

In some embodiments, other mutations that place the US11 coding sequence, which is an HSV late gene, under the control of a promoter that is not dependent on viral replication may also be introduced into a virus of the invention. Such mutations allow expression of US11 before HSV replication occurs and enhance viral replication in tumors. In particular, such mutations enhance replication of an HSV lacking functional ICP34.5-encoding genes. As such, in some embodiments, the HSV can be configured to include a US11 gene operably linked to a promoter, wherein the activity of the promoter is not dependent on viral replication. The promoter may be an immediate early (IE) promoter or a non-HSV promoter which is active in mammalian, preferably human, tumor cells. The promoter may, for example, be a eukaryotic promoter, such as a promoter derived from the genome of a mammal, preferably a human. The promoter may be a ubiquitous promoter (such as a promoter of (β-actin or tubulin) or a cell-specific promoter, such as tumor-specific promoter. The promoter may be a viral promoter, such as the Moloney murine leukaemia virus long terminal repeat (MMLV LTR) promoter or the human or mouse cytomegalovirus (CMV) IE promoter. HSV immediate early (IE) promoters are well known in the art. In some embodiments, the HSV IE promoter may be, for example, the promoter driving expression of ICP0, ICP4, ICP22, ICP27 or ICP47.

In some embodiments, a recombinant gene encoding a desired protein contains (i) nucleic acids encoding the protein along with (ii) regulatory elements for facilitating protein expression. Generally, the regulatory elements that are present in a recombinant gene include a transcriptional promoter, a ribosome binding site, and a terminator. A promoter can be defined as a DNA sequence that directs RNA polymerase to bind to DNA and initiate RNA synthesis. A strong promoter can be one which causes mRNAs to be initiated at high frequency. A suitable element for processing expression of the protein in cells is a polyadenylation signal. Antibody-associated introns may also be present. Examples of expression cassettes for antibody or antibody fragment production are well known in art. (E.g., Persic et al., 1997, Gene 187:9-18; Boel et al., 2000, J Immunol. Methods 239:153-166; Liang et al., 2001, J. Immunol. Methods 247:1 19-130; Tsurushita et al., 2005, Methods 36:69-83.)

In some embodiments, the expression vectors can be in the form of plasmids. The terms “plasmid” and “vector” can be used interchangeably, in some embodiments. Consistent with the teachings herein, other forms of expression vectors that are not technically plasmids can be used, of course such as viral vectors. The viral vectors can include, but are not limited to, HSV, vaccina virus, replication defective retroviruses, adenoviruses and adeno-associated viruses. Such viral vectors permit infection of the cancer stem cells and expression of the desired protein. Suitably, the expression control sequences are promoter systems capable of transforming or transfecting the cancer stem cells. Once the vector has been incorporated into the appropriate host, the host is maintained under conditions suitable for high level expression of the nucleotide sequences encoding the desired protein.

In some embodiments, recombinant expression vectors can be prepared using standard recombinant DNA techniques. In some embodiments, vectors can contain regulatory sequences, such as transcription and translation initiation and termination codons, which are specific to the host cell into which the vector is to be introduced, as appropriate and taking into consideration whether the vector is DNA- or RNA-based. In some embodiments, the vector can contain a nonnative promoter operably linked to the nucleotide sequence encoding the desired protein to be expressed. In some embodiments, the promoter can be a non-viral promoter or a viral promoter, such as a cytomegalovirus (CMV) promoter, an SV40 promoter, or an RSV promoter. Other functional promoters can be identified and used by those of ordinary skill in the art.

One of skill will appreciate that an oncolytic virus can be engineered to express a desired protein. In some embodiments, the desired protein is a fusogenic protein and/or an immune stimulatory protein for use in treating cancer in a subject. A heterologous gene can be inserted for encoding the fusogenic protein and/or a heterologous gene encoding the immune stimulatory protein in the genome of a selectively replication competent virus wherein each gene is under the control of a promoter sequence. Genetic engineering will ensure that replication of such a virus will occur selectively in tumor tissue, so expression of the fusogenic protein and/or immune stimulatory protein by the virus is also enhanced by ensuring that the virus expresses selectively in tumor tissue as compared to non-tumor tissue. In some embodiments, an enhanced expression occurs through enhanced selectivity, where expression is selectively greater in tumors as compared to other tissues of the body. In some embodiments, the measure is that expression of the oncolytic virus occurs only in tumor tissue.

The fusogenic protein expressed by an oncolytic virus can be any fusogenic protein taught herein. In some embodiments, the fusogenic protein is a heterologous protein that promotes fusion of a cell infected with the virus of the invention to another cell. The oncolytic virus is engineered to carry a fusogenic gene that expresses a respective fusogenic protein. The fusogenic protein can be a wild type protein, or a modified viral glycoprotein modified to increase the fusogenic properties of the protein, inducing cell-to-cell fusion (syncitia formation) of cells in which it is expressed. In some embodiments, the fusogenic protein can be a glycoprotein selected from a group consisting of VSV-G, syncitin-1 (from human endogenous retrovirus-W (HERV-V)) or syncitin-2 (from HERVFRDE1), paramyxovirus SV5-F, measles virus-H, measles virus-F, RSV-F, the glycoprotein from a retrovirus or lentivirus, such as gibbon ape leukemia virus (GALV), murine leukemia virus (MLV), Mason-Pfizer monkey virus (MPMV), and equine infectious anemia virus (EIAV) with the R transmembrane peptide removed (R-versions). In some embodiments, the fusogenic protein is GALV having the R-peptide removed (GALV-R−).

It should be appreciated that an oncolytic virus can also be engineered to express an immunomodulatory protein using the methods taught herein. The immunomodulatory protein can be any immunomodulatory protein taught herein, and can facilitate the inducement of an immune response, reducing the inhibitory signals that lower the induction or effectiveness of an immune response. In some embodiments, the immunomodulatory protein is selected from the group consisting of IL-2, IL12, IL-15, IL-18, IL-21, IL-24, CD40 ligand, GITR ligand, 4-1-BB ligand, OX40 ligand, ICOS ligand, fit3 ligand, type I interferons, including interferon alpha and interferon beta, interferon gamma, type III interferon (IL-28, IL-29), and other cytokines such as TNF alpha or GM-CSF, TGF beta or immune checkpoint antagonists.

The immunomodulatory protein expressed can be an agonist of immune potentiating/co-stimulatory pathways. In some embodiments, the immunomodulatory can include mutant or wild type, soluble, secreted and/or membrane bound ligands, and agonistic antibodies including single chain antibodies. In some embodiments, the immunomodulatory protein targets immune co-inhibitory or immune co-stimulatory pathways, and is selected from a group consisting of proteins or other molecules (agonistic or antagonistic depending on the case) targeting CTLA-4 (antagonist), PD-1 (antagonist), PD-L1 (antagonist), LAG-3 (antagonist), TIM-3 (antagonist), VISTA (antagonist), CSF1R (antagonist), IDO (antagonist), CEACAM1 (antagonist), GITR (agonist), 4-1-BB (agonist), KIR (antagonist), SLAMF7 (antagonist), OX40 (agonist), CD40 (agonist), ICOS (agonist) and CD47 (antagonist). As such, in some embodiments, oncolytic viruses are engineered to encode one or more of these molecules. In some embodiments, the oncolytic virus encodes an immunomodulatory protein selected from the group consisting of GM-CSF and/or a wild type or modified version of CD40L, ICOSL, 4-1-BBL, GITRL, and OX40L. And, in some embodiments, the oncolytic virus encodes for expression of GM-CSF.

As such, in some embodiments, the oncolytic virus can be engineered to encode for a CTLA-4 inhibitor that binds to CTLA-4 and reduces or blocks signaling through CTLA-4, such as by reducing activation by B7, to reduce or remove the blocking of immune stimulatory pathways by CTLA-4. The CTLA-4 inhibitor is preferably an antibody or an antigen binding fragment thereof. The term “antibody” as referred to herein includes whole antibodies and any antigen binding fragment (i.e., “antigen-binding portion”) or single chains thereof. An antibody refers to a glycoprotein comprising at least two heavy (H) chains and two light (kappa)(L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g., effector cells) and the first component (Clq) of the classical complement system.

One of skill will appreciate that the oncolytic viruses used to infect the cancer stem cells can be engineered to express more than one type of desired protein. In some embodiments, the oncolytic virus is engineered to express 1 desired protein, 2 desired proteins, or 3 desired proteins. As such, in some embodiments, the oncolytic virus is engineered to have a combination of a gene expressing a fusogenic protein and a gene expressing an immunomodulatory protein, for example. Any combination of 1, 2, or 3 proteins taught herein can be expressed by an engineered oncolytic virus taught herein. In some embodiments, the cancer stem cell carries an HSV virus that expresses 1, 2, or 3 immunomodulatory cytokines. In some embodiments, the cancer stem cell carries an HSV virus that expresses IL-2 and IL-12.

For example, the oncolytic virus can be engineered to express an antibody. In some embodiments, the antibody is a monoclonal antibody. In some embodiments, the antibody is a chimeric antibody. In some embodiments, the antibody is a human antibody. And, in some embodiments, the antibody is a humanized antibody.

One of skill will appreciate having a cancer stem cell delivery system for delivering an oncolytic virus that can be engineered to express any protein taught herein as an agent for treating cancer. A protein expressed by an oncolytic virus can be engineered to be identical to a naturally occurring protein, substantially identical to a naturally occurring protein, or it may be engineered to be a modified protein to increase expression of the protein, activity of the protein, stability of the protein, or efficacy of the protein. For example, such a modified protein can be expressed from a gene modified by codon optimization. As such, the desired proteins expressed by an oncolytic virus can be mutants, variants of the wild-type protein, and the expression can be from a codon optimized nucleotide sequence. One of skill will appreciate that it is common for the skilled artisan to modify sequences with codon optimization, which is a gene engineering approach that uses synonymous codon changes to increase protein production. For example, a lead scientist can supply the nucleotide of interest to a technician, and the technician can use software algorithms for codon optimization to accommodate for codon bias, which accounts for the preference of one codon over another for the same amino acid. Applications for codon-optimization include recombinant protein drugs and nucleic acid therapies, including gene therapy, mRNA therapy, and DNA/RNA vaccines. The art offers several codon optimization tools to improve expression efficiency through synthetic gene design that provides improved sequence options using tools such as GENEWIZ (available from Azenta Life Sciences, Burlington, MA), https://www.qenewiz.com/en/Public/Services/Gene-Synthesis/Codon-Optimization?sc_device=Mobile (downloaded Jan. 10, 2023); CODON OPTIMIZATION TOOL (available from Integrated DNA Technologies, Coralville, Iowa), https://www.idtdna.com/pages/tools/codon-optimization-tool (downloaded Jan. 10, 2023); and GENSMART (available from GenScript, Piscataway, NJ)

One of skill will appreciate that the amino acid sequences can have some variation too, and still function as desired. The term “variant” refers to modifications to a protein that allows the protein to retain or improve its binding properties, and such modifications include, but are not limited to, conservative substitutions in which one or more amino acids are substituted for other amino acids; deletion or addition of amino acids that have minimal influence on the binding properties or secondary structure; post-translational modifications such as, for example, the addition of functional groups; and, the like. The term “conservatively modified variant” refers to a conservative amino acid substitution, which is an amino acid substituted by an amino acid of similar charge density, hydrophilicity/hydrophobicity, size, and/or configuration such as, for example, substituting valine for isoleucine. In comparison, a “non-conservatively modified variant” refers to a non-conservative amino acid substitution, which is an amino acid substituted by an amino acid of differing charge density, hydrophilicity/hydrophobicity, size, and/or configuration such as, for example, substituting valine for phenyalanine. In some embodiments, a protein expressed by an oncolytic virus can be a conservatively modified variant of a naturally occurring protein, a conservatively modified variant of a modified protein, or a conservatively modified variant of a recombinant protein. In some embodiments, a protein expressed by an oncolytic virus can be a non-conservatively modified variant of a naturally occurring protein, a non-conservatively modified variant of a modified protein, or a non-conservatively modified variant of a recombinant protein. Examples of conservative and non-conservative amino acid substitutions are provided in the following table and, although are non-limiting, may be used in some embodiments:

TABLE 2

| | | | non-conservative |
|---|---|---|---|
| amino acid | code | conservative substitute | substitute |
| alanine | A | D, E, G, S, T | P, V |
| cysteine | C | G, R, S, W, Y | F |
| aspartic acid | D | A, E, G, H, N, V, Y | |
| glutamic acid | E | A, D, G, K, Q, V | |
| phenylalanine | F | I, L, Y | C, S, V |
| glycine | G | A, C, D, E, R | S, V, W |
| histidine | H | D, L, N, P, Q, R, Y | |
| isoleucine | I | F, L, M, N, V | K, R, S, T |
| lysine | K | E, M, N, Q, R, T | I |
| leucine | L | F, H, I, M, P, Q, R, V, W | S |
| methionine | M | I, K, L, R, T, V | |
| asparagine | N | D, H, I, K, S, T, Y | |
| proline | P | H, L, Q, R, S | A, T |
| glutamine | Q | E, H, K, L, P, R | |
| arginine | R | C, G, H, K, L, M, P, Q, T, W | I, S |
| serine | S | A, C, N, P, T, W, Y | F, G, I, L, R |
| threonine | T | A, K, M, N, R, S | I, P |
| valine | V | D, E, I, L, M, | A, F, G |
| tryptophan | W | C, L, R, S | G |
| tyrosine | Y | C, D, F, H, N, S | |

Engineering the construct of the oncolytic viruses can be done using any of the methods known to those of skill. For example, DNA encoding the proteins may be obtained from a cDNA library prepared from tissue possessing the mRNA for the mutants. As such, the DNA can be conveniently obtained from a cDNA library prepared from human tissue. The encoding gene for the mutants may also be obtained from a genomic library or by known synthetic procedures (e.g., automated nucleic acid synthesis).

Libraries can be screened with probes (such as antibodies to the mutant P2G of human stromal cell derived factor 1 alpha or oligonucleotides of at least about 20-80 bases) designed to identify the gene of interest or the protein encoded by it. Screening the cDNA or genomic library with the selected probe may be conducted using standard hybridization procedures, such as described in Sambrook et al., Molecular Cloning: A Laboratory Manual (New York: Cold Spring Harbor Laboratory Press, 1989), which is herein incorporated by reference. An alternative means to isolate the gene encoding a protein is to use PCR methodology [Sambrook et al., supra; Dieffenbach et al., PCR Primer A Laboratory Manual (Cold Spring Harbor Laboratory Press, 1995)].

Nucleic acids having a desired protein coding sequence may be obtained by screening selected cDNA or genomic libraries using a deduced amino acid sequence and, if necessary, a conventional primer extension procedure as described in Sambrook et al., supra, to detect precursors and processing intermediates of mRNA that may not have been reverse-transcribed into cDNA.

In some embodiments, an isolated nucleotide sequence will be hybridizable, under moderately stringent conditions, to a nucleic acid having a nucleotide sequence comprising or complementary to the desired nucleotide sequences. In some embodiments, an isolated nucleotide sequence will hybridizable, under stringent conditions, to a nucleic acid having a nucleotide sequence comprising or complementary to the desired nucleotide sequences. A nucleic acid molecule is “hybridizable” to another nucleic acid molecule when a single-stranded form of the nucleic acid molecule can anneal to the other nucleic acid molecule under the appropriate conditions of temperature and ionic strength (see Sambrook et al., supra,). The conditions of temperature and ionic strength determine the “stringency” of-the hybridization. “Hybridization” requires that two nucleic acids contain complementary sequences. However, depending on the stringency of the hybridization, mismatches between bases may occur. The appropriate stringency for hybridizing nucleic acids depends on the length of the nucleic acids and the degree of complementation. Such variables are well known in the art. More specifically, the greater the degree of similarity or homology between two nucleotide sequences, the greater the value of Tm for hybrids of nucleic acids having those sequences. For hybrids of greater than 100 nucleotides in length, equations for calculating Tm have been derived (see Sambrook et al., supra). For hybridization with shorter nucleic acids, the position of mismatches becomes more important, and the length of the oligonucleotide determines its specificity (see Sambrook et al., supra).

The terms “homology” and “homologous” can be used interchangeably in some embodiments. The terms can refer to nucleic acid sequence matching and the degree to which changes in the nucleotide bases between polynucleotide sequences affects the gene expression. These terms also refer to modifications, such as deletion or insertion of one or more nucleotides, and the effects of those modifications on the functional properties of the resulting polynucleotide relative to the unmodified polynucleotide. Likewise the terms refer to polypeptide sequence matching and the degree to which changes in the polypeptide sequences, such as those seen when comparing the modified polypeptides to the unmodified polypeptide, affect the function of the polypeptide. It should be appreciated to one of skill that the polypeptides, such as the mutants taught herein, can be produced from two non-homologous polynucleotide sequences within the limits of degeneracy.

In some embodiments, the polynucleotides selected for encoding a desired protein is at least 80, 85, 90, or 95 percent homologous to the desired polynucleotide or any degenerate form of the desired polynucleotide. In some embodiments, the homology is 80%, 81%, 82%, 83%, 84%, 85%, 86%, 87%, 88%, 89%, 90%, 91%, 92%, 93%, 94%, 95%, 96%, 97%, 98%, 99%, 100%, or any amount or range therein in increments of 0.1%. One of skill will appreciate that the percent homology is determined by hybridization under varying degrees of stringency. The temperature and salt concentrations at which we perform a hybridization has a direct effect upon the results that are obtained. Specifically, you can set the conditions up so that your hybridizations only occur between the probe and a filter bound nucleic acid that is highly homologous to that probe. You can also adjust the conditions the hybridization is to a nucleic acid that has a lower degree of homology to the probe.

In some embodiments, the protein selected for expression is at least 80, 85, 90, or 95 percent homologous to the desired protein. In some embodiments, the protein expressed is at least 85, 90, or 95 percent homologous to the desired protein and binds to a desired receptor. In some embodiments, the homology is 80%, 81%, 82%, 83%, 84%, 85%, 86%, 87%, 88%, 89%, 90%, 91%, 92%, 93%, 94%, 95%, 96%, 97%, 98%, 99%, 100%, or any amount or range therein in increments of 0.1%. In some embodiments, the protein expressed can be 90%, 91%, 92%, 93%, 94%, 95%, 96%, 97%, 98%, 99% homologous to an immunomodulatory cytokine taught herein, whether wild-type or variant. One of skill will appreciate that any of the several software programs for searching for amino acid sequence homology can be used, like BLAST, PSI-BLAST, SSEARCH, FASTA, and HMMER3, providing accurate statistical estimates, ensuring protein sequences that share significant similarity.

The selection of expression vectors, control sequences, transformation methods, and the like, are dependent on the type of host cell used to express the gene. Following entry into a cell, all or part of the vector DNA, including the insert DNA, may be incorporated into the host cell chromosome, or the vector may be maintained extrachromosomally. Vectors that are maintained extrachromosomally are frequently capable of autonomous replication in the host cell. Other vectors are integrated into the genome of a host cell upon and are replicated along with the host genome. The nucleotides selected for expressing the proteins can be wild-type or mutants.

In some embodiments, the oncolytic virus is an HSV virus. In some embodiments, the oncolytic virus is an HSV1 virus. In some embodiments, the oncolytic virus is an HSV2 virus. In some embodiments, the oncolytic virus is an HSV1 virus selected from the group consisting of
strain RH018A having the provisional accession number ECCAC 16121904;
strain RH004A having the provisional accession number ECCAC 16121902;
strain RH031A having the provisional accession number ECCAC 16121907;
strain RH040A having the provisional accession number ECCAC 16121908;
strain RH015A having the provisional accession number ECCAC 16121903;
strain RH021A having the provisional accession number ECCAC 16121905;
strain RH023A having the provisional accession number ECCAC 16121906; and,
strain RH047A having the provisional accession number ECCAC 16121909.
   Loading Cancer Stem Cells with an Oncolytic Virus

Methods of loading the cancer stem cells by an oncolytic virus are known to the ordinarily skilled artisan. The terms “loading” and “infecting” can be used synonymously in some embodiments, and the methods of infecting the cancer stem cells can include, for example, the use of liposomes, electroporation, microinjection, cell fusion, polymer-based systems, DEAE-dextran, viral transduction, the calcium phosphate precipitation method, etc. Any known method of loading a cell with a virus can be used. In some embodiments, natural polymer-based delivery vehicles can be used, such as chitosan and gelatin, in addition to viral vectors. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). In some embodiments, the methods of loading the cancer stem cells can include, for example, CaCl2, CaPO4, liposome-mediated transfection and electroporation. Transformation is performed using standard techniques known to the skilled artisan to be appropriate for the cancer stem cells. The calcium treatment employing calcium chloride is described in Sambrook et al., supra. In some embodiments, the calcium phosphate precipitation method of Graham and van der Eb, Virology, 52:456 457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyomithine, may also be used. For various techniques for transforming mammalian cells, see Keown et al., Methods in Enzymology, 185:527 537 (1990) and Mansour et al., Nature, 336:348 352 (1988).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486,1981), lambda (Nash, Nature, 247, 543-545, 1974), Flp (Broach, et al., Cell, 29:227-234, 1982), R (Matsuzaki, et al., J. Bacteriology, 172:610-618,1990), cpC31 (see, e.g., Groth et al., J. Mol. Biol. 335:667-678, 2004), sleeping beauty, transposases of the mariner family (Plasterk et al., supra), and components for integrating the viruses that include the LTR sequences of retroviruses or lentivirus and the ITR sequences of AAV (Kootstra et al., Ann. Rev. Pharm. Toxicol., 43:413-439, 2003).

A Triple-Killer Delivery System is Provided Using the Cancer Stem Cells as an Antigenic Delivery Vehicle for an Oncolytic Virus that Expresses a Desired Protein

The oncolytic viruses taught herein can be delivered by the programmed cancer stem cells to treat cancer in a subject, and the cells can be personalized to the subject receiving the treatment or from a donor, for example.

In some embodiments, the cancer stem cells are loaded with an oncolytic virus taught herein for delivery of the oncolytic virus to cancer tissue to treat the cancer. In some embodiments, the loaded cancer stem cells can be administered to obtain a two-prong killer “dual killer” effect where (1) the oncolytic virus kills infected tumor cells by virus-mediated toxicity, including by lysis, necrosis or apoptosis; and, (2) the cancer stem cells and the oncolytic virus delivered by the stem cells generate an immune response. And, in some embodiments, the loaded cancer stem cells can be administered to obtain a three-prong “triple-killer” effect where (1) the oncolytic virus kills infected tumor cells by virus-mediated toxicity, including by lysis, necrosis or apoptosis; (2) the cancer stem cells and the oncolytic virus delivered by the stem cells generate an immune response; and (3) the expression of a desired protein or combination of proteins from the oncolytic virus further kill cancer cells.

The delivery of the oncolytic virus comprises administering the loaded cancer stem cells that are programmed to die, and the cancer stem cells release a therapeutically effective amount of the oncolytic virus. In some embodiments, the oncolytic virus also expresses a desired protein, including a cytoxic protein, an immunomodulatory protein, a fusogenic protein, or a combination thereof. A desired protein can be any protein known by the skilled artisan to have a desired therapeutic effect, and the nucleic acid encoding the desired protein can be readily determined by the skilled artisan within the limits of degeneracy. The skilled artisan can design the oncolytic virus to express the desired protein using any genetic engineering methods known in the art. In some embodiments, the desired protein is any protein taught herein to be a therapeutic agent used to inhibit the onset of, treat, prevent, cure, or at least ameliorate the symptoms of, cancer. And, in some embodiments, the nucleic acid encoding the desired protein is also taught herein.

One of skill in the art will appreciate that the cancer stem cell drug delivery system is configured to (i) provide a high amount of protection against the development of a cancer, (ii) provide a long-term survival of the subject treated, (iii) does not require the use of dendritic cells, (iv) increase the half-life of the virus through the cancer stem cell delivery mechanism, (v) get through the blood-brain barrier to treat brain tumors, (vi) show no obvious toxicity, (vii) act as a dual killer, releasing 2 therapeutic attacks that include death to the cancer through a cancer antigen cocktail created by the cancer stem cells, and death to the cancer through the oncolytic virus; and (viii) act as a triple-killer, releasing 3 therapeutic attacks that include
death to the cancer through a cancer antigen cocktail created by the cancer stem cell;
death to the cancer through the oncolytic virus; and,
death to the cancer through release of a desired protein, for example, the release of an essential immune cytokine.

In addition, in some embodiments, the treatments can also provide a sustained immunological memory response to provide a sustained death to the cancer.

Moreover, the cancer stem cell provides a protective packaging for the oncolytic virus during delivery. So, not only does the cancer stem cell provide a place for amplification of the oncolytic virus during delivery, but the cancer stem cell also provides a protective packaging that offers the following benefits:
avoids the toxicity to the subject associated with a pure administration of the oncolytic virus itself, or the expressed protein itself, when compared to state-of-the-art IV, IM, or subcutaneous deliveries; and,
avoids the attack by the immune system associated with a pure administration of the oncolytic virus itself, or the expressed protein itself, resulting in an increased half-life of the expressed protein when compared traditional delivery methods including state-of-the-art IV, IM, or subcutaneous deliveries of the oncolytic virus itself, or the expressed protein itself.

Since the cancer stem cells can be taken from the subject receiving the treatment, the drug delivery systems taught herein can provide personalized cancer treatments, whether the vaccine, the two-prong “dual killer”, or the three-prong “triple killer”, each of which is quite valuable to any patient at risk of, or suffering, cancer. It should be appreciated that the delivery systems and methods taught herein provide a high treatment, cure, or prevention value is offered to those having a significant family history of cancer, a high risk of genetic mutations, or the presence of early stage of cancers, as the systems provide a powerful and effective therapy for later stage cancers while avoiding the toxicity problems currently suffered by the art.

### Methods of Administration

The programmed cancer stem cells can be administered as a primary therapeutic agent as taught herein. Likewise, any secondary therapeutic agent taught herein can be administered to a subject to accompany the programmed cancer stem cells. Any administration route that is known to be suitable by the skilled artisan, whether systemic or local can be used, whether it be an administration of the cancer stem cells alone, the cancer stem cells loaded with an oncolytic virus, the cancer stem cells loaded with an oncolytic virus expressing a desired protein, as a primary therapeutic agent, or the administration of a secondary therapeutic agent.

As such, the programmed cancer stem cells or agents may be administered using standard administration techniques, formulations, and/or devices. Formulations and devices, such as syringes and vials, for storage and administration can be used. With respect to cells, administration can be autologous or heterologous, in some embodiments. For example, cancer stem cells can be obtained from a subject, programmed and optionally infected with an oncolytic virus, the oncolytic virus optionally engineered to express a desired protein, and administered to the same subject for a personalized therapy, or to a different, compatible subject. In some embodiments, cancer stem cells can be administered via localized injection, including catheter administration, systemic injection, localized injection, intravenous injection, or parenteral administration. In some embodiments, a unit dosage form can be formulated in an injectable form for administration as a solution, suspension, or emulsion.

In some embodiments, an agent can be administered by intra-tumoral injection, perhaps including use of imaging guide the administration to target the tumor or tumors. In some embodiments, an agent can be administered into a body cavity, for example into the pleural cavity, bladder or by intra-peritoneal administration. In some embodiments, an agent can be injected into a blood vessel, for example, a blood vessel supplying a tumor.

In some embodiments, an agent can be administered as an injectable solution, suspension or emulsion. In some embodiments, an agent can be administered by parenteral, subcutaneous, oral, epidermal, intradermal, intramuscular, interarterial, intraperitoneal, or intravenous injection. In some embodiments, an agent can be administered topically to skin or mucosal tissue, whether nasally, intratrachealy, intestinally, sublingually, rectally or vaginally. In some embodiments, an agent can be administered as a spray suitable for respiratory or pulmonary administration.

One of skill understands that the amount of the agents administered can vary according to factors such as, for example, the type of disease, age, sex, and weight of the subject, as well as the method of administration. For example, local and systemic administration can call for substantially different amounts to be effective. Dosage regimens may also be adjusted to optimize a therapeutic response. In some embodiments, a single bolus may be administered; several divided doses may be administered over time; the dose may be proportionally reduced or increased; or, any combination thereof, as indicated by the exigencies of the therapeutic situation and factors known one of skill in the art. It is to be noted that dosage values may vary with the severity of the condition to be alleviated. Dosage regimens may be adjusted over time according to the individual need and the professional judgment of the person administering or supervising the administration of the compositions, and the dosage ranges set forth herein are exemplary only and do not limit the dosage ranges that may be selected by medical practitioners. The administration methods can provide a therapeutic and/or prophylactic effect in the treatment of a disease, or ameliorization of one or more symptoms of a disease in a subject.

The terms “administration” or “administering” refer to a method of incorporating a composition into the cells or tissues of a subject, either in vivo or ex vivo to diagnose, prevent, treat, or ameliorate a symptom of a disease. In one example, a compound can be administered to a subject in vivo parenterally. In another example, a compound can be administered to a subject by combining the compound with cell tissue from the subject ex vivo for purposes that include, but are not limited to, assays for determining utility and efficacy of a composition. When the compound is incorporated in the subject in combination with one or active agents, the terms “administration” or “administering” can include sequential or concurrent incorporation of the compound with the other agents such as, for example, any agent described above. A pharmaceutical composition of the invention is formulated to be compatible with its intended route of administration. Examples of routes of administration include, but are not limited to, parenteral such as, for example, intravenous, intradermal, intramuscular, and subcutaneous injection; oral; inhalation; intranasal; transdermal; transmucosal; and rectal administration.

The cancer stem cells, cancer stem cells loaded with oncolytic virus, cancer stem cells loaded with oncolytic virus that expresses a desired protein, and/or second therapeutic agent can be administered to a subject in a therapeutically effective amount as a therapeutic agent for treatment of a subject. The terms “therapeutic” or “treatment” can refer to an agent or method of using an agent that at least inhibits the onset of a cancer, at least inhibits the growth of cancer tissue, at least inhibits the onset of, or severity of, symptoms of the cancer, at least increases the lifespan of the patient, at least increases the quality of life of the patient, at least increases the time before a relapse, or any combination thereof. In some embodiments, “therapeutic” or “treatment” can refer to preventing the onset of the cancer, curing the subject of the cancer by killing all cancer tissue, preventing a relapse of the cancer, preventing the onset of symptoms of the cancer, or any combination thereof.

The cancers treated can be at any stage. In some embodiments, the treatments are administered to Stage I, II, III, or IV cancers. In some embodiments, the treatments are administered to Stage II, III or IV cancers. In some embodiments, the treatments are administered to Stage III or IV cancers. In some embodiments, the treatments are administered before or after surgery. In some embodiments, the surgery can be a resection of primary or recurrent/metastatic tissue. In some embodiments, the treatments are administered after surgery to treat residual tumor remains.

An “effective amount” of a compound of the invention can be used to describe a therapeutically effective amount or a prophylactically effective amount. An effective amount can also be an amount that ameliorates the symptoms of a disease. A “therapeutically effective amount” refers to an amount that is effective at the dosages and periods of time necessary to achieve a desired therapeutic result and may also refer to an amount of active compound, prodrug or pharmaceutical agent that elicits any biological or medicinal response in a tissue, system, or subject that is sought by a researcher, veterinarian, medical doctor or other clinician that may be part of a treatment plan leading to a desired effect. In some embodiments, the therapeutically effective amount may need to be administered in an amount sufficient to result in amelioration of one or more symptoms of a disorder, prevention of the advancement of a disorder, or regression of a disorder. In some embodiments, for example, a therapeutically effective amount can refer to the amount of an agent that provides a measurable response of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of a desired action of the composition. The therapeutically effective concentration of any agent, or combination of agents, taught herein may be determined empirically without undue experimentation by testing the compounds in in vitro and in vivo systems described and then extrapolated therefrom for dosages for humans. The term “treating” refers to the administering one or more therapeutic or prophylactic agents taught herein.

A “prophylactically effective amount” refers to an amount that is effective at the dosages and periods of time necessary to achieve a desired prophylactic result such as, preventing, inhibiting, or reversing angiogenesis, tumor growth, or tumor invasion. Typically, a prophylactic dose is used in a subject prior to the onset of a disease, or at an early stage of the onset of a disease, to prevent or inhibit onset of the disease or symptoms of the disease. A prophylactically effective amount may be less than, greater than, or equal to a therapeutically effective amount.

The administration of an agent taught herein, cancer stem cell or other agent, can be local or systemic. In some embodiments, the administration can be oral. In other embodiments, the administration can be subcutaneous injection. In other embodiments, the administration can be intravenous injection using a sterile isotonic aqueous buffer. In another embodiment, the administration can include a solubilizing agent and a local anesthetic such as lignocaine to ease discomfort at the site of injection. In other embodiments, the administrations may be parenteral to obtain, for example, ease and uniformity of administration.

The compounds can be administered in dosage units. The term “dosage unit” refers to discrete, predetermined quantities of a compound that can be administered as unitary dosages to a subject. A predetermined quantity of active compound can be selected to produce a desired therapeutic effect and can be administered with a pharmaceutically acceptable carrier. The predetermined quantity in each unit dosage can depend on factors that include, but are not limited to, (a) the unique characteristics of the active compound and the particular therapeutic effect to be achieved, and (b) the limitations inherent in the art of creating and administering such dosage units.

One of ordinary skill can readily select a carrier for the administration of the cancer stem cells. Sterile saline solution as a carrier, for example, does not produce any immune reaction or antitumor effect. As such, in some embodiments, a sterile saline solution can be used as a carrier for administration of the cancer stem cells. In some embodiments, a sterile phosphate buffer saline or any isotonic solution can also be used as a stem cell carrier. Carrier solutions that can be used include, for example, a carrier selected from the group consisting of 0.9% saline, phosphate-buffered solution (PBS), 5% dextrose solution, heparin in saline (Hepa-Sal) (e.g., 1 IU/mL), and Hartmann's solution.

A “pharmaceutically acceptable carrier” is a diluent, adjuvant, excipient, or vehicle with which the composition is administered. A carrier is pharmaceutically acceptable after approval by a state or federal regulatory agency or listing in the U.S. Pharmacopeial Convention or other generally recognized sources for use in subjects.

The pharmaceutical carriers include any and all physiologically compatible solvents, dispersion media, coatings, antibacterial and antifungal agents, isotonic and absorption delaying agents, and the like. Examples of pharmaceutical carriers include, but are not limited to, sterile liquids, such as water, oils and lipids such as, for example, phospholipids and glycolipids. These sterile liquids include, but are not limited to, those derived from petroleum, animal, vegetable or synthetic origin such as, for example, peanut oil, soybean oil, mineral oil, sesame oil, and the like. Water can be a preferred carrier for intravenous administration. Saline solutions, aqueous dextrose and glycerol solutions can also be liquid carriers, particularly for injectable solutions.

Suitable pharmaceutical excipients include, but are not limited to, starch, sugars, inert polymers, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain minor amounts of wetting agents, emulsifying agents, pH buffering agents, or a combination thereof. The compositions can take the form of solutions, suspensions, emulsion, tablets, pills, capsules, powders, sustained-release formulations and the like. Oral formulations can include standard carriers such as, for example, pharmaceutical grades mannitol, lactose, starch, magnesium stearate, sodium saccharine, cellulose, magnesium carbonate, and the like. See Martin, E.W. Remington's Pharmaceutical Sciences. Supplementary active compounds can also be incorporated into the compositions.

In some embodiments, the carrier is suitable for parenteral administration. In other embodiments, the carrier can be suitable for intravenous, intraperitoneal, intramuscular, sublingual or oral administration. In other embodiments, the pharmaceutically acceptable carrier may comprise pharmaceutically acceptable salts.

Pharmaceutical formulations for parenteral administration may include liposomes. Liposomes and emulsions are delivery vehicles or carriers that are especially useful for hydrophobic drugs. Depending on biological stability of the therapeutic reagent, additional strategies for protein stabilization may be employed. Furthermore, one may administer the drug in a targeted drug delivery system such as, for example, in a liposome coated with target-specific antibody. The liposomes can be designed, for example, to bind to a target protein and be taken up selectively by the cell expressing the target protein.

Therapeutic compositions typically must be sterile and stable under the conditions of manufacture and storage. The composition can be formulated as a solution, microemulsion, liposome, or other ordered structure suitable for a high drug concentration. In some embodiments, the carrier can be a solvent or dispersion medium including, but not limited to, water; ethanol; a polyol such as for example, glycerol, propylene glycol, liquid polyethylene glycol, and the like; and, combinations thereof. The proper fluidity can be maintained in a variety of ways such as, for example, using a coating such as lecithin, maintaining a required particle size in dispersions, and using surfactants.

In some embodiments, isotonic agents can be used such as, for example, sugars; polyalcohols that include, but are not limited to, mannitol, sorbitol, glycerol, and combinations thereof; and sodium chloride. Sustained absorption characteristics can be introduced into the compositions by including agents that delay absorption such as, for example, monostearate salts, gelatin, and slow release polymers. Carriers can be used to protect active compounds against rapid release, and such carriers include, but are not limited to, controlled release formulations in implants and microencapsulated delivery systems. Biodegradable and biocompatible polymers can be used such as, for example, ethylene vinyl acetate, polyanhydrides, polyglycolic acid, collagen, polyorthoesters, polylactic acid, polycaprolactone, polyglycolic copolymer (PLG), and the like. Such formulations can generally be prepared using methods known to one of skill in the art.

The compounds may be administered as suspensions such as, for example, oily suspensions for injection. Lipophilic solvents or vehicles include, but are not limited to, fatty oils such as, for example, sesame oil; synthetic fatty acid esters, such as ethyl oleate or triglycerides; and liposomes. Suspensions that can be used for injection may also contain substances that increase the viscosity of the suspension such as, for example, sodium carboxymethyl cellulose, sorbitol, or dextran. Optionally, a suspension may contain stabilizers or agents that increase the solubility of the compounds and allow for preparation of highly concentrated solutions.

In one embodiment, a sterile and injectable solution can be prepared by incorporating an effective amount of an active compound in a solvent with any one or any combination of desired additional ingredients described above, filtering, and then sterilizing the solution. In another embodiment, dispersions can be prepared by incorporating an active compound into a sterile vehicle containing a dispersion medium and any one or any combination of desired additional ingredients described above. Sterile powders can be prepared for use in sterile and injectable solutions by vacuum drying, freeze-drying, or a combination thereof, to yield a powder that can be comprised of the active ingredient and any desired additional ingredients. Moreover, the additional ingredients can be from a separately prepared sterile and filtered solution. In another embodiment, the extract may be prepared in combination with one or more additional compounds that enhance the solubility of the extract.

The amount of reprogrammed cancer stem cells desired in a single dose can vary, depending on the type of cancer, whether it is a prophylactic treatment to avoid the onset of cancer or a treatment to kill an established cancer, the stage of the cancer, and other factors such as the age and weight of the subject. In some embodiments, the amount of reprogrammed cancer stem cells desired in a single dose can range, for example, from 0.1×10⁶ to 5×10⁶ cells/kg of body weight per dose, and perhaps from 1×10¹ to 500×10⁶ cells per dose. A suggested dosing, in some embodiments, is once per week for 3 weeks, in which each dose can be administered intravenously. One of skill will appreciate that the dosing is subjective and selected according to many variables including, for example, (i) the effect sought, such as an antigenic effect of a vaccine; a prophylactic effect to prevent, or at least inhibit the onset of a cancer; or, a treatment effect of killing all cancer cells, or at least inhibiting the growth or metastasis of the cancer cells; (ii) the subject treated, such as a healthy subject or a sick subject, the subjects size and/or age, and or risk factors in the subject that include an intolerance or allergy that could elicit an undesirable immune response; (iii) the cancer treated, such as a slow-moving cancer or an aggressive cancer; and (iv) the oncolytic virus and/or desired protein administered.

In some embodiments, the amount of reprogrammed cancer stem cells desired in a single dose can range from 0.1×10⁶ to 5.0×10⁶ cells/kg of body weight per dose, from 0.2×10⁶ to 4.0×10⁶ cells/kg of body weight per dose, from 0.3×10⁶ to 3.0×10⁶ cells/kg of body weight per dose, from 0.4×10⁶ to 4.0×10⁶ cells/kg of body weight per dose, from 0.5×10⁶ to 5.0×10⁶ cells/kg of body weight per dose, or any range or amount therein in increments of 0.1×10⁶ cells/kg of body weight per dose.

In some embodiments, the amount of reprogrammed cancer stem cells desired in a single dose can range from 1×10⁶ to 50×10⁶ cells per dose, from 2×10⁶ to 40×10⁶ cells per dose, from 3×10⁶ to 30×10⁶ cells per dose, from 4×10⁶ to 20×10⁶ cells per dose, from 5×10⁶ to 50×10⁶ cells per dose, or any range or amount therein in increments of 1×10⁶ cells per dose.

In some embodiments, the amount of reprogrammed cancer stem cells desired in a single dose can range from 1×10⁶ to 500×10⁶ cells per dose, from 2×10⁶ to 400×10⁶ cells per dose, from 3×10⁶ to 300×10⁶ cells per dose, from 4×10⁶ to 200×10⁶ cells per dose, from 5×10⁶ to 100×10⁶ cells per dose, or any range or amount therein in increments of 1×10⁶ cells per dose.

In some embodiments, the amount of reprogrammed cancer stem cells desired in a single dose can be about 1×10⁶ cells per dose, about 2×10⁶ cells per dose, about 3×10⁶ cells per dose, about 4×10⁶ cells per dose, about 5×10⁶ cells per dose, about 6×10⁶ cells per dose, about 7×10⁶ cells per dose, about 8×10⁶ cells per dose, about 9×10⁶ cells per dose, about 10×10⁶ cells per dose, about 15×10⁶ cells per dose, about 20×10⁶ cells per dose, about 25×10⁶ cells per dose, about 30×10⁶ cells per dose, about 35×10⁶ cells per dose, about 40×10⁶ cells per dose, about 45×10⁶ cells per dose, about 50×10⁶ cells per dose, about 55×10⁶ cells per dose, about 60×10⁶ cells per dose, about 65×10⁶ cells per dose, about 70×10⁶ cells per dose, about 75×10⁶ cells per dose, about 80×10⁶ cells per dose, about 85×10⁶ cells per dose, about 90×10⁶ cells per dose, about 95×10⁶ cells per dose, about 100×10⁶ cells per dose, about 110×10⁶ cells per dose, about 120×10⁶ cells per dose, about 130×10⁶ cells per dose, about 140×10⁶ cells per dose, about 150×10⁶ cells per dose, about 160×10⁶ cells per dose, about 170×10⁶ cells per dose, about 180×10⁶ cells per dose, about 190×10⁶ cells per dose, about 200×10⁶ cells per dose, about 210×10⁶ cells per dose, about 220×10⁶ cells per dose, about 230×10⁶ cells per dose, about 240×10⁶ cells per dose, about 250×10⁶ cells per dose, about 260×10⁶ cells per dose, about 270×10⁶ cells per dose, about 280×10⁶ cells per dose, about 290×10⁶ cells per dose, about 300×10⁶ cells per dose, about 310×10⁶ cells per dose, about 320×10⁵ cells per dose, about 330×10⁶ cells per dose, about 340×10⁶ cells per dose, about 350×10⁶ cells per dose, about 360×10⁶ cells per dose, about 370×10⁶ cells per dose, about 380×10⁶ cells per dose, about 390×10⁶ cells per dose, about 400×10⁶ cells per dose, about 410×10⁶ cells per dose, about 420×10⁶, about 430×10⁶, about 440×10⁶, about 450×10⁶, about 460×10⁶, about 470×10⁶, about 480×10⁶, about 490×10⁶, about 500×10¹ cells per dose, or any range or amount therein in increments of 1×10¹ cells per dose.

Any number of doses can be administered to a subject as a vaccine, or for any treatment taught herein, for example, to administer an oncolytic virus via the cancer stem cells, or to administer a modified oncolytic virus to express one or more desired proteins via the cancer stem cells. In some embodiments, the cancer stem cells can be administered in 1 dose, 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 doses, 20 doses, or more, and the number of doses, and amount of live, programmed cancer stem cells administered can be independently selected to be the same or different than a previous dose during a course of administrations. The frequency of administration of doses, in some embodiments, can be 1 week apart, 2 weeks apart, 3 weeks apart, or 4 weeks apart.

In some embodiments, the amount of oncolytic virus delivered from the cancer stem cells can range from 10⁻ pfu to 10¹⁰ pfu, from 10⁵ pfu to 10⁹ pfu, from 10⁶ pfu to 10⁶ pfu, or any amount or range therein in increments of 10 pfu. In the case of HSV, the amount delivered from the cancer stem cells can range from about 10⁴ pfu to 10¹⁰ pfu, from 10⁵ pfu to 10⁹ pfu, from 10⁶ pfu to 10⁸ pfu, or any amount or range therein in increments of 10 pfu. An initial lower dose of HSV (e.g. 10⁴ to 10⁷ pfu) can be administered to seroconvert patients who are seronegative for HSV, and to boost immunity in those who are seropositive for HSV. A higher dose can then be administered in the treatment, for example, 10⁶ pfu to 10⁹ pfu). In some embodiments, the amount of oncolytic virus delivered from the cancer stem cells is adjusted by selecting an amount of virus loaded into the cancer stem cell, selecting an engineered rate of replication of the virus in the cancer stem cell, selecting a programmed time to death of the cancer stem cell, and selecting a targeted amount of the desired protein to deliver to the subject from the oncolytic virus.

The treatments may include various “unit doses”, where “unit dose” is defined as containing a predetermined-quantity of a therapeutic agent. A unit dose need not be administered as a single injection but may comprise continuous infusion over a set period of time. Plaque forming units (pfu) can be used as the unit dose for a viral construct. Unit doses ranging from 10³ pfu, 10⁴ pfu, 10⁵ pfu, 10⁶ pfu, 10⁷ pfu, 10⁸ pfu, 10⁹ pfu, 10¹⁰ pfu, 10¹¹ pfu, 10¹² pfu, 10¹³ pfu and higher can be used. Alternatively, depending on the kind of virus and the titer attainable, delivery of 1 to 100 vp, 10 to 50 vp, 10 to 500 vp, 100-1000 vp, or up to about or at least about 1×10⁴ vp, 1×10⁵ vp, 1×10⁶ vp, 1×10⁷ vp, 1×10⁶ vp, 1×10⁹ vp, 1×10¹⁰ vp, 1×10¹¹ vp, 1×10¹² vp, 1×10¹³ vp, 1×10¹⁴ vp, 1×10¹⁵ vp, or 1×10¹⁶ vp or higher infectious viral particles (vp) can be used at a tumor or tumor site, including any amount or range therein in increments of 1 vp.

The cancer stem cells, cancer stem cells loaded with an oncolytic virus, and cancer stem cells loaded with an oncolytic virus engineered to release a desired protein can be delivered as a pharmaceutical composition having a carrier. Each of the volumes containing the pharmaceutical compositions can include any cancer stem cell count taught herein, whether the count is expressed as “cells per dose” (for a 75 kg subject), or “cells/kg body weight per dose”. As such, each of the following volumes provide a cancer stem cell concentration per dose (cell count/volume), and this can convert into a concentration of oncolytic virus administered, and/or a concentration of desired protein administered. In some embodiments, the volume administered can range from about 1 ml to about 20 ml, about 2 ml to about 20 ml, about 3 ml to about 20 ml, about 4 ml to about 20 ml, about 5 ml to about 20 ml, about 6 ml to about 20 ml, about 7 ml to about 20 ml, about 8 ml to about 20 ml, about 9 ml to about 20 ml, about 10 ml to about 20 ml, about 1 ml to about 10 ml, about 2 ml to about 10 ml, about 3 ml to about 10 ml, about 4 ml to about 10 ml, about 5 ml to about 10 ml, about 6 ml to about 10 ml, about 7 ml to about 10 ml, about 8 ml to about 10 ml, about 9 ml to about 10 ml, about 11 ml to about 20 ml, about 12 ml to about 20 ml, about 13 ml to about 20 ml, about 14 ml to about 20 ml, about 15 ml to about 20 ml, about 16 ml to about 20 ml, about 17 ml to about 20 ml, about 18 ml to about 20 ml, about 19 ml to about 20 ml, or any amount or range therein in increments of 0.1 ml.

The pharmaceutical composition can be directly injected into tumors using any amount of agent or combination of agents taught herein in the volume. In some embodiments, from about 1 ml to about 50 ml, about 2 ml to about 50 ml, about 3 ml to about 50 ml, about 4 ml to about 50 ml, about 5 ml to about 50 ml, about 10 ml to about 50 ml, about 12 ml to about 50 ml, about 14 ml to about 50 ml, about 16 ml to about 50 ml, about 18 ml to about 50 ml, about 20 ml to about 50 ml, about 30 ml to about 50 ml, about 40 ml to about 50 ml, or any amount or range therein in increments of 0.1 ml of a pharmaceutical composition can be administered into a body cavity or the bloodstream any amount of agent or combination of agents taught herein in the volume. It should be appreciated that larger or smaller volumes can be used and selected by the skilled artisan, depending on the tumor and the administration route and site.

In some embodiments, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

In some embodiments, a therapeutically or prophylactically effective amount of a composition may range in concentration from about 0.001 nM to about 0.10 M; from about 0.001 nM to about 0.5 M; from about 0.01 nM to about 150 nM; from about 0.01 nM to about 500 μM; from about 0.01 nM to about 1000 nM, 0.001 μM to about 0.10 M; from about 0.001 μM to about 0.5 M; from about 0.01 μM to about 150 μM; from about 0.01 μM to about 500 μM; from about 0.01 μM to about 1000 nM, or any range therein. In some embodiments, the compositions may be administered in an amount ranging from about 0.001 mg/kg to about 500 mg/kg; from about 0.005 mg/kg to about 400 mg/kg; from about 0.01 mg/kg to about 300 mg/kg; from about 0.01 mg/kg to about 250 mg/kg; from about 0.1 mg/kg to about 200 mg/kg; from about 0.2 mg/kg to about 150 mg/kg; from about 0.4 mg/kg to about 120 mg/kg; from about 0.15 mg/kg to about 100 mg/kg, from about 0.15 mg/kg to about 50 mg/kg, from about 0.5 mg/kg to about 10 mg/kg, or any range therein, wherein a human subject is assumed to average about 70 kg.

### EXAMPLES

### Example 1. A Vaccine Having Cancer Stem Cells (Glioblastoma-Specific Stem Cells) Provided a 100% Protection from Developing a Glioblastoma in a Mouse Model

An immunocompetent orthotopic cancer-stem-cell-derived mouse glioblastoma model (the 005 GBM model) was created. The orthotopic model was developed to test the efficacy of a cancer stem cell vaccine taught herein.

A glioblastoma was induced in mice in the orthotopic model. Some of the mice received the vaccine and others received only a control, saline solution. It was demonstrated that mice receiving the vaccine were 100% protected from developing the glioblastoma and lived. Mice receiving no vaccine, merely receiving the saline, developed the glioblastoma and died.

The glioblastoma stem cells were collected from a glioblastoma brain tumor from the 005 GBM model, harvested from the solid cancer tissue, isolated, cultured, and split into a single cell suspension to create a vaccine.

The glioblastoma stem cells were reprogrammed to die to create the vaccine. The glioblastoma stem cells were irradiated with a 35 Gy dose of radiation to create dying cancer stem cells for use in the vaccine. The dying cancer stem cells remain alive until after the administration of the vaccine and are the cancer antigens in the vaccine. An MDS Nordion Gammacell Irradiator was used, irradiating the stem cells with the 35 Gy dose of gamma rays.

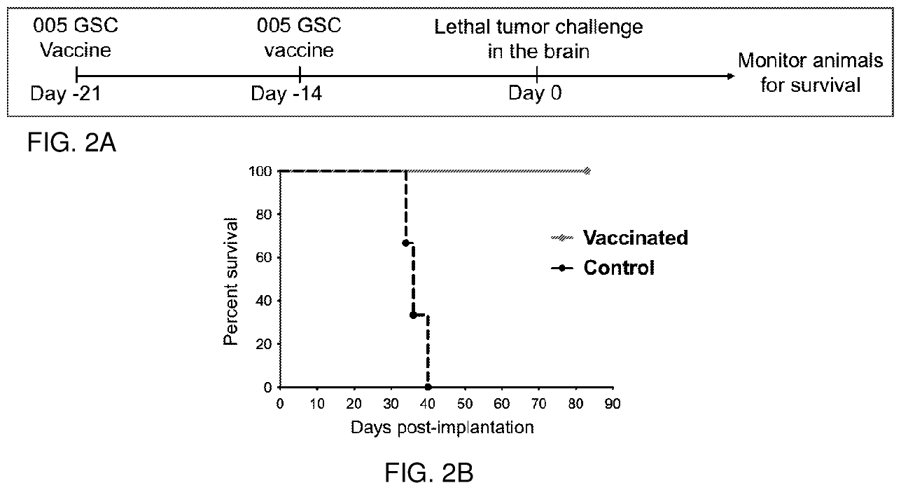

FIGS. 2A and 2B show how a cancer stem cell vaccine provided 100% protection from glioblastoma in an orthotopic glioblastoma stem-cell-derived, according to some embodiments. The model (the 005 model) uses C576BL/6 mice. FIG. 2A illustrates the treatment schedule. The C57BL/6 mice were vaccinated, meaning they were injected subcutaneously in the flank with irradiated cancer stem cells (the 005 CSCs; 1×10⁶ cells/mouse), or saline as control, on day −21 and −14. As shown in FIG. 2A, seven days after the 2nd dose of vaccination or saline injection (i.e., on day 0), mice were challenged with lethal dose of healthy glioblastoma stem cells (1×10⁵ cells/mouse) in the brain and monitored animals for survival. As shown in FIG. 2B, a Kaplan-Meier survival curve, the mice receiving the vaccine were 100% protected from developing the glioblastoma and survived long-term compared to 0% protection in mice receiving saline injection.

### Example 2. The Vaccine Provides Reproducible Results, Again Provided a 100% Protection from Development of the Cancer in the Mouse Model

One of skill will appreciate having some measure of the reproducibility of the results to show the reliability of the vaccine.

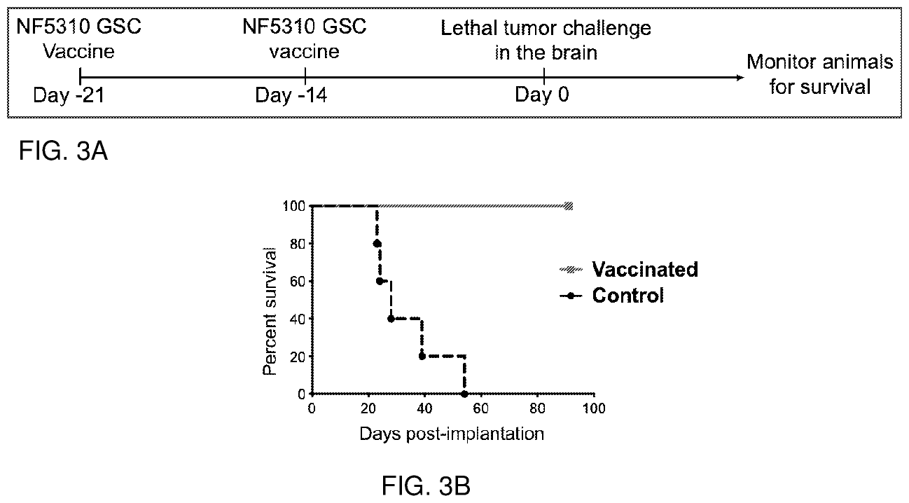

FIGS. 3A and 3B is a repeat study that verifies that the 100% protection shown in the model is reproducible, according to some embodiments. This repeated study again showed that the vaccine provided 100% protection from glioblastoma in the second immunocompetent orthotopic model (the NF5310 GBM).

FIG. 3A illustrates the treatment schedule, where the mice where again treated using the same process steps as the prior example. FIG. 3B illustrates a Kaplan-Meier survival curve. As shown in FIG. 3B, the mice receiving the vaccine were again 100% protected from developing the glioblastoma and survived long-term compared to 0% protection in mice receiving saline injection.

### Example 3. Comparing the Superiority of Stem Cells Over Tumor Cells, where the Use of a Vaccine Having Cancer Stem Cells (Glioblastoma-Specific Stem Cells) Provides Surprisingly Superior Results Over a Tumor Cell-Derived Vaccine in a Tumor Cell Derived Glioblastoma Model

One of skill will appreciate the showing of surprising results. The cancer stem cell vaccine, surprisingly, had 5× the efficacy of a tumor-cell-derived vaccine used for comparison.

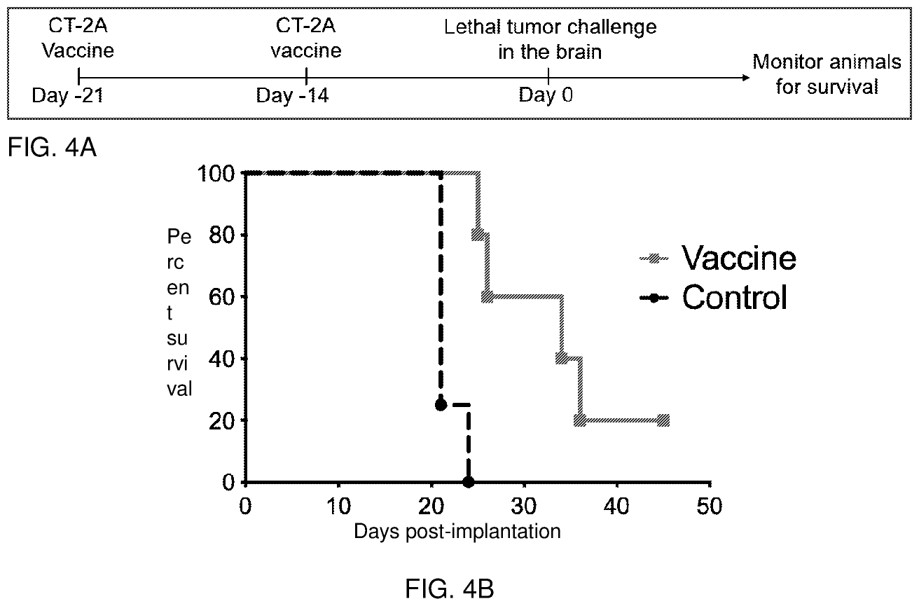

FIGS. 4A and 4B show that superiority of the cancer-stem-cell vaccine over a tumor-cell-derived vaccine, according to some embodiments. It was observed that vaccination of mice using irradiated tumor cells, as compared to cancer stem cells, provided only 20% of the protection in an orthotopic immunocompetent tumor cell-derived glioblastoma model (the CT-2A model) in C57BL/6 mice. FIG. 4A illustrates the treatment schedule. In this example, the mice were treated using mouse CT-2A tumor cells irradiated with the same 35 Gy dose radiation as used in the prior examples to induce apoptosis and create dying cancer stem cells. The dying cancer stem cells were then used as anticancer tumor cell vaccine. Apoptosis was induced in the CT-2A tumor cells by irradiating them with a 35 Gy dose of radiation to create dying cancer stem cells. The dying CT-2A tumor cells were used as the antigens to create a vaccine that was administered to the mice in the same manner as in the prior examples. The C57BL/6 mice were injected subcutaneously in the flank with irradiated CT-2A tumor cells (1×10⁶ cells/mouse), or saline as a control, on day −21 and −14. Seven days after the 2nd dose of vaccination, or saline injection, (i.e., on day 0), the mice were challenged with lethal dose of CT-2A tumor cells (1×10⁵ cells/mouse) in the brain and monitored for survival. FIG. 4B illustrates a Kaplan-Meier survival curve. As shown in FIG. 4B, the live irradiated CT-2A tumor cell vaccine provided only 1/5 of the long-term survivors when compared to the cancer stem cell vaccine. As in the earlier examples, there was 100% protection in mice receiving the vaccine, and 0% protection in mice receiving saline injection.

As such, one of skill will readily appreciate that the cancer-stem-cell vaccine provided 5× the protection of the tumor cell derived vaccine. As noted, this is a surprising result to the ordinary skilled artisan. This surprisingly superior protection obtained from the cancer stem cell vaccine is an unexpected improvement in the art of boosting anticancer immunity.

### Example 4. CD4+ T Cells are Needed for the Cancer Stem Cell Vaccine to be Effective

Interestingly, it was discovered that the efficacy of the vaccine appears to be lost in mice devoid of CD4+ T cells, not CD8+ T cells. Apparently, the efficacy of the cancer stem cell vaccine relies on the presence of CD4⁺ T cells, as demonstrated in an orthotopic 005 glioblastoma stem cell derived glioblastoma model in C57BL/6 mice.

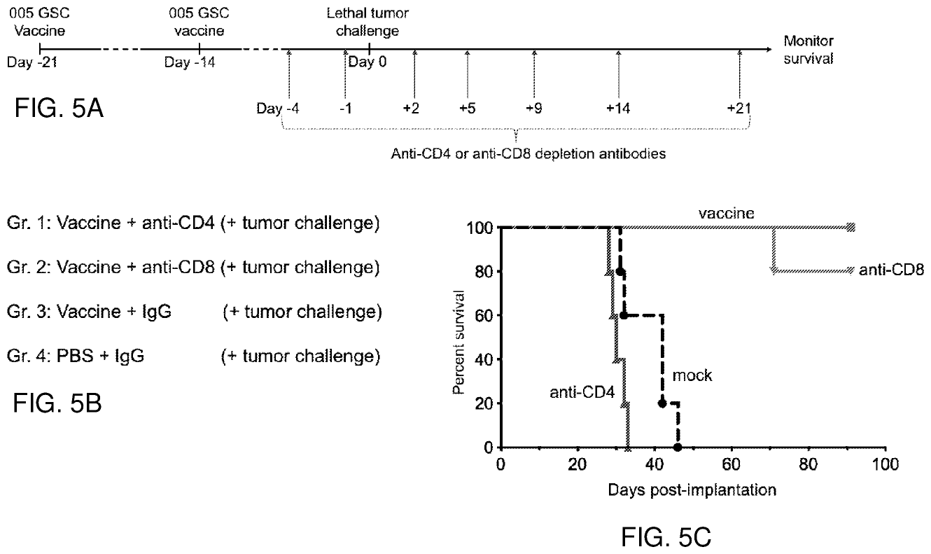

FIGS. 5A-5C show that CD4+ T cells are integral to the effectiveness of the cancer stem cell vaccine, according to some embodiments. FIG. 5A illustrates the treatment schedule. In this example, mouse 005 glioblastoma stem cells were used to prepare the cancer stem cell vaccine. The C57BL/6 mice were vaccinated on day −21 and −14, as described in the prior examples, before the mice were challenged with the healthy glioblastoma stem cells. At day 0, the mice were challenged with a lethal dose of cancer stem cells (1×10⁵ cells/mouse) in the brain and monitored animals for survival.

Concurrently, to better understand the role of immune cells (CD4⁺ and CD8⁺ T cells) in the efficacy of the vaccine, mice were injected intraperitoneally with anti-CD4, anti-CD8, or control IgG antibodies before (day −4 and −1) and after (+2, +5, +9, +14, and +21) lethal tumor challenge. FIG. 5B is a legend describing the treatment groups, and FIG. 5C is a Kaplan-Meier survival curve. FIG. 5C shows that the mice vaccinated with live irradiated cancer stem cells were again 100% protected from developing glioblastoma and survived long-term (Gr.3; line labeled “vaccine”). It also shows that the mice receiving saline had no protection and died (Gr.4; dashed line labeled “mock”). Depletion of CD8⁺ T cells did not abrogate the efficacy of the vaccine (Gr.2; 80% mice remain protected; line labeled “anti-CD8”). On the contrary, it was found that the vaccine efficacy was completely lost in the absence of CD4⁺ T cells (Gr.1; 0% protection; line labeled “anti-CD4”).

### Example 5. Cell Separation and Purity of Stem Cell Harvesting from Cancer Tissue

Cell separation was used to isolate the cancer stem cells from the cancer tissue. Any method of cell separation known to one of skill can be used. Examples of such techniques known in the art include, but are not limited to, adherence, density and antibody binding. Examples of separation types used in the art include fluorescence-activated cell sorting (FACS), magnet-activated cell sorting (MACS), pre-plating, conditioned expansion media, density gradient centrifugation, field flow fractionation (FFF), and dielectrophoresis (DEP). See, for example, Zhu, B. et al. Curr Opin Chem Eng.; 2(1): 3-7 (2013). Newer techniques also include microfluidics that use cellular properties such as elasticity in response to acoustic waves, and membrane polarization in a non-uniform electric field.

This example used an antibody-binding method of cell separation. In particular, the method of isolating cancer stem cells was as follows:
collecting surgical specimens from patients;
mechanically mincing tumor tissues;
enzymatically digesting with 0.1% Trypsin and 10 U/mL of DNasel at 37° C. for 45 min.;
after washes, triturating and passing tissues through a 100-μm cell strainer;
plating cells in EF medium composed of Neurobasal medium (Invitrogen) supplemented with 3 mmoVL L-Glutamine (Mediatech), 1× B27 supplement (Life Technologies), 0.5×N2 supplement (Life Technologies), 2 μg/mL heparin (Sigma), 20 ng/mL recombinant human EGF (R & D systems), 20 ng/mL recombinant human FGF2 (Peprotech), and 0.5× penicillin G/streptomycin sulfate/amphotericin B complex (Mediatech);
feeding the cultures for CSCs every third day with 1/3 volume of fresh medium; and,
passaging of the cultures by dissociating neurospheres using NeuroCult Chemical Dissociation kit (StemCell Technologies)
See, for example, Wakimoto, H. et al. Cancer Res. 69(8): 3472-3481 (Apr. 15, 2009); doi: 10.1158/0008-5472.CAN-08-3886 (downloaded Sep. 2, 2022), incorporated herein by reference in its entirety.

The glioblastoma-specific cancer stem cells were isolated from a glioblastoma cancer tissue sample taken from a subject. Since it is known that the cancer stem cells express the CD133 stem cell marker, the cells in the cancer tissue sample were labeled with CD133 antibody for to identify the cancer stem cells and separate them from the cancer tissue sample. IgG was the control antibody used.

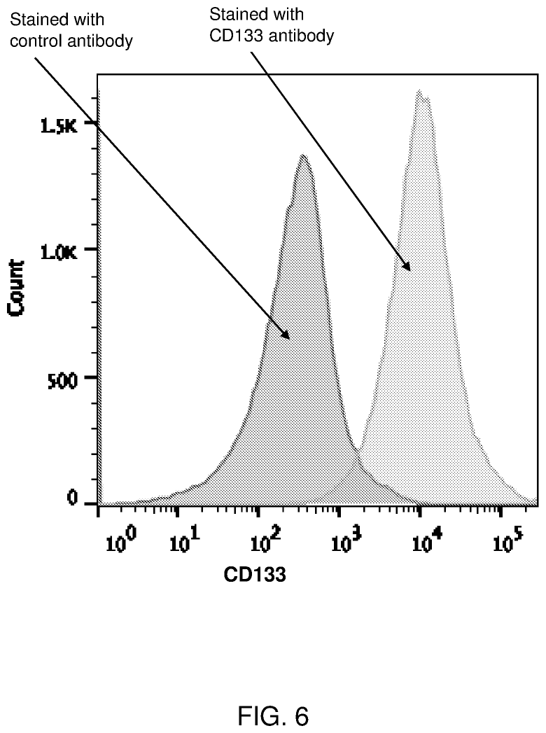

FIG. 6 shows the stemness of the pure, cancer cancer stem cells, according to some embodiments. Glioblastoma stem cells were isolated from a cancer tissue sample. The isolated, pure cancer stem cells were treated with 35 Gy radiation at a dose rate of 0.740 Gy/min for about 47 minutes to create dying cancer stem cells. The dying cancer stem cells were then injected into the mice immediately as a vaccine, died in the mice after a period of 10-14 days, and the dead cancer stem cells were then phagocytosed by macrophages and/or dendritic cells in the mice to induce antitumor immunity.

### Example 6. Brain Tissue Samples Verify that the Vaccine Kills Glioblastoma Cancer Stem Cells in the Brain

This example provides direct evidence that the vaccine kills developing glioblastoma cancer tissue in the brains of the mice receiving the vaccine.

Glioblastoma stem cells were exposed to 35 Gy radiation to create dying stem cells, and the C57BL/6 mice were vaccinated with the dying glioblastoma stem cells at days −21 and −14. At day 0, the mice were challenged with healthy glioblastoma stell cells. The brain tissue from the mice was collected at day 21 and tested for glioblastoma cells.

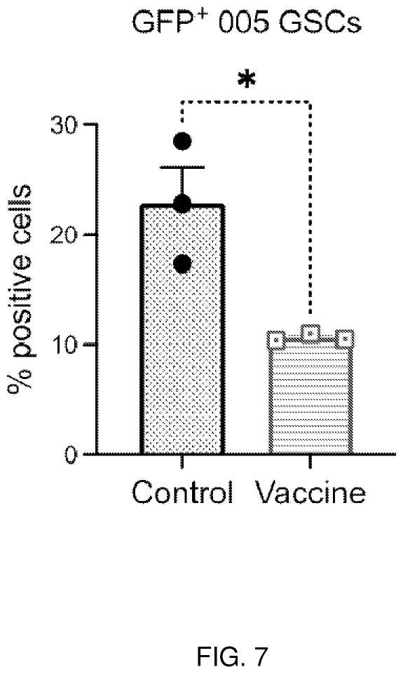

FIG. 7 shows an analysis of brain tissue, namely that mice vaccinated with the cancer stem cell vaccine had a significantly smaller number of cancer stem cells in the brain tissue than mice that did not receive the vaccine, according to some embodiments. As illustrated in FIG. 7, the mice vaccinated with the glioblastoma-specific stem cell vaccine had a significantly smaller number of glioblastoma stem cells in the brain tissue than mice that did not receive the vaccine. This example provides proof that the vaccine reaches and kills glioblastoma cancer stem cells in the brain.

### Example 7. Eradicating Established Tumors with the Vaccine

The above data provides sufficient evidence for one of skill in the art to reasonably believe that the vaccine will eradicated established glioblastoma tumors. This example will be used to obtain data that shows that a post-challenge vaccination will eradicate established glioblastoma tumors.

Mice will be first challenged with healthy glioblastoma stem cells that will give rise to a tumor in the brain. The vaccine, created as taught herein, will be administered post-challenge on day 7, 14, and 21; or, perhaps day 3, 10, and 17. As in the earlier examples, some mice will receive the vaccine, and some mice will receive the control, saline solution only.

It is expected that the results will show 100% eradication of the glioblastoma in mice receiving the vaccine, and 0% eradication of the glioblastoma in mice receiving saline injection.

### Example 8. Using Reprogrammed Cancer Stem Cells as a Delivery Vehicle for a “Triple-Killer” Cancer Treatment

The methods can include using reprogrammed cancer stem cells as a triple-killer cancer treatment. The treatment administers live, cancer stem cells that are re-programmed with an oncolytic virus that expresses an immunomodulatory cytokine, wherein the cancer treatment (1) kills the cancer stem cells after administration in a subject to release a cancer antigen cocktail that creates cancer antibodies in the subject, (2) kills cancer tumor cells in the subject through release of the oncolytic virus itself, and (3) kills additional cancer tumor cells in the subject, the immunomodulatory cytokine further providing an immunological memory response. Moreover, the oncolytic virus is amplified in the cancer stem cell itself, avoiding the toxicity associated with a pure administration of the immunomodulatory cytokine. Less oncolytic virus can be administered when administered through cancer stem cells when compared to I.V. or I.M. administrations of the oncolytic virus itself. Likewise, less of the toxic immunomodulatory cytokine is administered through cancer stem cells when compared to I.V. or I.M. administrations of the cytokine itself. The amount of cells administered can be the same as taught in this application for administration of the cancer stem cells as a vaccine.

Generally speaking, the method includes constructing and amplifying a cancer oncolytic virus that expresses immunomodulatory cytokines. Cytokines are low molecular weight, soluble proteins that are produced in response to an antigen and function as chemical messengers for regulating the innate and adaptive immune systems. As such, cytokines can be effective in the treatment of cancers.

In some embodiments, the immunomodulatory cytokines include IFN-α and IL-2, and their recombinant forms and derivatives thereof. And, in some embodiments, the immunomodulatory cytokines include IL-12, IL-15, IL-18 and IL-21 and their recombinant forms and derivatives thereof. The following table lists these cytokines, their target cells and receptor expression, immunomodulatory effects, their amino acid sequences, and the sequences of the genes that express them. Their recombinant forms can be used in some embodiments.

TABLE 3

| | Target cells/ | | Amino acid | Nucleotide |
|---|---|---|---|---|
| Cytokine | receptor expression | Immunomodulatory effects | SEQ ID NO: | SEQ ID NO: |
| IFN-α | Most somatic cells | Promotes proliferation and activation of NK and cytotoxic T cells | 1-2, 29-30 | 3-4, 31-32 |
| IL-2 | T lymphocytes, NK cells | Promotes proliferation and activation of cytotoxic T cells; survival | 5-6, 41-42 | 7-8, 43-44 |
| | | of Tregs | | |
| IL-12 | T lymphocytes, NK cells | Promotes proliferation and activation of NK and cytotoxic T cells; | 9-12 | 13-16 |
| | | secretion of IFN-γ | | |
| IL-15 | Cytotoxic and memory T | Promotes survival of CD4+, CD8+ and memory T lymphocytes and NK | 17-18 | 19-20 |
| | lymphocytes, NK cells | cells | | |
| IL-18 | B, T lymph; NK cells, | Promotes proliferation and activation of NK and cytotoxic T cells; | 21-22, 53 | 23-24, 54 |
| | Mϕ, fibroblasts, | secretion of IFN-γ; inhibits proliferation of Tregs | | |
| | melanocytes, endothelial, | | | |
| | epithelial cells | | | |
| IL-21 | NK, NKT, DC, Mϕ, B | promotes proliferation of activated B cells, activated NK and NKT cells, | 25-26, 55 | 27-28, 56 |
| | lymphocytes, T helper | and cytotoxic T lymphocytes, and differentiation of plasma cells | | |
| | and cytotoxic T lymphocytes | | | |

DC: Dendritic cell;
Mϕ: Macrophage

In fact, IFN-α, IL-12, IL-18, and IL-21 have been tested in Phase I and II trials, at least to the extent of direct administration by intravenous (I.V.) and subcutaneous (sc.) routes. In some embodiments, IFN-α and it's recombinant forms and derivatives thereof can be used in the treatment of cancers, including metastatic cancers, such as melanoma and malignant melanoma. There are 13 different IFN-α genes in humans, and each can be used in an embodiment herein: IFN-α1, IFN-α2, IFN-α4, IFN-α5, IFN-α6, IFN-α7, IFN-α8, IFN-α10, IFN-α13, IFN-α14, IFN-α16, IFN-α17, and IFN-α21. In some embodiments, the IFN-α is INTRON A (Interferon alpha-2b). As such, the nucleotide sequence encoding IFN alpha-2b, can be inserted into an oncolytic virus taught herein. In some embodiments, the IFN-α is ROFERON-A (Interferon alpha-2a, recombinant). As such, the nucleotide sequence encoding IFN alpha-2a, can be inserted into an oncolytic virus taught herein.

In some embodiments, an IFN-α can be administered in doses ranging from 3 MU/m² to 100 MU/m², or any range therein in increments of 0.5 mU/m². In some embodiments, the amount of IFN-alpha administered can be set at a delivery equivalent to 20 million IU/m² intravenously five times per week for 4 weeks (induction phase) followed by 10 million IU/m² subcutaneously three times per week for 48 weeks (maintenance phase). In these embodiments, the IFN-alpha can be INTRON A.

In some embodiments, IFN-γ and its recombinant forms and derivatives thereof, can be used in the treatment of cancers, including lung cancer, including non-small cell lung cancer, colon cancer, and melanoma as it has a pro-apoptotic effect on cancer cells. In some embodiments, the IFN-γ is human (h IFN-γ) (SEQ ID NO. 33). As such, the nucleotide sequence encoding hIFN-γ (SEQ ID NO: 35), can be inserted into an oncolytic virus taught herein. In some embodiments, the IFN-γ is mouse (mIFN-γ) (SEQ ID NO. 34). As such, the nucleotide sequence encoding mIFN-γ (SEQ ID NO: 36), can be inserted into an oncolytic virus taught herein.

An IFN-γ can also inhibit angiogenesis, which has an anti-tumor effect. In some embodiments, IFN-γ can be administered in combination with tumor necrosis factor-alpha (TNF-α), such as human (hTNF-α) (SEQ ID NO. 37) and mouse (mTNF-α) (SEQ ID NO. 38). As such, the gene encoding TNF-α can be inserted into an oncolytic virus taught herein, such as human (hTNF-α) (SEQ ID NO. 39) and mouse (mTNF-α) (SEQ ID NO. 40), either alone or in combination with an IFN-γ. In some embodiments, an IFN-γ can be administered in the range of 10 ug/m² to 300 ug/m², 20 ug/m² to 200 ug/m², 30 ug/m² to 300 ug/m², 40 ug/m² to 400 ug/m², 50 ug/m² to 200 ug/m², or any amount or range therein in increments 5 ug/m². TNF-α can be administered in the range of 10 ug/m² to 200 ug/m², 20 ug/m² to 200 ug/m², 30 ug/m² to 100 ug/m², 40 ug/m² to 80 ug/m², 50 ug/m² to 75 ug/m², or any amount or range therein in increments 5 ug/m². As such, combinations of IFN-γ and TNF-α can be administered in any combination of those amounts. For example, in some embodiments, a combination of IFN-γ and TNF-α can be administered using 100 ug/m² IFN-γ and 50 ug/m² TNF-α.

In some embodiments, IL-2, and its recombinant forms and derivatives thereof, can be administered, which can be selected from human (hIL-2) (SEQ ID NO: 5), mouse (mIL-2) (SEQ ID NO: 6), recombinant human IL-2 (rhIL-2; aldesleukin) (SEQ ID NO: 41), recombinant mouse IL-2 (rmIL-2; aldesleukin) (SEQ ID NO: 42), or any combination thereof, for treatment of any cancer taught herein. The cancers can include, for example, melanoma, and malignant melanoma, in some embodiments. As such, the nucleotide sequence encoding human (hIL-2) (SEQ ID NO: 7), mouse (mIL-2) (SEQ ID NO: 8), recombinant human IL-2 (rhIL-2; aldesleukin) (SEQ ID NO: 43), recombinant mouse IL-2 (rmIL-2; aldesleukin) (SEQ ID NO: 44), or any combination thereof, can be inserted into an oncolytic virus taught herein. In some embodiments, rhIL-2 can be administered in doses ranging from 100,000 IU/kg to 1,000,000 IU/kg, or any range therein in increments of 1000 IU/kg. In some embodiments, rhIL-2 can be administered in doses ranging from 500,000 IU/kg to 750,000 IU/kg, or any range therein in increments of 1000 IU/kg. In some embodiments, rhIL-2 can be administered in doses ranging from 600,000 IU/kg to 720,000 IU/kg, or any range therein in increments of 1000 IU/kg. In some embodiments, the rhIL-2 can be administered in intervals, for example, 2 week, 4 week, 8 week, or 12 week intervals for 28 week periods, 1 year periods, and the like. In some embodiments, IL-2 can be administered in combination with GM-CSF whether human (SEQ ID NO: 45) or mouse (SEQ ID NO: 46). The combination can be administered, for example, following lympho-depleting chemotherapy with cyclophosphamide. As such, the nucleotide sequence encoding GM-CSF, whether human (SEQ ID NO: 47) or mouse (SEQ ID NO: 48), can be inserted into an oncolytic virus taught herein. In some embodiments, any GM-CSF taught herein can be administered alone or in combination with any of the other desired proteins or peptides taught herein. In some embodiments, the desired protein is a glycoprotein from a gene encoding mouse granulocyte macrophage colony stimulating factor (mGM-CSF) (SEQ ID NO: 57), and the nucleotide sequence encoding the mouse GM-CSF glycoprotein (SEQ ID NO: 58), can be inserted into an oncolytic virus taught herein. In some embodiments, the nucleotide sequence encoding a desired mouse GM-CSF protein is codon optimized (SEQ ID NO: 59). In some embodiments, the desired protein is a glycoprotein from a gene encoding human granulocyte macrophage colony stimulating factor (hGM-CSF) (SEQ ID NO: 60), and the nucleotide sequence encoding the human GM-CSF glycoprotein (SEQ ID NO: 61), can be inserted into an oncolytic virus taught herein. In some embodiments, the nucleotide sequence encoding a desired human GM-CSF protein is codon optimized (SEQ ID NO: 62).

In some embodiments, IL-12, and it's recombinant forms and derivatives thereof, can be used in the treatment of cancers, for example, renal cancer, melanoma, and colon cancer. An example of an IL-12 that can be used includes, but is not limited to, recombinant human interleukin-12 (rhIL-12), available from Hoffman-LaRoche. As such, the nucleotide sequence encoding rhIL-12 can be inserted into an oncolytic virus taught herein.

In some embodiments, the IL-12 is a subunit, such as a p35 subunit or p40 subunit. The p35 subunit of the IL-12 can be human (SEQ ID NO: 9) or mouse (SEQ ID NO: 11). As such, the nucleotide sequence encoding the p35 subunit of the IL-12 can be human (SEQ ID NO: 13) or mouse (SEQ ID NO: 15) can be inserted into an oncolytic virus taught herein. The p40 subunit of the IL-12 can be human (SEQ ID NO: 10) or mouse (SEQ ID NO: 12). As such, the nucleotide sequence encoding the p40 subunit of the IL-12 can be human (SEQ ID NO: 14) or mouse (SEQ ID NO: 16) can be inserted into an oncolytic virus taught herein.

In some embodiments, an IL-12, such as the rhIL-12 for example, can be administered in combination with interferon-α2b (IFN-α2b), available from InvivoGen, San Diego, CA. As such, the nucleotide sequence encoding IFN-α2b, can be inserted into an oncolytic virus taught herein.

In some embodiments, an IL-12 can be administered in combination with human Melan-A peptide (SEQ ID NO: 49) which includes residues 26-35 EAAGIGILTV (SEQ ID NO: 127). As such, the nucleotide sequence encoding the human Melan-A peptide (SEQ ID NO: 51) which includes residues 26-35 EAAGIGILTV (SEQ ID NO: 127), can be inserted into an oncolytic virus taught herein. In some embodiments, an IL-12 can be administered in combination with mouse Melan-A peptide (SEQ ID NO: 50) which includes residues 24-32 EAAGIGILIV (SEQ ID NO: 128). As such, the nucleotide sequence encoding the mouse Melan-A peptide (SEQ ID NO: 52) which includes residues 24-32 EAAGIGILIV (SEQ ID NO: 128), can be inserted into an oncolytic virus taught herein.

In some embodiments, an IL-12 can be administered in doses selected from the group consisting of 10 ng/kg, 30 ng/kg, 100 ng/kg, 500 ng/kg, and any amount or range therein. In some embodiments, the IL-12 can be administered in doses ranging from 10 ng/kg to 500 ng/kg, from 10 ng/kg to 100 ng/kg, from 30 ng/kg to 100 ng/kg, from 10 ng/kg to 30 ng/kg, or any amount or range therein in increments of 1 ng/kg. In some embodiments, the human or mouse Melan-A peptide can be administered in an amount ranging from 0.005 nM to 0.500 nM, from 0.0075 nM to 0.250 nM, from 0.010 nM to 0.100 nM, or any amount or range therein in increments of 0.001 nM.

In some embodiments, IL-15, and it's recombinant forms and derivatives thereof, can be used in the treatment of cancers, for example, melanoma, and malignant melanoma. In some embodiments, the IL-15 can be IL-15 isoform 1. As such, the nucleotide sequence encoding IL-15 isoform 1 can be inserted into an oncolytic virus taught herein. In some embodiments, the IL-15 can be IL-15 isoform 2. As such, the nucleotide sequence encoding IL-15 isoform 2, can be inserted into an oncolytic virus taught herein.

In some embodiments, the IL-15 can be human (SEQ ID NO: 17) or mouse (SEQ ID NO: 18). As such, the nucleotide sequence encoding the human IL-15 (SEQ ID NO: 19) or the mouse IL-15 (SEQ ID NO: 20) can be inserted into an oncolytic virus taught herein.

In some embodiments, IL-15 can be administered in doses ranging from 0.1 ug/kg/day to 100 ug/kg/day, 0.05 ug/kg/day to 50 ug/kg/day, 0.3 ug/kg/day to 25 ug/kg/day, or any amount or range therein in increments of 0.1 ug/kg/day. Various forms of IL-15 and recombinant IL-15 are available from PeproTech, Cranbury, NJ.

In some embodiments, IL-18, and it's recombinant forms and derivatives thereof, can be used in the treatment of cancers, for example, melanoma, malignant melanoma, renal cancer, lymphoma, ovarian cancer, breast cancer, and testicular and prostatic cancer. An example of an IL-18 that can be used is Iboctadekin/SB-485232, a recombinant human IL-18, rhIL-18, available from GlaxoSmithKline (SEQ ID NO: 53). As such, the nucleotide sequence encoding IL-18 (SEQ ID NO: 54), can be inserted into an oncolytic virus taught herein.

In some embodiments, the IL-18 can be human (SEQ ID NO: 21) or mouse (SEQ ID NO: 22). As such, the nucleotide sequence encoding the human IL-15 (SEQ ID NO: 23) or the mouse IL-15 (SEQ ID NO: 24) can be inserted into an oncolytic virus taught herein.

In some embodiments, IL-18 can be administered in combination with doxorubicin (Doxil), such as pegylated liposomal doxorubicin, also available from GlaxoSmithKline, particularly in patients with ovarian, breast, testicular, and prostatic cancer. The IL-18 and rhIL-18 can be administered in doses ranging from 50 ug/kg to 3000 ug/kg, from 100 ug/kg to 2000 ug/kg, from 500 ug/kg to 2000 ug/kg, from 500 ug/kg to 1000 ug/kg, from 1000 ug/kg to 200 ug/kg, or any amount or range therein in increments of 10 ug/kg. As with any of the cytokine administrations taught herein, the administration can be cycled in daily, multi-daily, weekly, multi-weekly, or monthly increments.

In some embodiments, IL-21 can be used in the treatment of cancers, for example, melanoma and malignant melanoma. In some embodiments, the IL-21 is a recombinant human IL-21 (rhIL-21; denenicokin) available from Novo Nordisk/ZymoGenetics (SEQ ID NO: 55). As such, the nucleotide sequence encoding rhIL-21, denenicokin, (SEQ ID NO: 56), can be inserted into an oncolytic virus taught herein. In some embodiments, the IL-21 can be human (SEQ ID NO: 25) or mouse (SEQ ID NO: 26). As such, the nucleotide sequence encoding the human IL-15 (SEQ ID NO: 27) or the mouse IL-15 (SEQ ID NO: 28) can be inserted into an oncolytic virus taught herein.

In some embodiments, the IL-21 and rhIL-21 can be administered in doses ranging from 5 ug/kg to 500 ug/kg, from 20 ug/kg to 200 ug/kg, from 30 ug/kg to 300 ug/kg, or any amount or range therein in increments of 1 ug/kg.

### Manufacturing a Medicament

The delivery systems taught herein can be referred to as a medicament in some embodiments. One of skill will appreciate that a method of manufacturing a medicament is provided. In some embodiments, the medicament is a vaccine, and the method includes obtaining the cancer stem cells and programming the cancer stem cells to die after administration of the medicament to a subject, the programming of the cancer stem cells done according to the teachings provided herein. In some embodiments, the method also includes loading the cancer stem cells with an oncolytic virus, in addition to programming the cancer stem cells to die in a desired time after administration of the medicament to a subject. In some embodiments, before loading the cancer stem cells with the oncolytic virus, the oncolytic virus is further engineered to express a desired protein.

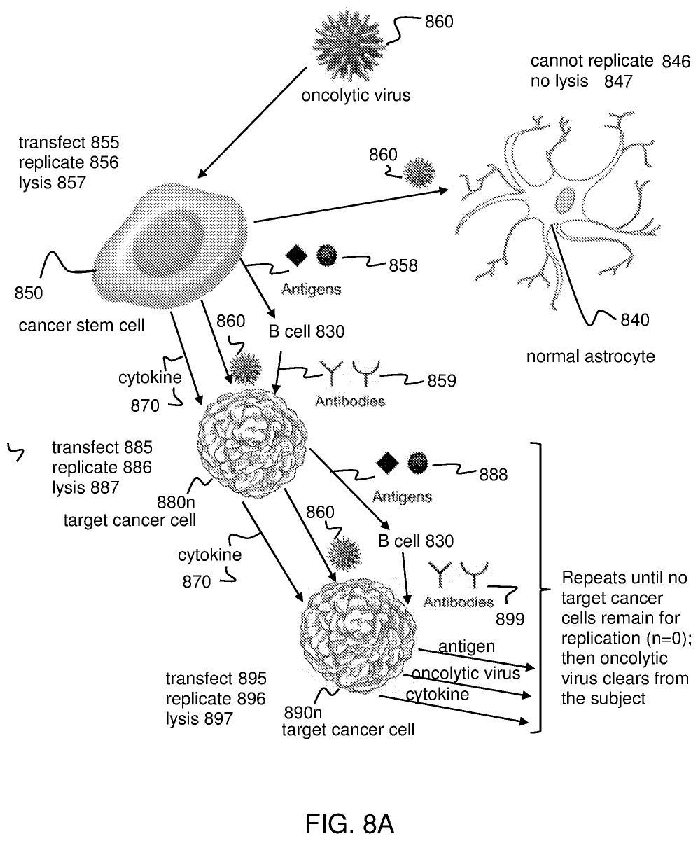

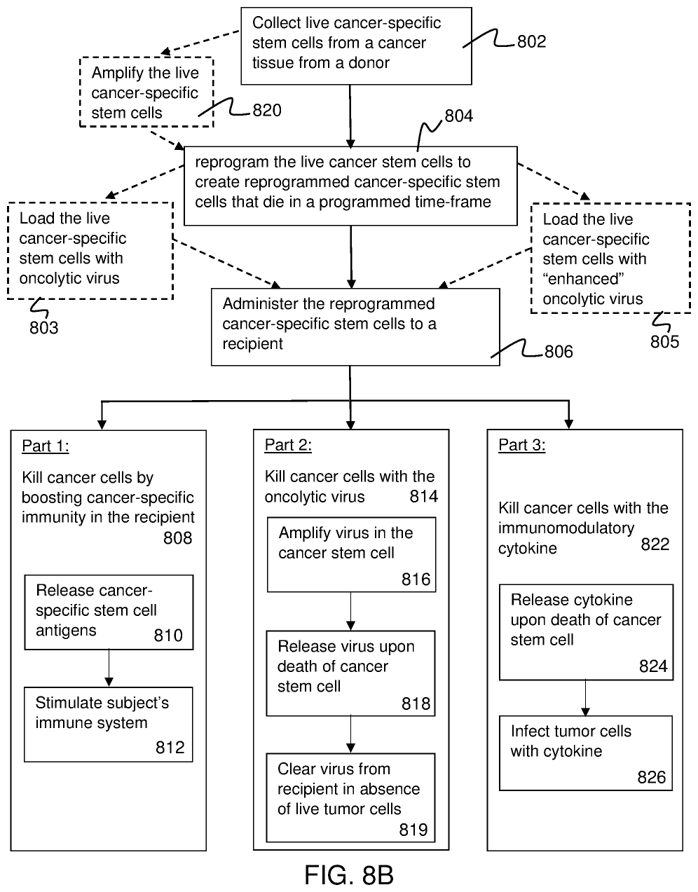

FIGS. 8A and 8B illustrate transfection of a cancer stem cell by a tumor-specific oncolytic virus leading to the triple-killer properties, and a flowchart showing a general method of using cancer stem cells as a triple-killer cancer treatment, according to some embodiments.

FIG. 8A illustrates the process of transfecting a cancer stem cell 850 with a tumor-specific oncolytic virus 860 in a subject. The oncolytic virus 860 is “tumor specific”, meaning that it is engineered to replicate 856 preferentially, in the cancer stem cell 850 and the target cancer cell 880n. In some embodiments, the oncolytic virus 860 replicates only in the cancer stem cell 850 and the target cancer cell 880n. A normal astrocyte 840 is not affected by the virus 860, because the oncolytic virus is tumor-specific and cannot transfect and replicate in the normal astrocyte 840. As such, since there is no replication 846 of the oncolytic virus 860 in, or lysis of, the normal astrocyte 840, this copy of the tumor-specific oncolytic virus 860 will either die or find a respective cancer in which to replicate and live. In the absence of such cancer cells, all copies of the oncolytic virus will die and leave the subject. The oncolytic virus 860 can express a cytokine 870, in some embodiments. Consistent with the teachings presented earlier in this document, the cancer stem cells 850 are programmed to die through the transfection of the cancer stem cells 850 with the oncolytic virus 860. The programming can include, for example, any treatment that results of the death of the cancer stem cells at a desired time following administration of the cancer stem cells to a subject. In some embodiments, radiation treatment of the cancer stem cell 850 can be used alone, or in some embodiments, in combination with the transfection 855 with the oncolytic virus 860, in programming the timely death of the cancer stem cell 850 to release antigen, virus. In some embodiments, the oncolytic virus expresses a desired protein, such as a desired cytokine in the treatment.

The transfection of the cancer stem cell 850 with the oncolytic virus 860 provides the dual-killer and triple-killer effects. The death of the cancer stem cell results in a lysis 857 of the cancer stem cell 850 and release of cancer stem cell antigens 858 in the production of antibodies 859 by B cells 830, which is used by the adaptive immune system to boost cancer immunity in the subject and provide a first mechanism to kill target cancer cells 880n in the triple-killer process, where n is the total amount of target cancer cells in the subject. The oncolytic virus 860 is also released upon the lysis 857 of the cancer stem cell and, since the oncolytic virus 860 itself can kill the target cancer cells 880n, this provides a second mechanism to kill target cancer cells in the triple-killer process. Finally, since the oncolytic virus 860 can express cytokine 870 in the cancer stem cell 850, the lysis 857 of the cancer stem cell 850 will result in the release of cytokine 870 which provides a third mechanism to kill target cancer cells 880n in the triple-killer process.

The process of transfection 885, replication 886, lysis 887, and release of antigens 888, oncolytic virus 860, and cytokine 870 repeats in the target cancer cells 880n, and the process moves on to target cancer cells 890n, and so on, repeating the transfection 895, replication 896, and lysis 897, etc., until there are no target cancer cells remaining for replication of the “tumor specific” oncolytic virus in the subject, n=0, after which the oncolytic virus 860 clears from the subject.

FIG. 8B shows steps in a method used to at least inhibit, prevent, or treat a cancer in a subject. The treatment could be a prophylactic treatment to at least inhibit, and possibly prevent, the development of cancer cells in a subject that may, for example, have a genetic predisposition for the cancer. In some embodiments, a treatment provided herein could be used to kill small amounts of undetected new cancer cells, or re-emergent cancer cells in a complete or partial remission. The methods can include, for example, collecting 802 live, cancer stem cells from a cancer tissue from a donor, and reprogramming 804 the live cancer stem cells to create reprogrammed cancer stem cells that die in a programmed time-frame for use as the active components in an anti-cancer vaccine. Likewise, the cancer stem cells can be loaded 803 with a tumor-selective, oncolytic virus, in some embodiments, the oncolytic virus configured for replicating in only the tumor cells and, thus, dying in the absence of tumor cells. Moreover, the cancer stem cells can be loaded 805 with an enhanced, tumor-selective, oncolytic virus which is also configured for replicating in only the tumor cells and, thus, dying in the absence of tumor cells.

The method includes the administering 806 of the reprogrammed cancer stem cells to a recipient, the dying of the cancer stem cells occurring after the reprogramming and administering of the cancer stem cells to the recipient to treat, prevent, or at least inhibit a development of, the cancer in the recipient. The administering 806 leads to part 1 of the triple-killer cancer treatment: a killing of cancer tumor cells by a boosting 808 of a cancer immunity in the recipient, the boosting 808 including releasing 810 cancer stem cell antigens, and stimulating 812 the recipient's immune system; wherein, the releasing and stimulating results in boosting the cancer immunity against the solid cancer. Part 2 of the triple-killer cancer treatment is a killing 814 of cancer tumor cells with the oncolytic virus; wherein, the killing 814 includes amplifying 816 the virus in the cancer stem cells, releasing 818 the virus upon a death of the cancer stem cells, and clearing 819 the virus from the recipient of the cancer stem cells in the absence of live cancer tumor cells. Part 3 of the triple-killer cancer treatment is a killing 822 of the cancer tumor cells with the immunomodulatory cytokine that is expressed by the oncolytic virus; wherein, the killing 822 includes releasing 824 the cytokine upon the death of the cancer stem cell, and infecting 826 cancer tumor cells with the cytokine.

In the methods taught herein, a cancer oncolytic virus is constructed and/or amplified to express an immunomodulatory cytokine such as, for example, a cytokine selected from the group consisting of IFN-α, IL-2, IL-12, IL-15, IL-18, and IL-21. And, cancer stem cells are collected from a donor and amplified, where the donor can be the recipient.

The cancer stem cells are infected with the oncolytic virus that expresses the immunomodulatory cytokine, where the oncolytic virus kills the cancer stem cell in a desired time-frame after the administration of the live, cancer stem cells. The time at which the death of the cancer stem cells occurs can be controlled. In some embodiments, the cancer stem cell can be pretreated either before, or after, infection with the virus to vary the time to apoptosis. In some embodiments, the cancer stem cell can be pretreated before and after infection with the virus to vary the time to apoptosis. A pretreatment may include radiation treatment, in some embodiments.

In some embodiments, due to the programming, the cancer stem cells die in 1 day, 2 days, 3 days, 4 days, 5 days, 6 days, 7 days, 8 days, 9 days, 10 days, 11 days, 12 days, 13 days, 14 days, or any amount or range of time therein in increments of 4 hours. In some embodiments, the cancer stem cells die in a range of 1 day to 14 days, 2 days to 10 days, 3 days to 7 days, 4 days to 6 days, 10 days to 12 days, or any amount or range therein in increments of 4 hours.

In some embodiments, the oncolytic virus kills the cancer stem cells in 3 days. In some embodiments the oncolytic virus kills the cancer stem cells in 4 days. In some embodiments, the oncolytic virus kills the cancer stem cells in 5 days. In some embodiments the oncolytic virus kills the cancer stem cells in 6 days. In some embodiments, the oncolytic virus kills the cancer stem cells in 7 days. In some embodiments the oncolytic virus kills the cancer stem cells in 8 days. In some embodiments the oncolytic virus kills the cancer stem cells in 9 days. In some embodiments the oncolytic virus kills the cancer stem cells in 10 days. In some embodiments the oncolytic virus kills the cancer stem cells in 11 days. In some embodiments the oncolytic virus kills the cancer stem cells in 12 days. In some embodiments the oncolytic virus kills the cancer stem cells in 13 days. In some embodiments the oncolytic virus kills the cancer stem cells in 14 days.

The live, reprogrammed cancer stem cells are administered to a subject, the cancer stem cells die in a desired time frame and release the oncolytic virus. The oncolytic virus and the expressed cytokine the kill cancer tumor cells. Once the cancer tumor cells are gone, the oncolytic virus is then vulnerable to the subject's immune system, such that the configuration of the oncolytic virus helps to ensure that it will clear from the subject.

As noted above, the dead cancer stem cells release many potential cancer related antigens (more antigens than the mature tumor cells, with potential antigens to fight against cancer resistance) which can stimulate the systematic immune responses to detect and kill most types of tumor cells, whether local or distant, including glioblastoma tumors as shown herein.

The oncolytic viruses kill tumor cells by oncolysis, but they can also activate anti-tumor immunity in the subject treated by themselves. During the virus amplification in the preexisting tumor cells, the oncolytic viruses express and release the immunomodulatory cytokines and activate the anti-tumor immunity.

When there are no more tumor cells in which the oncolytic virus can replicate, an immunological memory that was developed by the treatment will protect the subject from future occurring tumors. The oncolytic viruses will be slowly eliminated from the body without attacking the healthy cells or starting the expression of immune stimulating genes. It will only take three to seven days to eliminate the viruses without the existing tumor cells, and so adverse reactions caused by the cytokines will be minimal.

### Example 9. Construction of an Oncolytic Virus. Expression of IL-2 from Vero Cells. Infection of Glioblastoma Stem Cells. And Viability of Glioblastoma Stem Cells with Varying Amounts of Infection

This example discusses state-of-the-art references that show the construction of an oncolytic virus that has been configured for selective replication in tumor cells, and, in some embodiments, configured to express desired proteins. See U.S. Pat. No. 10,570,377 (“Coffin”) and Thomas et al. Journal for ImmunoTherapy of Cancer 7:214 (2019) (“Thomas”), each of which is hereby incorporated herein by reference in it's entirety.

Data is provided showing the expression of IL-2, including the expression of the IL-2 from vero cells, the infection of glioblastoma stem cells, and the viability of the glioblastoma stem cells after infection with varying amounts of the oncolytic virus. The flexibility of genetic engineering methods available to the skilled artisan allows an oncolytic virus to be constructed as-desired. In addition to constructing the virus to be tumor-specific, for example, the virus can be designed to express any desired protein, mouse or human or otherwise. For example, the desired protein can be an immunomodulatory cytokine, in some embodiments.

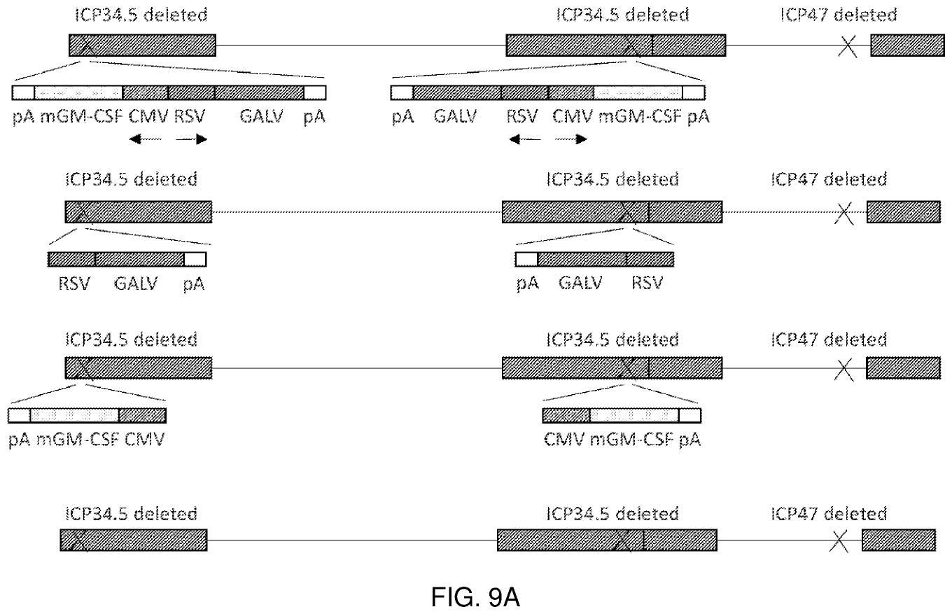

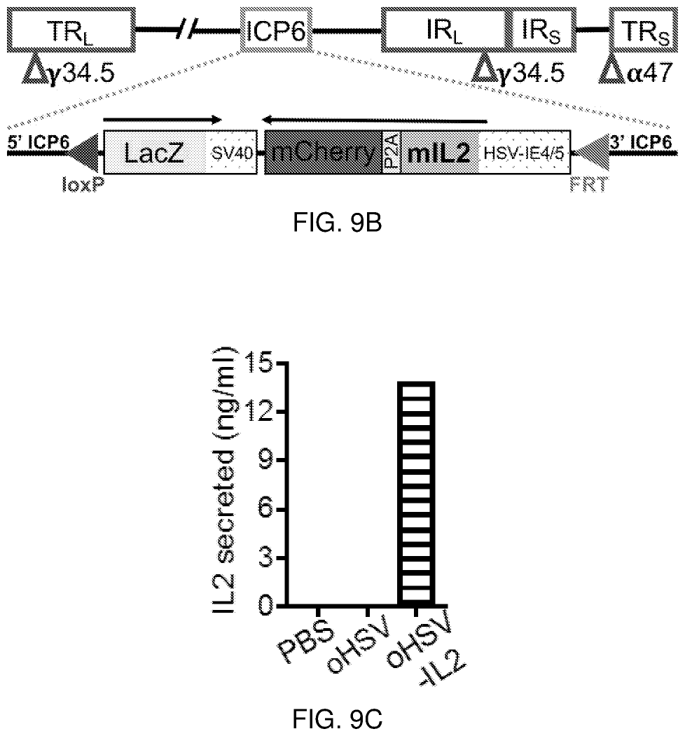

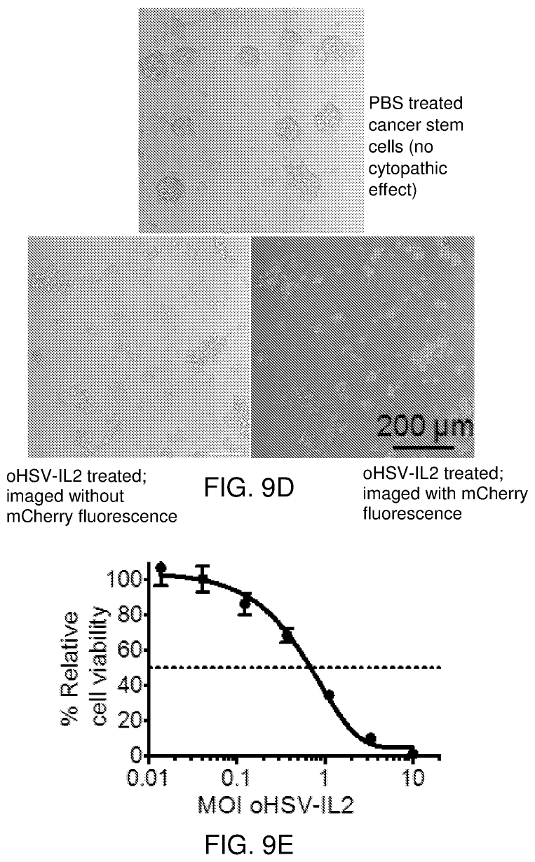

FIGS. 9A-9E illustrate the construct of an oncolytic virus expressing IL-2 and mCherry, according to some embodiments. FIG. 9A illustrates the construction of an HSV virus in which the ICP34.5 gene is deleted so the virus is tumor-specific, meaning that it can only replicate in tumor cells, and it will die when the tumor cells are no longer available to the virus; and, the ICP47 gene is deleted to enhance viral and tumor antigen presentation by major histocompatibility complex-I (MHC-1), according to some embodiments. In panel 1,
a glycoprotein from a gene encoding granulocyte macrophage colony stimulating factor (GM-CSF), for example, (mGM-CSF) (SEQ ID NO: 57), is expressed by a nucleotide sequence encoding the mGM-CSF (SEQ ID NO: 58), or an optimized nucleotide sequence thereof (SEQ ID NO: 59); or, human GM-CSF (hGM-CSF) (SEQ ID NO: 60), expressed by a nucleotide sequence encoding the hGM-CSF (SEQ ID NO: 61), or an optimized nucleotide sequence thereof (SEQ ID NO: 62); and,
gibbon ape leukemia virus protein (GALV) having the R transmembrane peptide mutated or removed (GALV-R) (SEQ ID NO: 63), expressed by a nucleotide sequence encoding the GALV-R (SEQ ID NO: 64) or an optimized version thereof (SEQ ID NO: 65); wherein, the genes are both inserted into the ICP34.5 gene locus for expression of both proteins; and, the ICP47 gene is deleted such that the US11 gene is under the control of the ICP47 immediate early promoter.

In panel 2, only a GALV-R gene is inserted so that only GALV-R is expressed. In panel 3, only an mGM-CSF gene is inserted so that mGM-CSF is expressed. In panel 4, the virus can be constructed with no gene inserted to express a desired protein or combination of proteins, such as in the case of the dual killer method of treatment that relies on the combined activities of (i) the antigens released from the dead cancer stem cells and (ii) the oncolytic virus alone, in which the virus is engineered to replicate in tumor tissue only, or at least primarily, such that the virus is all, or at least substantially, eliminated from the system in the absence of tumor tissue.

FIG. 9B illustrates the construction of an HSV oncolytic virus, according to some embodiments. Namely, FIG. 9B illustrates the configuration of the oncolytic virus oHSV-IL2 (oncolytic herpes simplex virus expressing interleukin 2), that is constructed to express (i) beta-galactosidase from a LacZ reporter gene; (ii) mCherry, a monomeric red fluorescent protein (mRFP) that absorbs light between 540-590 nm and emits light in the range of 550-650 nm, a fluorescent protein chromophore used to visualize genes and analyze their functions in experiments; and, (iii) the cytokine IL-2. “loxP” is a recognition sequence, 34-bp long, consisting of two 13-bp long palindromic repeats separated by an 8-bp long asymmetric core spacer sequence. “sv40” is Simian Virus 40 promoter; “P2A” is a self-cleaving sequence that separates the protein expressions between the mCherry protein and the miL2 protein; the “HSV-IE4/5” is a promoter that drives the expression of the DNA; “TR_{L}” is terminal repeat long; “ICP6” is a gene for a viral ribonucleotide reductase (vRR) for replication of wild-type HSV; “IR_{L}” is internal repeat long; “IRs” is internal repeat short; and “TR_{S}” is terminal repeat short.

FIG. 9C shows that, upon constructing the oHSV-IL2, vero cells were treated with the oHSV-IL2 for 24 hours. Supernatants were collected, and the IL-2 secretion levels measured are presented in FIG. 9C, and compared to IL-2 secretions measured in phosphate buffer solution (PBS), and IL-2 secretions measured from oHSV that is not constructed to express IL-2. FIG. 9D shows that the oncolytic virus does infect mouse glioblastoma cancer stem cells, showing images of mouse glioblastoma cancer stem cells treated with PBS (no cytopathic effect) for 24 hours and mouse glioblastoma stem cells treated with an oHSV-IL2 virus that express mCherry, also for 24 hours, with and without mCherry fluorescence. FIG. 9E shows the cytotoxicity of oHSV-IL2 in mouse glioblastoma stem cells by varying the multiplicity of infection (MOI) and performing an MTS assay of the glioblastoma stem cells after 4 days of infection with the virus. The dotted line shows viability of the mouse glioblastoma stem cells decreases.

In view of this data, one of skill can see that an oncolytic virus expressing IL-2 can be constructed, amplified in vero cells, and the IL-2 can be expressed from the virus in the vero cells. The oncolytic virus effectively infects glioblastoma stem cells, and the desired level of infection can be selected to kill the glioblastoma stem cells in a desired period of time after infection with the oncolytic virus. The dead stem cell will release stem cell antigens, release the oncolytic virus to kill tumor cells, and release the expressed IL-2 to kill additional stem cells. This method of treatment will create an immunological memory that will be sustained for a desired period of time, as well as package the virus and cytokine in a manner that reduces toxicity to the subject during administration of the live, cancer stem cells, and reduces the amount of virus and cytotoxin that needs to be administered to the subject, as the amplification of the virus and secretion of the cytokine occurs in the live, cancer stem cell.

### Example 10. Infection of 4T1 Cells with an Oncolytic Virus, Cytopathic Effects

This example shows that the oncolytic virus infects a variety of breast cancer cells, both mouse and human, and is cytotoxic to the cells, with or without expression of the cytokine IL12.

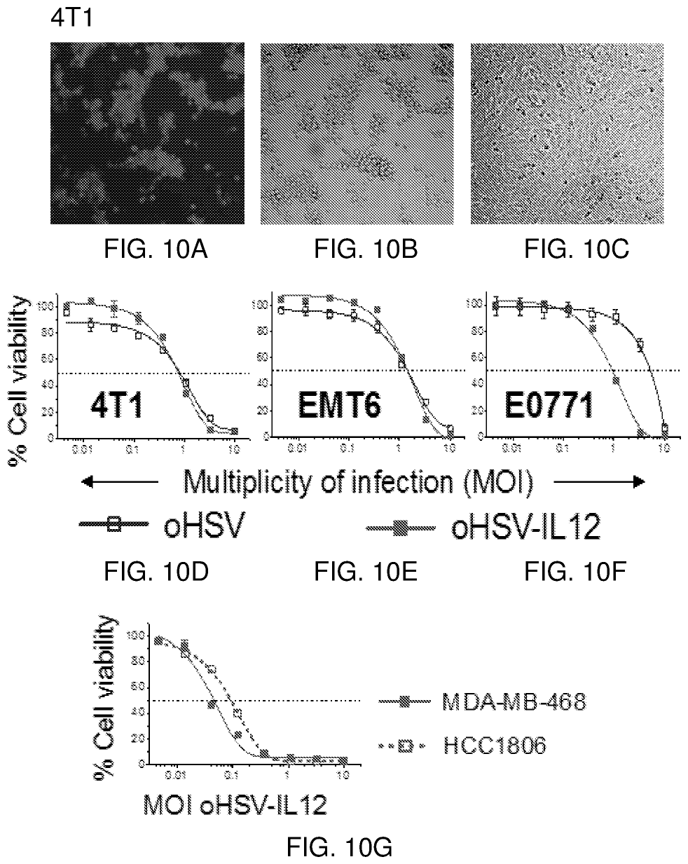

FIGS. 10A-10G show that the oncolytic virus does infect a variety of breast cancer cells, according to some embodiments. FIG. 10A shows the infection of the 4T1 cells for 36 hours through the release of mCherry in the cells through fluorescence imaging. FIG. 10B shows the same infection for 36 hours without the fluorescence imaging. FIG. 10C shows the 4T1 cells treated with PBS (no cytopathic effect) for 36 hours.

FIGS. 10D-10F show the cytotoxicity of oHSV-IL2 in 4T1, EMT6, and E0771 breast cancer cells, all of which are considered triple-negative breast cancer cells. Triple-negative breast cancer (TNBC) accounts for about 10-15% of all breast cancers. 4T1 is a breast cancer cell line derived from the mammary gland tissue of a mouse BALB/c strain. EMT6 (ATCC® CRL-2755Tm) is a murine mammary carcinoma cell line derived from a transplanted hyperplastic alveolar nodule of a BALB/c mouse. E0771 is a murine mammary cancer cell line originally isolated from a spontaneous tumour in C57BL/6 mouse. The figures show that both the oHSV-IL12 and the oHSV without expression of IL12 were both cytotoxic to the TNBC cells after 4 days of infection with the virus. FIG. 10G shows that the oHSV-IL12 was cytotoxic to human TNBC cells known as MDA-MB-468 and HCC1806. MDA-MB-468 is a cell line with epithelial morphology that was isolated from a pleural effusion of a 51-year-old Black female patient with metastatic adenocarcinoma of the breast. HCC1806 is an epithelial cell line isolated from the mammary gland of a 60-year-old, Black, female patient with acantholytic squamous cell carcinoma (ASCC), TNM Stage IIB, grade 2.

### Example 11. Dendritic Cells are Activated by Oncolytic Herpes Simplex Virus but More so with Expression of IL-12

The E0771 cell line is a spontaneously developing medullary breast adenocarcinoma from C57BL/6 mice, and the oncolytic virus expressing cytokines can increase the activation of dendritic cells. The activation is measured by an increase in cytokine production.

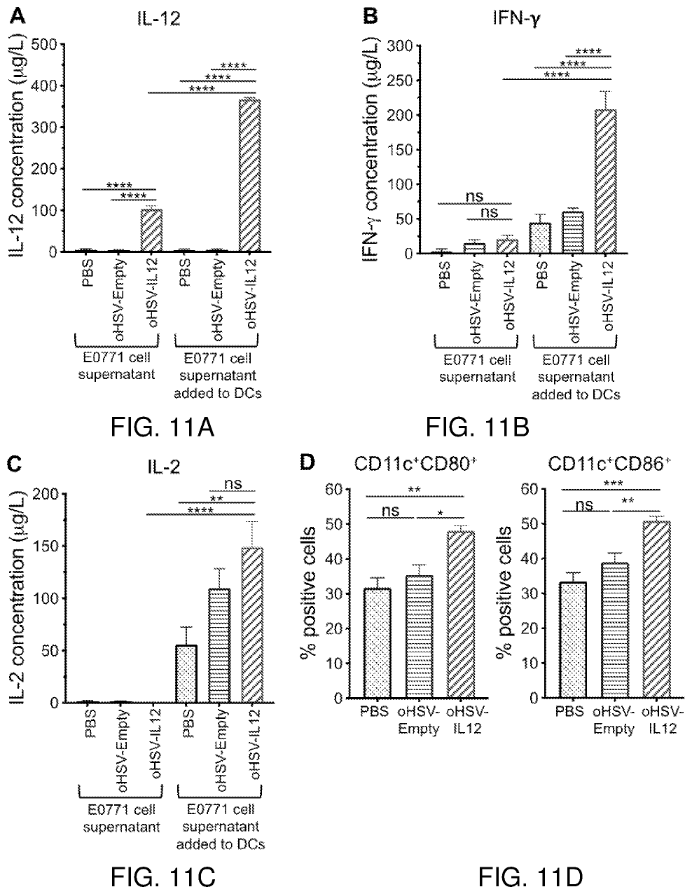

FIGS. 11A-11D illustrate that dendritic cells (DCs) produce cytokines and are susceptible to cytokine-mediated activation in response to treatment with a virus-infected cancer cell supernatant, according to some embodiments. In this example, we (i) treated E0771 cells with phosphate buffer solution (PBS), an oncolytic Herpes Simplex virus that does not express cytokine (oHSV-Empty), and an oncolytic Herpes Simplex virus that expresses IL-12 cytokine (oHSV-IL12); (ii) collected the cells with supernatant; (iii) freeze-thawed the cells; and (iv) measured the presence of cytokines IL-12, IFN-γ, and IL-2, as shown in FIGS. 11A-11C (bracketed as “E0771 cell supernatant”). We then isolated dendritic cells (DCs) from a naive mouse, UV irradiated the supernatants, and treated the dendritic cells (DCs) with the irradiated supernatants for 48 hours at 37° C. to activate the dendritic cells and measure cytokine production from the dendritic cells due to the treatment by the cytokines, as shown in FIGS. 11A-11C (bracketed as “E0771 cell supernatant added to DCs”). CD11c is a maker for dendritic cells. And, mature dendritic cells express CD80 and CD86 which is needed for T cell activation. FIG. 11D shows that the same DCs that were activated were stained with antibodies against CD11c, CD80, and CD86, and flow cytometry was used to analyze “% positive cells”. The data shows that the empty virus activated the dendritic cells, but the virus expressing the IL12 had the highest activation of the dendritic cells.

### Example 12. Activation of T Cells by an Oncolytic Heroes Simplex Virus Expressing IL-12 in an Orthotopic Model of Breast Cancer

EMT6 cells are epithelial cells isolated from the breast of a mouse with a mammary tumor. Orthotopic models involve implantation of tumor cell lines or patient-derived cell xenografts into animal tumor models into the organ or tissue which matches the tumor histotype. This creates a more disease-relevant environment for the assessment of tumor growth, which can be analyzed by optical imaging.

In this example, we implanted EMT6 cells in the right axillary mammary fat pad of BALB/c mice and treated the tumors on days 10, 14, and 17 with phosphate buffer solution (PBS) and oncolytic Herpes Simplex virus expressing IL-12 (oHSV-IL12), harvesting the cells from the mouse models on day 18 (PBS, n=5; oHSV-IL12, n=6).

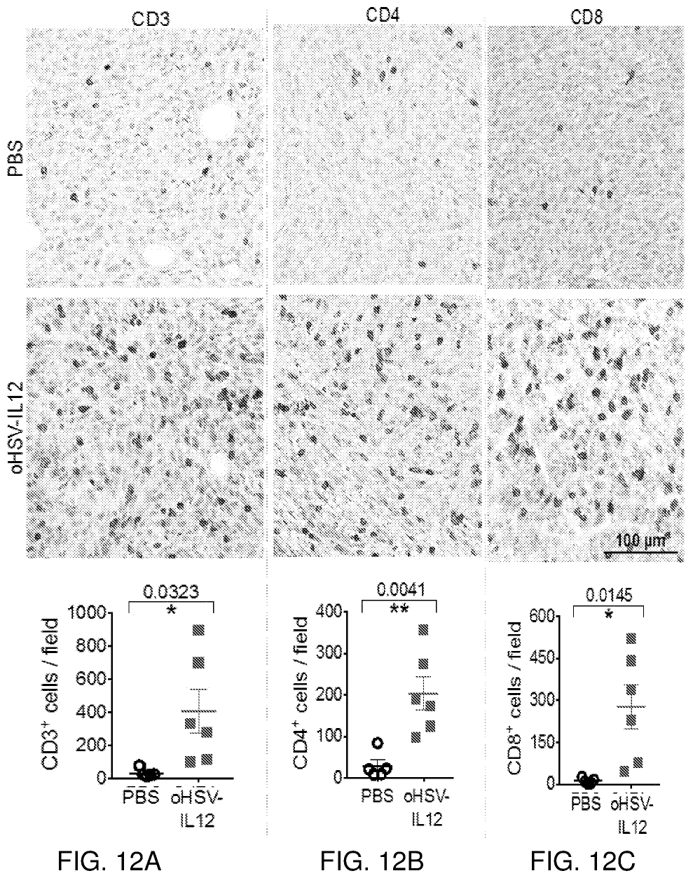

FIGS. 12A-12C show that the oncolytic virus expressing IL-12 increased the number of tumor-infiltrating T cells, namely CD3+, CD4+, and CD8+, according to some embodiments. FIG. 12A shows that the CD3+ T cells were increased. FIG. 12B shows that CD4+ T cells were increased. FIG. 12C shows that CD8+ T cells were increased.

On day 18, the harvested tumors were fixed in formalin, embedded in paraffin, sectioned (5 um in size), stained with primary antibodies against CD3, CD4, or CD8, and were then incubated with horseradish peroxidase-conjugated secondary antibodies, and immune cell antigens were visualized by applying DAB (3,3′-diaminobenzidine) substrate. Representative images of CD3, CD4, and CD8 positives, stained brown (20×). The bar is 100 um. The scatter plots shown the number of positive cells from each section counted from 8-9 random fields (one section from each mouse). The data are presented as Mean+/−SEM. Student's t test (2-tailed), *p<0.05; **p<0.01.

### Example 13. T-Cell Memory is a Critical Component of Immune Response: Central Memory T Cells Increase with Administration of an Oncolytic Virus Expressing IL-2

This example shows that central memory T cells increase in an orthotopic breast cancer mouse model when administering an oncolytic virus expressing IL-2 to an EMT6 orthotopic mouse model of breast cancer. EMT6 (ATCC® CRL-2755Tm) is a murine mammary carcinoma cell line derived from a transplanted hyperplastic alveolar nodule of a BALB/c mouse. The mice used in this experiment were BALB/c mice.

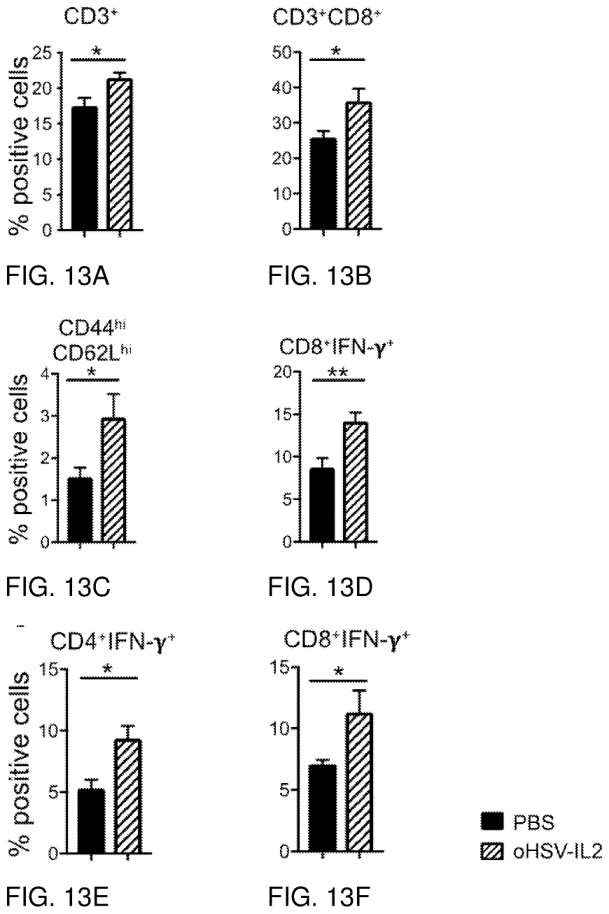

Established orthotopic mouse mammary tumors in the BALB/c mice were given intratumoral phosphate-buffered solution (PBS) or oHSV-IL2 (n=4) at 2-3 day intervals starting from day 5 to day 20 after tumor implantation. On day 21, tumors and spleens were harvested, stained with anti-mouse antibodies against CD45, CD3, CD4, CD8, CD44, CD62L, and IFN-γ, and Amcyan (live/dead). Flow cytometry was used to detect the T cells in FIGS. 13A-13F.

FIGS. 13A-13F illustrate how the administration of an oncolytic virus that expresses IL-2 increased T cell activity in tumors and spleens harvested from BALB/c mice having orthotopic mouse mammary tumors, including an increase in tumor infiltrating T cells, central memory T cells, and cytotoxic T cells from administration of the IL-2 expressing oncolytic virus to the mice, according to some embodiments. FIG. 13A shows an increase in tumor-infiltrating CD3⁺ T cells in tumors, and FIG. 13B shows an increase in tumor-infiltrating CD3⁺CD8⁺ T cells in tumors. FIG. 13C shows an increase in central memory CD44^{hi}CD62L^{hi} T cells in tumors, and FIG. 13D shows an increase in cytotoxic CD8⁺IFN-γ T cells in tumors. FIG. 13 E shows an increase in cytotoxic CD4⁼IFN-γ⁺ T cells in spleens, and FIG. 13F shows an increase in cytotoxic CD8⁺IFN-γ⁺ T cells in spleens.

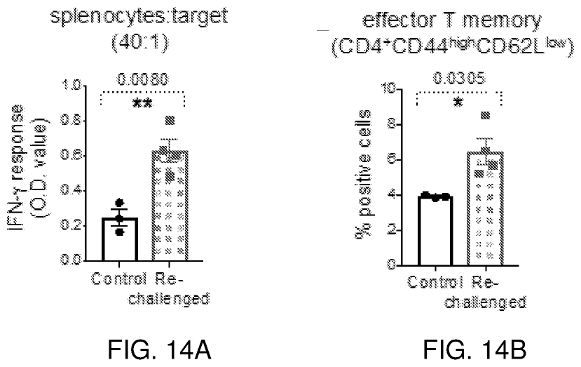

FIGS. 14A and 14B show an increase in IFN-γ and number of effector memory T cells when splenocytes taken from protected mice were re-challenged by being co-cultured with EMT6 cells, according to some embodiments. Here, n=4; the mice receiving the oHSV-IL2 oncolytic virus. The ratio of the splenocytes to target was 40:1, and the effector memory T cells were CD4⁺CD449^{high}CD62L^{low}. The control mice were age-matched tumor-bearing BALB/c mis (n=3).

The splenocytes were co-cultured with EMT6 cells for 48 hours and IFN-γ levels were measured in the supernatant. The splenocytes of protected and control mice were stained with antibodies against CE45, CD3, CD4, CD44, and CD62L, and Amcyan (live/dead), and analyzed by flow cytometry. The data are presented as mean+/−SEM. An unpaired Student's t test was used (2-tailed) in the analysis of the significance of the data.

One of skill will appreciate that this mouse model data shows (i) how to make an oncolytic virus, and that is reasonable that the systems and methods of treatment herein will provide (ii) an expression of the virus from the cancer stem cell that is cytotoxic to tumor cells with or without the additional expression of a desired protein, such as the IL2, (iii) an immune response will be induced, and (iv) an immune response memory will be induced.

### Example 14. Constructing a System of a Cancer Stem Cells Carrying an Oncolytic Virus

In view of the teachings herein, the skilled artisan will appreciate that any cancer stem cell can be used to carry the oncolytic viruses that express a desired protein for the tiple-killer effect. Any method of loading a cell with a virus can be used to load cancer stem cells. Moreover, there are a wide variety of oncolytic viruses that can be constructed and used, and the oHSV viruses used herein are merely examples of the several types of oncolytic viruses that can be used to transfect a cancer stem cell. Oncolytic viruses can also include, for example, but are not limited to, Herpes Simplex Virus (HSV), Newcastle Disease Virus (NDV), Vesicular Stomatitis Virus (VSV), Reovirus, Measles Virus, Retrovirus, Influenza Virus, Sindbis Virus, Vaccinia Virus, and Adenovirus.

Each of the viruses involve a cell death mechanism selected from the group consisting of apoptosis, angiogenesis, necrosis, and the like. Upon entering the tumor cell, the virus starts replicating and proliferates until the cell is annihilated by the bursting of the host cell membrane and release of the virus particles, which spread to adjacent tumor cells. When all of the tumor cells are destroyed, the virus can no longer replicate (as it cannot propagate in normal cells), whereupon it is cleared from the body by the immune system. Importantly, In addition to exerting a cytotoxic effect on the cells that they invade, oncolytic viruses can also stimulate immune responses. These responses can be driven by cytokine release or through an adaptive response to tumor-associated antigens, for example. In some cases, the oncolytic virus can stimulate secretion of IFN-α, interleukins and tumor necrosis factor (TNF)-α, which then can activate dendritic cells, NK cells and T cells. These in turn enhance a tumor-specific immune response toward tumor cell targets that is distinct from cell death imposed by the virus itself.

### Example 15. Designing Viruses with Immune Stimulatory Molecules for Loading Cancer Stem Cells

As can be seen from the teachings set-forth above, any of the viruses taught herein can be designed to comprise one or more genes encoding an immune stimulatory molecule. As such the cancer stem cells loaded with a virus expressing the immune stimulatory molecule express proteins that help facilitate an immune response by acting as either an agonist of the immune response or an antagonist of the activity of an immune response inhibitor. In some embodiments, the expressed protein can inhibit the activity of RNA molecules (e.g. shRNA, antisense RNA, RNAi or micro RNA) that inhibit the expression of immune inhibitory molecules.

In some embodiments, the immune stimulatory molecules can include the IL-2, IL12, IL-15, IL-18, and IL-21 proteins discussed above. However, they can also include human and mouse CD40 ligands and encoding nucleotides (SEQ ID NO: 66 to SEQ ID NO: 75), human and mouse 4-1-BB ligands and encoding nucleotides (SEQ ID NO: 76 to SEQ ID NO: 83), human and mouse GITR ligands and encoding nucleotides (SEQ ID NO: 84 to SEQ ID NO: 91), human and mouse OX40 ligands and encoding nucleotides (SEQ ID NO: 92 to SEQ ID NO: 99), and human and mouse ICOS ligands and encoding nucleotides (SEQ ID NO: 100 to SEQ ID NO: 112).

In some embodiments, the immune stimulatory molecules can include the fit3 ligand, type I interferons, including the interferon alpha and interferon beta, and interferon gamma discussed above. In some embodiments, the immune stimulatory molecules can include IL-24, type III interferon (IL-28, IL-29), as well as other cytokines discussed above such as TNF alpha or GM-CSF. In some embodiments, the immune stimulatory molecules can include TGF beta, or immune checkpoint antagonists Immune checkpoint antagonists that include antibodies. In some embodiments, the immune stimulatory approaches can include the use of single chain antibodies and RNA1/siRNA/microRNA/antisense RNA knockdown approaches.

In some embodiments, the agonists of immune potentiating/co-stimulatory pathways include mutant or wild type, soluble, secreted and/or membrane bound ligands, and agonistic antibodies. In some embodiments, the expressed protein targets immune co-inhibitory or immune co-stimulatory pathways. Such proteins can include, for example, CTLA-4 (antagonist), PD-1 (antagonist), PD-L1 (antagonist), LAG-3 (antagonist), TIM-3 (antagonist), VISTA (antagonist), CSF1R (antagonist), IDO (antagonist), CEACAM1 (antagonist), GITR (agonist), 4-1-BB (agonist), KIR (antagonist), SLAMF7 (antagonist), OX40 (agonist), CD40 (agonist), ICOS (agonist) or CD47 (antagonist)

In some embodiments, a CTLA-4 inhibitor can be used. The CTLA-4 inhibitor can be an antibody or an antigen binding fragment thereof. The term “antibody” can refer to whole antibodies, an antigen binding fragment (i.e., “antigen-binding portion”), or a single chain thereof. In some embodiments, the antibody is a glycoprotein having at least two heavy (H) chains and two light (kappa)(L) chains inter-connected by disulfide bonds, or an antigen binding portion thereof. While not intending to be bound by any theory or mechanism of action, each heavy chain can have a heavy chain variable region (abbreviated herein as VH) and a heavy chain constant region. Likewise, each light chain can have a light chain variable region (abbreviated herein as VL) and a light chain constant region. The variable regions of the heavy and light chains contain a binding domain that interacts with an antigen. The constant regions of the heavy and light chains can mediate the binding of the immunoglobulin to host tissues or factors, including various cells of the immune system (e.g., effector cells) and the first component (Clq) of the classical complement system.

In some embodiments, the antibody is a monoclonal antibody. In some embodiments, the antibody is a chimeric antibody. In some embodiments, the antibody is a humanised antibody or a human antibody.

The term “antigen-binding fragment” refers to one or more fragments of an antibody that retain the ability to specifically bind to CTLA-4. The antigen-binding fragment can have the ability to inhibit CTLA-4 to at least inhibit the CTLA-4 blockade of a stimulatory immune response. Examples of suitable fragments that can be used include a Fab fragment, a F(ab′)2 fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are included within the term “antigen-binding portion” of an antibody. In some embodiments, the scFv molecule can be selected from the teachings of, for example, WO2007/123737 and WO2014/066532, each teachings of which are incorporated herein in their entirety by reference. The scFv may be encoded, for example, by the nucleotide sequence shown in SEQ ID NO: 114 in some embodiments, or the nucleotide sequence shown in SEQ ID NO:116 in some embodiments.

One of skill will appreciate that a virus loaded into the cancer stem cells taught herein can be designed to express one or more desired proteins. In some embodiments, the virus can be designed to express 1, 2, 3 or 4 desired proteins. Moreover, it should also be appreciated that any of the nucleotide sequences used to express a desired protein can be codon optimized so as to increase expression levels over that of the unoptimized sequence.

### Example 16. Determining the Administration Amount of Cancer Stem Cells, Cancer Stem Cells Loaded with Oncolytic Virus, or Cancer Stem Cells Loaded with Oncolytic Virus Expressing a Desired Protein

One of skill will appreciate that the effectiveness of a treatment will depend, at least in part, on the amount of reprogrammed cancer stem cells that are delivered, as the cancer stem cell count administered provides a measure of antigen released, oncolytic virus released, and/or desired protein expressed in the subject. The amounts administered can be expressed “cells per dose” (based on an average 75 kg body weight herein) or “cells/kg of body weight per dose” Amount of cancer stem cells administered is a measure of the amount of antigen, oncolytic virus, and/or desired amount of protein administered

This example further elaborate on the amount of live, reprogrammed cancer stem cells that can be administered, in some embodiments. In some embodiments, the amount of live reprogrammed cancer stem cells that can be administered in a single dose can range from about 0.1×10⁶ to about 500×10⁶ cells per dose, or any range or amount therein in increments of 0.01×10⁶ cells per dose, to obtain the desired antigen immune response, the desired prophylactic response, and/or the desired treatment response following the death of the reprogrammed cancer stem cells in the subject.

In some embodiments, the amount of live reprogrammed cancer stem cells that can be administered in a single dose can range from 1×10⁶ to 10×10⁶ cells per dose, from 1×10⁶ to 15×10⁶ cells per dose, from 1×10⁶ to 20×10⁶ cells per dose, from 1×10⁶ to 25×10⁶ cells per dose, from 1×10⁶ to 30×10⁶ cells per dose, from 1×10⁶ to 35×10⁶ cells per dose, from 1×10⁶ to 40×10⁶ cells per dose, from 1×10⁶ to 45×10⁶ cells per dose, from 1×10⁶ to 50×10⁶ cells per dose, from 1×10⁶ to 55×10⁶ cells per dose, from 1×10⁶ to 60×10⁶ cells per dose, from 1×10⁶ to 65×10⁶ cells per dose, from 1×10⁶ to 70×10⁶ cells per dose, from 1×10⁶ to 75×10⁶ cells per dose, from 1×10⁶ to 80×10⁶ cells per dose, from 1×10⁶ to 85×10⁶ cells per dose, from 1×10⁶ to 90×10⁶ cells per dose, from 1×10⁶ to 95×10⁶ cells per dose, from 1×10⁶ to 100×10⁶ cells per dose, or any range or amount therein in increments of 1×10⁶ cells per dose.

In some embodiments, the amount of live reprogrammed cancer stem cells that can be administered in a single dose can range from 5×10⁶ to 10×10⁶ cells per dose, from 5×10⁶ to 15×10⁶ cells per dose, from 5×10⁶ to 20×10⁶ cells per dose, from 5×10⁶ to 25×10⁶ cells per dose, from 5×10⁶ to 30×10⁶ cells per dose, from 5×10⁶ to 35×10⁶ cells per dose, from 5×10⁶ to 40×10⁶ cells per dose, from 5×10⁶ to 45×10⁶ cells per dose, from 5×10⁶ to 50×10⁶ cells per dose, from 5×10⁶ to 55×10⁶ cells per dose, from 5×10⁶ to 60×10⁶ cells per dose, from 5×10⁶ to 65×10⁶ cells per dose, from 5×10⁶ to 70×10⁶ cells per dose, from 5×10⁶ to 75×10⁶ cells per dose, from 5×10⁶ to 80×10⁶ cells per dose, from 5×10⁶ to 85×10⁶ cells per dose, from 5×10⁶ to 90×10⁶ cells per dose, from 5×10⁶ to 95×10⁶ cells per dose, from 5×10⁶ to 100×10⁶ cells per dose, or any range or amount therein in increments of 1×10⁶ cells per dose.

In some embodiments, the amount of live reprogrammed cancer stem cells that can be administered in a single dose can range from 10×10⁶ to 15×10⁶ cells per dose, from 10×10⁶ to 20×10⁶ cells per dose, from 10×10⁶ to 25×10⁶ cells per dose, from 10×10⁶ to 30×10⁶ cells per dose, from 10×10⁶ to 35×10⁶ cells per dose, from 10×10⁶ to 40×10⁶ cells per dose, from 10×10⁶ to 45×10⁶ cells per dose, from 10×10⁶ to 50×10⁶ cells per dose, from 10×10⁶ to 55×10⁶ cells per dose, from 10×10⁶ to 60×10⁶ cells per dose, from 10×10⁶ to 65×10⁶ cells per dose, from 10×10⁶ to 70×10⁶ cells per dose, from 10×10⁶ to 75×10⁶ cells per dose, from 10×10⁶ to 80×10⁶ cells per dose, from 10×10⁶ to 85×10⁶ cells per dose, from 10×10⁶ to 90×10⁶ cells per dose, from 10×10⁶ to 95×10⁶ cells per dose, from 10×10⁶ to 100×10⁶ cells per dose, or any range or amount therein in increments of 1×10⁶ cells per dose.

In some embodiments, the amount of live reprogrammed cancer stem cells that can be administered in a single dose can range from 20×10⁶ to 25×10⁶ cells per dose, from 20×10⁶ to 30×10⁶ cells per dose, from 20×10⁶ to 35×10⁶ cells per dose, from 20×10⁶ to 40×10⁶ cells per dose, from 20×10⁶ to 45×10⁶ cells per dose, from 20×10⁶ to 50×10⁶ cells per dose, from 20×10⁶ to 55×10⁶ cells per dose, from 20×10⁶ to 60×10⁶ cells per dose, from 20×10⁶ to 65×10⁶ cells per dose, from 20×10⁶ to 70×10⁶ cells per dose, from 20×10⁶ to 75×10⁶ cells per dose, from 20×10⁶ to 80×10⁶ cells per dose, from 20×10⁶ to 85×10⁶ cells per dose, from 20×10⁶ to 90×10⁶ cells per dose, from 20×10⁶ to 95×10⁶ cells per dose, from 20×10⁶ to 100×10⁶ cells per dose, or any range or amount therein in increments of 1×10⁶ cells per dose.

In some embodiments, the amount of live reprogrammed cancer stem cells that can be administered in a single dose can range from 30×10⁶ to 35×10⁶ cells per dose, from 30×10⁶ to 40×10⁶ cells per dose, from 30×10⁶ to 45×10⁶ cells per dose, from 30×10⁶ to 50×10⁶ cells per dose, from 30×10⁶ to 55×10⁶ cells per dose, from 30×10⁶ to 60×10⁶ cells per dose, from 30×10⁶ to 65×10⁶ cells per dose, from 30×10⁶ to 70×10⁶ cells per dose, from 30×10⁶ to 75×10⁶ cells per dose, from 30×10⁶ to 80×10⁶ cells per dose, from 30×10⁶ to 85×10⁶ cells per dose, from 30×10⁶ to 90×10⁶ cells per dose, from 30×10⁶ to 95×10⁶ cells per dose, from 30×10⁶ to 100×10⁶ cells per dose, or any range or amount therein in increments of 1×10⁶ cells per dose.

In some embodiments, the amount of live reprogrammed cancer stem cells that can be administered in a single dose can range from 40×10⁶ to 45×10⁶ cells per dose, from 40×10⁶ to 50×10⁶ cells per dose, from 40×10⁶ to 55×10⁶ cells per dose, from 40×10⁶ to 60×10⁶ cells per dose, from 40×10⁶ to 65×10⁶ cells per dose, from 40×10⁶ to 70×10⁶ cells per dose, from 40×10⁶ to 75×10⁶ cells per dose, from 40×10⁶ to 80×10⁶ cells per dose, from 40×10⁶ to 85×10⁶ cells per dose, from 40×10⁶ to 90×10⁶ cells per dose, from 40×10⁶ to 95×10⁶ cells per dose, from 40×10⁶ to 100×10⁶ cells per dose, or any range or amount therein in increments of 1×10⁶ cells per dose.

In some embodiments, the amount of live reprogrammed cancer stem cells that can be administered in a single dose can range from 50×10⁶ to 55×10⁶ cells per dose, from 50×10⁶ to 60×10⁶ cells per dose, from 50×10⁶ to 65×10⁶ cells per dose, from 50×10⁶ to 70×10⁶ cells per dose, from 50×10⁶ to 75×10⁶ cells per dose, from 50×10⁶ to 80×10⁶ cells per dose, from 50×10⁶ to 85×10⁶ cells per dose, from 50×10⁶ to 90×10⁶ cells per dose, from 50×10⁶ to 95×10⁶ cells per dose, from 50×10⁶ to 100×10⁶ cells per dose, or any range or amount therein in increments of 1×10⁶ cells per dose.

In some embodiments, the amount of live reprogrammed cancer stem cells that can be administered in a single dose can range from 60×10⁶ to 65×10⁶ cells per dose, from 60×10⁶ to 70×10⁶ cells per dose, from 60×10⁶ to 75×10⁶ cells per dose, from 60×10⁶ to 80×10⁶ cells per dose, from 60×10⁶ to 85×10⁶ cells per dose, from 60×10⁶ to 90×10⁶ cells per dose, from 60×10⁶ to 95×10⁶ cells per dose, from 60×10⁶ to 100×10⁶ cells per dose, or any range or amount therein in increments of 1×10⁶ cells per dose.

In some embodiments, the amount of live reprogrammed cancer stem cells that can be administered in a single dose can range from 70×10⁶ to 75×10⁶ cells per dose, from 70×10⁶ to 80×10⁶ cells per dose, from 70×10⁶ to 85×10⁶ cells per dose, from 70×10⁶ to 90×10⁶ cells per dose, from 70×10⁶ to 95×10⁶ cells per dose, from 70×10⁶ to 100×10⁶ cells per dose, or any range or amount therein in increments of 1×10⁶ cells per dose.

In some embodiments, the amount of live reprogrammed cancer stem cells that can be administered in a single dose can range from 80×10⁶ to 85×10⁶ cells per dose, from 80×10⁶ to 90×10⁶ cells per dose, from 80×10⁶ to 95×10⁶ cells per dose, from 80×10⁶ to 100×10⁶ cells per dose, or any range or amount therein in increments of 1×10⁶ cells per dose.

In some embodiments, the amount of live reprogrammed cancer stem cells that can be administered in a single dose can range from 90×10⁶ to 95×10⁶ cells per dose, from 90×10⁶ to 100×10⁶ cells per dose, or any range or amount therein in increments of 1×10⁶ cells per dose.

In some embodiments, the amount of live reprogrammed cancer stem cells that can be administered in a single dose can range from 100×10⁶ to 300×10⁶ cells per dose, from 110×10⁶ to 300×10⁶ cells per dose, from 120×10⁶ to 300×10⁶ cells per dose, from 130×10⁶ to 300×10⁶ cells per dose, from 140×10⁶ to 300×10⁶ cells per dose, from 150×10⁶ to 300×10⁶ cells per dose, from 160×10⁶ to 300×10⁶ cells per dose, from 170×10⁶ to 300×10⁶ cells per dose, from 180×10⁶ to 300×10⁶ cells per dose, from 190×10⁶ to 300×10⁶ cells per dose, from 200×10⁶ to 300×10⁶ cells per dose, from 210×10⁶ to 300×10⁶ cells per dose, from 220×10⁶ to 300×10⁶ cells per dose, from 230×10⁶ to 300×10⁶ cells per dose, from 240×10⁶ to 300×10⁶ cells per dose, from 250×10⁶ to 300×10⁶ cells per dose, from 260×10⁶ to 300×10⁶ cells per dose, from 270×10⁶ to 300×10⁶ cells per dose, from 280×10⁶ to 300×10⁶ cells per dose, from 290×10⁶ to 300×10⁶ cells per dose, or any range or amount therein in increments of 1×10⁶ cells per dose.

In some embodiments, the amount of live reprogrammed cancer stem cells that can be administered in a single dose can range from 200×10⁶ to 400×10⁶ cells per dose, from 210×10⁶ to 400×10⁶ cells per dose, from 220×10⁶ to 400×10⁶ cells per dose, from 230×10⁶ to 400×10⁶ cells per dose, from 240×10⁶ to 400×10⁶ cells per dose, from 250×10⁶ to 400×10⁶ cells per dose, from 260×10⁶ to 400×10⁶ cells per dose, from 270×10⁶ to 400×10⁶ cells per dose, from 280×10⁶ to 400×10⁶ cells per dose, from 290×10⁶ to 400×10⁶ cells per dose, 300×10⁶ to 400×10⁶ cells per dose, from 310×10⁶ to 400×10⁶ cells per dose, from 320×10⁶ to 400×10⁶ cells per dose, from 330×10⁶ to 400×10⁶ cells per dose, from 340×10⁶ to 400×10⁶ cells per dose, from 350×10⁶ to 400×10⁶ cells per dose, from 360×10⁶ to 400×10⁶ cells per dose, from 370×10⁶ to 400×10⁶ cells per dose, from 380×10⁶ to 400×10⁶ cells per dose, from 390×10⁶ to 400×10⁶ cells per dose, or any range or amount therein in increments of 1×10⁶ cells per dose.

In some embodiments, the amount of live reprogrammed cancer stem cells that can be administered in a single dose can range from 300×10⁶ to 500×10⁶ cells per dose, from 310×10⁶ to 500×10⁶ cells per dose, from 320×10⁶ to 500×10⁶ cells per dose, from 330×10⁶ to 500×10⁶ cells per dose, from 340×10⁶ to 500×10⁶ cells per dose, from 350×10⁶ to 500×10⁶ cells per dose, from 360×10⁶ to 500×10⁶ cells per dose, from 370×10⁶ to 500×10⁶ cells per dose, from 380×10⁶ to 500×10⁶ cells per dose, from 390×10⁶ to 500×10⁶ cells per dose, from 400×10⁶ to 500×10⁶ cells per dose, from 410×10⁶ to 500×10⁶ cells per dose, from 420×10⁶ to 500×10⁶ cells per dose, from 430×10⁶ to 500×10⁶ cells per dose, from 440×10⁶ to 500×10⁶ cells per dose, from 450×10⁶ to 500×10⁶ cells per dose, from 460×10⁶ to 500×10⁶ cells per dose, from 470×10⁶ to 500×10⁶ cells per dose, from 480×10⁶ to 500×10⁶ cells per dose, from 490×10⁶ to 500×10⁶ cells per dose, or any range or amount therein in increments of 1×10⁶ cells per dose.

Likewise, the amount of live, reprogrammed cancer stem cells administered can be selected in terms of the body weight of the subject. In some embodiments, the amount of live reprogrammed cancer stem cells that can be administered in a single dose can range from 0.01×10⁶ to 10.0×10⁶ cells/kg of body weight per dose, from 0.1×10⁶ to 10.0×10⁶ cells/kg of body weight per dose, from 1.0×10⁶ to 10.0×10⁶ cells/kg of body weight per dose, from 0.01×10⁶ to 5.0×10⁶ cells/kg of body weight per dose, from 0.1×10⁶ to 5.0×10⁶ cells/kg of body weight per dose, from 1.0×10⁶ to 5.0×10⁶ cells/kg of body weight per dose, or any amount or range therein in increments of 0.01×10⁶ cells/kg of body weight per dose, to obtain the desired antigen immune response, the desired prophylactic response, and/or the desired treatment response following the death of the reprogrammed cancer stem cells in the subject.

In some embodiments, the amount of live reprogrammed cancer stem cell are administered in an amount of 0.1×10⁶ cells/kg body weight per dose, 0.2×10⁶ cells/kg body weight per dose, 0.3×10⁶ cells/kg body weight per dose, 0.4×10⁶ cells/kg body weight per dose, 0.5×10⁶ cells/kg body weight per dose, 0.6×10⁶ cells/kg body weight per dose, 0.7×10⁶ cells/kg body weight per dose, 0.8×10⁶ cells/kg body weight per dose, 0.9×10⁶ cells/kg body weight per dose, 1.0×10⁶ cells/kg body weight per dose, 1.1×10⁶ cells/kg body weight per dose, 1.2×10⁶ cells/kg body weight per dose, 1.3×10⁶ cells/kg body weight per dose, 1.4×10⁶ cells/kg body weight per dose, 1.5×10⁶ cells/kg body weight per dose, 1.6×10⁶ cells/kg body weight per dose, 1.7×10⁶ cells/kg body weight per dose, 1.8×10⁶ cells/kg body weight per dose, 1.9×10⁶ cells/kg body weight per dose, 2.0×10⁶ cells/kg body weight per dose, 2.1×10⁶ cells/kg body weight per dose, 2.2×10⁶ cells/kg body weight per dose, 2.3×10⁶ cells/kg body weight per dose, 2.4×10⁶ cells/kg body weight per dose, 2.5×10⁶ cells/kg body weight per dose, 2.6×10⁶ cells/kg body weight per dose, 2.7×10⁶ cells/kg body weight per dose, 2.8×10⁶ cells/kg body weight per dose, 2.9×10⁶ cells/kg body weight per dose, 3.0×10⁶ cells/kg body weight per dose, 3.1×10⁶ cells/kg body weight per dose, 3.2×10⁶ cells/kg body weight per dose, 3.3×10⁶ cells/kg body weight per dose, 3.4×10⁶ cells/kg body weight per dose, 3.5×10⁶ cells/kg body weight per dose, 3.6×10⁶ cells/kg body weight per dose, 3.7×10⁶ cells/kg body weight per dose, 3.8×10⁶ cells/kg body weight per dose, 3.9×10⁶ cells/kg body weight per dose, 4.0×10⁶ cells/kg body weight per dose, 4.1×10⁶ cells/kg body weight per dose, 4.2×10⁶ cells/kg body weight per dose, 4.3×10⁶ cells/kg body weight per dose, 4.4×10⁶ cells/kg body weight per dose, 4.5×10⁶ cells/kg body weight per dose, 4.6×10⁶ cells/kg body weight per dose, 4.7×10⁶ cells/kg body weight per dose, 4.8×10⁶ cells/kg body weight per dose, 4.9×10⁶ cells/kg body weight per dose, 5.0×10⁶ cells/kg body weight per dose, 5.1×10⁶ cells/kg body weight per dose, 5.2×10⁶ cells/kg body weight per dose, 5.3×10⁶ cells/kg body weight per dose, 5.4×10⁶ cells/kg body weight per dose, 5.5×10⁶ cells/kg body weight per dose, 5.6×10⁶ cells/kg body weight per dose, 5.7×10⁶ cells/kg body weight per dose, 5.8×10⁶ cells/kg body weight per dose, 5.9×10⁶ cells/kg body weight per dose, 6.0×10⁶ cells/kg body weight per dose, 6.1×10⁶ cells/kg body weight per dose, 6.2×10⁶ cells/kg body weight per dose, 6.3×10⁶ cells/kg body weight per dose, 6.4×10⁶ cells/kg body weight per dose, 6.5×10⁶ cells/kg body weight per dose, 6.6×10⁶ cells/kg body weight per dose, 6.7×10⁶ cells/kg body weight per dose, 6.8×10⁶ cells/kg body weight per dose, 6.9×10⁶ cells/kg body weight per dose, 7.0×10⁶ cells/kg body weight per dose, 7.1×10⁶ cells/kg body weight per dose, 7.2×10⁶ cells/kg body weight per dose, 7.3×10⁶ cells/kg body weight per dose, 7.4×10⁶ cells/kg body weight per dose, 7.5×10⁶ cells/kg body weight per dose, 7.6×10⁶ cells/kg body weight per dose, 7.7×10⁶ cells/kg body weight per dose, 7.8×10⁶ cells/kg body weight per dose, 7.9×10⁶ cells/kg body weight per dose, 8.0×10⁶ cells/kg body weight per dose, 8.1×10⁶ cells/kg body weight per dose, 8.2×10⁶ cells/kg body weight per dose, 8.3×10⁶ cells/kg body weight per dose, 8.4×10⁶ cells/kg body weight per dose, 8.5×10⁶ cells/kg body weight per dose, 8.6×10⁶ cells/kg body weight per dose, 8.7×10⁶ cells/kg body weight per dose, 8.8×10⁶ cells/kg body weight per dose, 8.9×10⁶ cells/kg body weight per dose, 9.0×10⁶ cells/kg body weight per dose, 9.1×10⁶ cells/kg body weight per dose, 9.2×10⁶ cells/kg body weight per dose, 9.3×10⁶ cells/kg body weight per dose, 9.4×10⁶ cells/kg body weight per dose, 9.5×10⁶ cells/kg body weight per dose, 9.6×10⁶ cells/kg body weight per dose, 9.7×10⁶ cells/kg body weight per dose, 9.8×10⁶ cells/kg body weight per dose, 9.9×10⁶ cells/kg body weight per dose, 10.0×10⁶ cells/kg body weight per dose, or any range or amount herein in increments 0.05 cells per dose.

In some embodiments, the amount of live reprogrammed cancer stem cells that can be administered in a single dose can range from 0.01×10⁶ to 5.0×10⁶ cells/kg of body weight per dose, from 0.02×10⁶ to 4.0×10⁶ cells/kg of body weight per dose, from 0.03×10⁶ to 3.0×10⁶ cells/kg of body weight per dose, from 0.04×10⁶ to 2.0×10⁶ cells/kg of body weight per dose, from 0.05×10⁶ to 1.0×10⁶ cells/kg of body weight per dose.

In some embodiments, the amount of live reprogrammed cancer stem cells that can be administered in a single dose can range from 0.10×10⁶ to 5.0×10⁶ cells/kg of body weight per dose, from 0.20×10⁶ to 4.0×10⁶ cells/kg of body weight per dose, from 0.30×10⁶ to 3.0×10⁶ cells/kg of body weight per dose, from 0.40×10⁶ to 2.0×10⁶ cells/kg of body weight per dose, from 0.50×10⁶ to 1.0×10⁶ cells/kg of body weight per dose.

In some embodiments, the amount of live reprogrammed cancer stem cells that can be administered in a single dose can range from 0.01×10⁶ to 0.5×10⁶ cells/kg of body weight per dose, 0.5×10⁶ to 1.0×10⁶ cells/kg of body weight per dose, 1.0×10⁶ to 1.5×10⁶ cells/kg of body weight per dose, from 1.5×10⁶ to 2.0×10⁶ cells/kg of body weight per dose, from 2.0×10⁶ to 2.5×10⁶ cells/kg of body weight per dose, from 2.5×10⁶ to 3.0×10⁶ cells/kg of body weight per dose, from 3.0×10⁶ to 3.5×10⁶ cells/kg of body weight per dose, from 3.5×10⁶ to 4.0×10⁶ cells/kg of body weight per dose, from 4.0×10⁶ to 4.5×10⁶ cells/kg of body weight per dose, from 4.5×10⁶ to 5.0×10⁶ cells/kg of body weight per dose, or any range or amount herein in increments 0.05/kg cells per dose.

Loading Cancer Stem Cells with Virus

Any known method of loading a cancer stem cell can be used. In some embodiments, the loading of the cancer stem cell can be a transfection which is stable or transient. In some embodiments, the transfection is transient and produces a desired titer within 12 hrs, 24 hrs, 36 hrs, 48 hrs, 60 hrs, 72 hrs, 84 hrs, 96 hrs, 5 days, 6 days, 7 days, 8 days, 9 days, 10 days, 11 days, 12 days, or any range or amount therein in increments of 1 hr.

One of skill will appreciate that several loading techniques can be used to load the cancer stem cells with an oncolytic virus, including chemical or mechanical loading methods. The method is used for a successful loading of at least 1 oncolytic virus taught herein into a cancer stem cell. The goal is to load successfully lead each cancer stem cell that will be administered to a subject with at least 1 oncolytic virus taught herein. For example, if 20×10⁶ cancer stem cells are administered as a dose to a subject, then 20×10⁶ viruses are loaded into that dose, assuming the loading of 1 virus per cancer stem cell.

Any known method can be used to load a cancer stem cell. In some embodiments, a cationic polymer transfection can be used. In some embodiments, a high intensity near-IR laser pulse can be used to perforate the cell membrane. In some embodiments, electroporation can be used to transfect the cancer stem cells. In some embodiments, liposomes can be used to transfect the cancer stem cells, transporting the oncolytic virus into the cell by endocytosis, releasing the virus into cytoplasm. In some embodiments, chemical transfection can be used to transfect the cancer stem cells, including calcium phosphate precipitation that carries the oncolytic virus in a calcium chloride solution that forms an insoluble calcium phosphate precipitate with virus on it's surface that is mixed with the cancer stem cells for the transfection. Other transfection methods include sonoporation to induce permeation and transfection, cell squeezing to induce transfection, impalefection to insert nanofibers carrying the virus, optical transfection with a laser, protoplast fusion with a lysozyme, magnetofaction with nanoparticles combined with the virus, and the use of a gene gun to shoot the virus into the cancer stem cells. See, at least, https://www.thermofisher.com/us/en/home/references/qibco-cell-culture-basics/transfection-basics/methods.html (downloaded Apr. 24, 2023), hereby, incorporated herein by reference in it's entirety.

### Amount of Virus and Protein Administered Using the Systems Taught Herein

Without being bound by any theory or mechanism of action, it should be appreciated that rate of infection, or multiplicity of infection (MOI) will be a factor in determining the amounts of oncolytic virus and protein expressed that is administered by the systems taught herein. The MOI is the number of viral particles that infect each cell and can range from 0.1 to 10, 20, 30, etc. By choosing an MOI for the cancer stem cells used, you can gain some control over the amount of virus/protein administered to a subject. The process includes selecting the cancer stem cells (e.g. using antibiotic resistance or fluorescence), determining relative transduction efficiency (functional titer) of the cancer stem cells for the oncolytic virus, determine the MOI of the cell line for the oncolytic virus. For example, assuming the functional titer of the viral particles is 1×10⁷ TU/ml, for 1×10⁷ cells, then 1 ml of the viral suspension is added to achieve an MOI=1. One of skill will appreciate that the MOI for pooled screening needs to be adjusted, perhaps using an MOI of 0.3 for best results. For example, assuming the functional titer of the viral particles is 1×10⁷ TU/ml, for 1×10⁷ cells, then 300 ul of the viral suspension is added to achieve an MOI=0.3. See, for example, https://www.transomic.com/cms/Transomic/media/Homepage/FAQ %20Guidelines/CRISPR/FAQ-Whv-calculate-the-MOI.pdf (provides an example protocol with state-of-art explanation; downloaded Apr. 24, 2023), hereby incorporated herein by reference in it's entirety.

In some embodiments, we can load glioblastoma cancer stem cells with an oncolytic herpes simplex virus and, if we infect the cancer stem cells at an MOI of 0.1, data shows we can expect a viral titer of about 3.8-4.0 log plaque forming unit per ml (log pfu/ml) after about 48 hours of infection for an oncolytic herpes simplex virus expressing IL-12 or no cytokine. See, at least, htts://www.ncbi.nlm.nih.gov/pmc/articles/PMC3718117/pdf/pnas.201307935.pdf (downloaded Apr. 24, 2023), hereby incorporated herein by reference in its entirety. IL-12 expressing virus began secreting substantial IL-12 at 24 hrs after infection and continued secretions for at least 96 hrs after infection, ranging from an amount of about 5 ng/ml at about 24 hrs after infection to an amount of about 60 ng/ml at about 96 hrs after infection.

It should be appreciated that each system designed will require some investigation into the replication kinetics of each combination of cancer stem cell and oncolytic virus chosen for the system. As the selection of cancer stem cells change, selection of oncolytic viruses change, and selection of desired proteins expressed change, the replication kinetics, and thus secretion amounts, can be expected to change. In addition, the programmed time-to-death of the reprogrammed cancer stem cells will also modify the amount of virus and desired protein administered by any given system to the subject treated.

These design features are known to those of skill and can be implemented without undue experimentation in the design of any of the systems taught herein. In view of these considerations, for example, a method of determining the amount of virus and the amount of desired protein released might include the following steps:
Selecting a desired cancer stem cell;
Selecting an amount of cancer stem cells to administer;
Selecting an oncolytic virus to load into the desired amount of cancer stem cells;
Selecting an MOI for the desired cancer stem cell, where MOI is defined as “the number of viral particles per cell being loaded”, an MOI of 0.1 means 1 viral particle for every 10 cancer stem cells, and an MOI of 10 means 10 viral particles for every 1 cancer stem cell; and,
Measure the viral titer per time after infection to determine log pfu/ml of the oncolytic virus;
Measure the amount of desired protein secreted into the supernatant per time after infection to determine amount of desired protein that will be administered to the subject in the secretions; and,
Measure the time to apoptosis of the cancer stem cells.

It should be appreciated that the measure of replication kinetics and amount of desired protein secreted is done to determine amounts of virus and protein made available “per dose” after administration. As such, since the number of doses will vary per treatment, the number of doses in a treatment regimen. In some embodiments, the cancer stem cells are administered in 1 dose, 2 doses, 3 doses, 4 doses, or 5 doses. The frequency of administration of doses, in some embodiments, can be 1 week apart, 2 weeks apart, 3 weeks apart, 4 weeks apart, or 5 weeks apart. The amount of antigen immune response delivered from the cancer stem cells alone can be adjusted by selecting an amount of reprogrammed cancer stem cells administered to the subject, and selecting a programmed time to death of the cancer stem cell. It has been determined that these conditions will provide a desired immune response in the subject. One of skill will appreciate that these features can be provided to the system using methods known to those of skill.

For the “double-killer” effect, the reprogrammed cancer stem cells are loaded with an oncolytic virus using methods taught herein, for the release of the virus from the reprogrammed cancer stem cells. The amount of oncolytic virus delivered to the subject from the cancer stem cells can range from 10 pfu to 10¹³ pfu or more. Alternatively, depending on the kind of virus and the titer attainable, delivery of 1 to 100 vp, 10 to 50 vp, 10 to 500 vp, 100-1000 vp, or up to about or at least about 1×10⁴ vp, 1×10⁵ vp, 1×10⁶ vp, 1×10⁷ vp, 1×10⁶ vp, 1×10⁹ vp, 1×10¹⁰ vp, 1×10¹¹ vp, 1×10¹² vp, 1×10¹³ vp, 1×10¹⁴ vp, 1×10¹⁵ vp, or 1×10¹⁶ vp or higher infectious viral particles (vp) can be used. The amount of antigen immune response delivered from the cancer stem cells alone can be adjusted by selecting an amount of reprogrammed cancer stem cells administered to the subject, and selecting a programmed time to death of the cancer stem cell. The amount of oncolytic virus delivered from the cancer stem cells can be adjusted by selecting an amount of virus loaded into the cancer stem cell, selecting an engineered rate of replication of the virus in the cancer stem cell, and selecting a programmed time to death of the cancer stem cell. One of skill will appreciate that these features can be provided to the system using methods known to those of skill.

For the “triple-killer” effect, the reprogrammed cancer stem cells are loaded with an oncolytic virus using methods taught herein, and the oncolytic virus is designed to express one or more desired proteins as taught herein. The amount of antigen immune response provides the first killer effect, the release of the oncolytic virus provides the second killer effect, and the expression of the one or more desired proteins from the oncolytic virus provides the third killer effect. The amount of antigen immune response delivered from the cancer stem cells alone can be adjusted by selecting an amount of reprogrammed cancer stem cells administered to the subject, and selecting a programmed time to death of the cancer stem cell. The amount of oncolytic virus delivered from the cancer stem cells can be adjusted by selecting an amount of virus loaded into the cancer stem cell, selecting an engineered rate of replication of the virus in the cancer stem cell, selecting a programmed time to death of the cancer stem cell, and selecting a targeted amount of the desired protein to deliver to the subject from the oncolytic virus. One of skill will appreciate that these features can be provided to the system using methods known to those of skill.

As such, a method of creating a system that expresses a desired protein can including measuring the amount of the desired protein expressed by the oncolytic virus in the cancer stem cell. The measuring can include determining the rate of production of the desired protein expressed. Those of skill will appreciate that the amount of protein translation can be measured using any of a variety of techniques known in the art. For example, the skilled artisan could test the system using any method selected from the group consisting of “RNA-SEQ”, “TRAP-SEQ” (translating ribosome affinity purification-sequencing), and “Proximity-specific ribosome profiling”, “p-SILAC” (pulsed Stable Isotope Labeling of Amino acids in Culture), “BONCAT” (bio-orthogonal non-canonical amino acid tagging), “QUANCAT” (quantitative non-canonical amino acid tagging), “HILAQ” (Heavy Isotope Labeled Azidohomoalanine Quantification), and labeling of nascent proteins with the antibiotic puromycin (e.g., “PUNCH-P” with biotinylated puromycin). See, for example, Dermit, M. et al. Mol. BioSyst. 13(12):2477-2488(2017)(a review of the state-of-the-art of measuring protein translation).

1. A method of treating a glioblastoma in a subject, comprising:
obtaining a protective, antigenic drug delivery system comprising reprogrammed glioblastoma stem cells hosting an oncolytic virus specific to the glioblastoma stem cells; wherein, the delivery system is configured for
protecting the oncolytic virus during a transport of the oncolytic virus in the subject to increase the half-life of the oncolytic virus during the transport when compared to an unprotected transport of the oncolytic virus in the subject;
amplifying the amount of the oncolytic virus during the protected transport of the oncolytic virus in the subject; and,
killing the glioblastoma stem cells in a desired amount of time into the transport to (i) release the oncolytic virus in the subject from the dead glioblastoma stem cells; (ii) initiate an antigenic immune response in the subject to antigens released from the dead glioblastoma stem cells; and, (iii) continue replication of the virus in tumor cells of the cancer.
and,
administering the antigenic delivery system to the subject.

2. The method of claim 1, wherein the obtaining of the delivery system includes
creating the protective, antigenic delivery system, the creating including
obtaining the glioblastoma stem cells;
reprogramming the glioblastoma stem cells to die in a desired time for a programmed release of the oncolytic virus and the antigens from the glioblastoma stem cell in the subject;
obtaining the oncolytic virus; and,
loading the glioblastoma stem cells with the oncolytic virus to create the protective, antigenic delivery system.

3. The method of claim 2, wherein the obtaining of the glioblastoma stem cells includes harvesting the glioblastoma stem cells from the subject.

4. The method of claim 2, wherein the reprogramming of the glioblastoma stem cells to die in the desired time for the programmed release of the oncolytic virus and the antigens from the glioblastoma stem cell in the subject includes exposing the glioblastoma stem cells to radiation.

5. The method of claim 2, wherein the reprogramming of the glioblastoma stem cells to die in the desired time for the programmed release of the oncolytic virus and the antigens from the glioblastoma stem cell in the subject includes selecting the type of the oncolytic virus, the amount of the oncolytic virus, or a combination thereof, used in the loading of the glioblastoma stem cells with the oncolytic virus.

6. The method of claim 2, wherein the obtaining of the oncolytic virus includes
creating the oncolytic virus from a wild-type virus selected from the group consisting of Herpes Simplex Virus (HSV), Newcastle Disease Virus (NDV), Vesicular Stomatitis Virus (VSV), Reovirus, Measles Virus, Retrovirus, Influenza Virus, Sindbis Virus, Vaccinia Virus, and Adenovirus.

7. The method of claim 2, wherein the obtaining of the oncolytic virus includes
creating the oncolytic virus from a wild-type virus selected from the group consisting of Herpes Simplex Virus (HSV), Newcastle Disease Virus (NDV), Vesicular Stomatitis Virus (VSV), Reovirus, Measles Virus, Retrovirus, Influenza Virus, Sindbis Virus, Vaccinia Virus, and Adenovirus;
and, the creating includes
deleting undesired genes from the wild-type oncolytic virus to make the virus tumor-specific; and,
inserting desired genes that express desired proteins from the oncolytic virus.

8. The method of claim 7, wherein the desired proteins are immunomodulators.

9. The method of claim 7, wherein the desired proteins are cytokines.

10. The method of claim 7, wherein the desired proteins are chemokines.

11. A protective, antigenic drug delivery system, comprising:
reprogrammed cancer stem cells; and,
an oncolytic virus specific to the cancer stem cells;
wherein, the delivery system is configured for
protecting the oncolytic virus during a transport of the oncolytic virus in the subject to increase the half-life of the oncolytic virus during the transport when compared to an unprotected transport of the oncolytic virus in the subject;
amplifying the amount of the oncolytic virus during the protected transport of the oncolytic virus in the subject; and,
killing the cancer stem cells in a desired amount of time into the transport to (i) release the oncolytic virus in the subject from the dead cancer stem cells; (ii) initiate an antigenic immune response in the subject to antigens released from the dead cancer stem cells; and, (iii) continue replication of the virus in tumor cells of a cancer.

12. The protective, antigenic drug delivery system of claim 11, the delivery system is configured using a process that includes
obtaining the cancer stem cells;
reprogramming the cancer stem cells to die in a desired time for a programmed release of the oncolytic virus and the antigens from the cancer stem cell in the subject;
obtaining the oncolytic virus; and,
loading the cancer stem cells with the oncolytic virus to create the protective, antigenic delivery system.

13. The protective, antigenic drug delivery system of claim 12, wherein the obtaining of the cancer stem cells includes harvesting the cancer stem cells from the subject.

14. The protective, antigenic drug delivery system of claim 12, wherein the reprogramming of the cancer stem cells to die in the desired time for the programmed release of the oncolytic virus and the antigens from the cancer stem cell in the subject includes exposing the cancer stem cells to radiation.

15. The protective, antigenic drug delivery system of claim 12, wherein the reprogramming of the cancer stem cells to die in the desired time for the programmed release of the oncolytic virus and the antigens from the cancer stem cell in the subject includes selecting the type of the oncolytic virus, the amount of the oncolytic virus, or a combination thereof, used in the loading of the cancer stem cells with the oncolytic virus.

16. The protective, antigenic drug delivery system of claim 12, wherein the obtaining of the oncolytic virus includes
creating the oncolytic virus from a wild-type virus selected from the group consisting of Herpes Simplex Virus (HSV), Newcastle Disease Virus (NDV), Vesicular Stomatitis Virus (VSV), Reovirus, Measles Virus, Retrovirus, Influenza Virus, Sindbis Virus, Vaccinia Virus, and Adenovirus.

17. The protective, antigenic drug delivery system of claim 12, wherein the obtaining of the oncolytic virus includes
creating the oncolytic virus from a wild-type virus selected from the group consisting of Herpes Simplex Virus (HSV), Newcastle Disease Virus (NDV), Vesicular Stomatitis Virus (VSV), Reovirus, Measles Virus, Retrovirus, Influenza Virus, Sindbis Virus, Vaccinia Virus, and Adenovirus;
and, the creating includes
deleting undesired genes from the wild-type oncolytic virus to make the virus tumor specific; and,
inserting desired genes that express desired proteins from the oncolytic virus.

18. The protective, antigenic drug delivery system of claim 17, wherein the desired proteins are immunomodulators.

19. The protective, antigenic drug delivery system of claim 17, wherein the desired proteins are cytokines.

20. The protective, antigenic drug delivery system of claim 17, wherein the desired proteins are chemokines.


SEQUENCE LISTING (128 sequences)

SEQ ID NO: 1 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 2 - protein, 189 aa, Mus musculus
MARLCAFLMVLAVMSYWPTCSLGCDLPQTHNLRNKRALTLLVQMRRLSPLSCLKDRKDFGFPQEKVDAQQIKKAQAIPVLSELTQQILNIFTSKDSSAAWNATLLDSFCNDLHQQLNDLQGCLMQQVGVQEFPLTQEDALLAVRKYFHRITVYLREKKHSPCAWEVVRAEVWRALSSSANVLGRLREEK

SEQ ID NO: 3 - DNA, 570 bp, Homo sapiens
  source 1..570: mol_type = genomic DNA
atggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataa

SEQ ID NO: 4 - DNA, 570 bp, Mus musculus
  source 1..570: mol_type = genomic DNA
atggctaggctctgtgctttcctgatggtcctggcggtgatgagctactggccaacctgctctctaggatgtgacctgcctcagactcataacctcaggaacaagagagccttgacactcctggtacaaatgaggagactctcccctctctcctgcctgaaggacaggaaggactttggattcccgcaggagaaggtggatgcccagcagatcaagaaggctcaagccatccctgtcctgagtgagctgacccagcagatcctgaacatcttcacatcaaaggactcatctgctgcttggaatgcaaccctcctagactcattctgcaatgacctccaccagcagctcaatgacctgcaaggttgtctgatgcagcaggtgggggtgcaggaatttcccctgacccaggaagatgccctgctggctgtgaggaaatacttccacaggatcactgtgtacctgagagagaagaaacacagcccctgtgcctgggaggtggtcagagcagaagtctggagagccctgtcttcctctgccaatgtgctgggaagactgagagaagagaaatga

SEQ ID NO: 5 - protein, 133 aa, Homo sapiens [sequence omitted]

SEQ ID NO: 6 - protein, 169 aa, Mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTESKSFQLEDAENFISNIRVTVVKLKGSDNTFECQFDDESATVVDFLRRWIAFCQSIISTSPQ

SEQ ID NO: 7 - DNA, 402 bp, Homo sapiens
  source 1..402: mol_type = genomic DNA
atgcctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctgaaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcaaagcatcatctcaacactgacttga

SEQ ID NO: 8 - DNA, 510 bp, Mus musculus
  source 1..510: mol_type = genomic DNA
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactgaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataa

SEQ ID NO: 9 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 11 - protein, 215 aa, Mus musculus
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 12 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 13 - DNA, 762 bp, Homo sapiens
  source 1..762: mol_type = genomic DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 14 - DNA, 987 bp, Homo sapiens
  source 1..987: mol_type = genomic DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 15 - DNA, 648 bp, Mus musculus
  source 1..648: mol_type = genomic DNA
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 16 - DNA, 1008 bp, Mus musculus
  source 1..1008: mol_type = genomic DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatcctag

SEQ ID NO: 17 - protein, 135 aa, Homo sapiens
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 18 - protein, 162 aa, Mus musculus
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 19 - DNA, 408 bp, Homo sapiens
  source 1..408: mol_type = genomic DNA
atggtattgggaaccatagatttgtgcagctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 20 - DNA, 489 bp, Mus musculus
  source 1..489: mol_type = genomic DNA
atgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctga

SEQ ID NO: 21 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 22 - protein, 192 aa, Mus musculus
MAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 23 - DNA, 582 bp, Homo sapiens
  source 1..582: mol_type = genomic DNA
atggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 24 - DNA, 579 bp, Mus musculus
  source 1..579: mol_type = genomic DNA
atggctgccatgtcagaagactcttgcgtcaacttcaaggaaatgatgtttattgacaacacgctttactttatacctgaagaaaatggagacctggaatcagacaactttggccgacttcactgtacaaccgcagtaatacggaatataaatgaccaagttctcttcgttgacaaaagacagcctgtgttcgaggatatgactgatattgatcaaagtgccagtgaaccccagaccagactgataatatacatgtacaaagacagtgaagtaagaggactggctgtgaccctctcggtgaaggatagtaaaatgtctaccctctcctgtaagaacaagatcatttcctttgaggaaatggatccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagttag

SEQ ID NO: 25 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 26 - protein, 146 aa, Mus musculus [sequence omitted]

SEQ ID NO: 27 - DNA, 489 bp, Homo sapiens
  source 1..489: mol_type = genomic DNA
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

SEQ ID NO: 28 - DNA, 441 bp, Mus musculus
  source 1..441: mol_type = genomic DNA
atggagaggacccttgtctgtctggtagtcatcttcttggggacagtggcccataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaagatgattcatcagcatctctcctag

SEQ ID NO: 29 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 30 - protein, 190 aa, Mus musculus
MARLCAFLVMLIVMSYWSTCSLGCDLPHTYNLRNKRALKVLAQMRRLPFLSCLKDRQDFGFPLEKVDNQQIQKAQAIPVLRDLTQQTLNLFTSKASSAAWNTTLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 31 - DNA, 567 bp, Homo sapiens
  source 1..567: mol_type = genomic DNA
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 32 - DNA, 573 bp, Mus musculus
  source 1..573: mol_type = genomic DNA
atggctagactctgtgctttcctcgtgatgctgatagtgatgagctactggtcaacctgttctctaggatgcgatctgcctcacacttataacctcaggaacaagagggccttgaaggtcctggcacagatgaggaggctcccctttctctcctgcctgaaggacaggcaggactttggattccccctggagaaggtggataaccagcagatccagaaggctcaagccatccctgtgctgcgagatcttactcagcagaccttgaacctcttcacatcaaaggcttcatctgctgcttggaatacaaccctcctagactcattctgcaatgacctccaccagcagctcaatgacctgcaaacctgtctgatgcagcaggtgggggtgcaggaacctcctctgacccaggaagacgccctgctggctgtgaggaaatatttccacaggatcactgtgtacctgagagagaagaaacacagcccctgtgcctgggaggtggtcagagcagaagtctggagagccctgtcttcctcagtcaacttgctgccaagactgagtgaagagaaggagtga

SEQ ID NO: 33 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 34 - protein, 155 aa, Mus musculus
MNATHCILALQLFLMAVSGCYCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 35 - DNA, 501 bp, Homo sapiens
  source 1..501: mol_type = genomic DNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 36 - DNA, 468 bp, Homo sapiens
  source 1..468: mol_type = genomic DNA
atgaacgctacacactgcatcttggctttgcagctcttcctcatggctgtttctggctgttactgccacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgctga

SEQ ID NO: 37 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 38 - protein, 235 aa, Mus musculus
MSTESMIRDVELAEEALPQKMGGFQNSRRCLCLSLFSFLLVAGATTLFCLLNFGVIGPQRDEKFPNGLPLISSMAQTLTLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 39 - DNA, 702 bp, Homo sapiens
  source 1..702: mol_type = genomic DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 40 - DNA, 708 bp, Mus musculus
  source 1..708: mol_type = genomic DNA
atgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgagaagttcccaaatggcctccctctcatcagttctatggcccagaccctcacactcagatcatcttctcaaaattcgagtgacaagcctgtagcccacgtcgtagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctgtga

SEQ ID NO: 41 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 42 - protein, 169 aa, Mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 43 - DNA, 462 bp, Homo sapiens
  source 1..462: mol_type = genomic DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 44 - DNA, 510 bp, Mus musculus
  source 1..510: mol_type = genomic DNA
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataa

SEQ ID NO: 45 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 46 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 47 - DNA, 435 bp, Homo sapiens
  source 1..435: mol_type = genomic DNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 48 - DNA, 426 bp, Mus musculus
  source 1..426: mol_type = genomic DNA
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaatga

SEQ ID NO: 49 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 50 - protein, 113 aa, Mus musculus
MPQEDIHFGYPRKGHRRSYVTAEEAAGIGILIVVLGIALLIGCWYCRRRSGYRTLMDKRRHIGIQKTSRERCSCESPDHQDSRLSSQEKSHQPVVPNAPPAYEKLSSPPPYSP

SEQ ID NO: 51 - DNA, 357 bp, Homo sapiens
  source 1..357: mol_type = genomic DNA
atgccaagagaagatgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaa

SEQ ID NO: 52 - DNA, 342 bp, Mus musculus
  source 1..342: mol_type = genomic DNA
atgccccaagaagacattcactttggttatcccaggaaggggcacagacgctcctatgtcactgctgaagaggccgcagggatcggcatcctgatcgtggtcctggggattgctctgcttatcggctgctggtactgtagaagacgaagtggatacagaaccttgatggacaaaaggcgtcatattggtattcaaaaaacctcaagggaaagatgctcatgtgagagccctgatcaccaggacagccgactgtcttctcaagagaaatcccatcagcccgtggttcccaacgctccgcctgcctatgagaagctctcttcaccgccaccttattcaccctga

SEQ ID NO: 53 - protein, 193 aa, Homo sapiens; identical to SEQ ID NO: 21

SEQ ID NO: 54 - DNA, 582 bp, Homo sapiens; identical to SEQ ID NO: 23
  source 1..582: mol_type = genomic DNA

SEQ ID NO: 55 - protein, 134 aa, Homo sapiens
MQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 56 - skipped

SEQ ID NO: 57 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 58 - DNA, 426 bp, Mus musculus
  source 1..426: mol_type = genomic DNA
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaatga

SEQ ID NO: 59 - DNA, 426 bp, Mus musculus
  source 1..426: mol_type = genomic DNA
atgtggctccagaacctcctcttcctcggtatcgtcgtgtattcactctccgcacctactcgctcacctatcactgtcaccagaccctggaagcacgtggaggccatcaaggaggctctgaacctgctggacgatatgccagtgaccctgaacgaggaggtggaggtggtgagcaacgagttctcctttaagaagctgacctgcgtgcagacaaggctgaagatcttcgagcagggcctgagaggaaactttaccaagctgaagggcgccctgaacatgaccgcttcttactaccagacatactgcccccctacccccgagacagactgtgagacacaggtgaccacatacgccgacttcattgatagcctgaaaacattcctgaccgacattccatttgagtgtaagaagcccgtccagaagtaa

SEQ ID NO: 60 - protein, 144 aa, Homo sapiens; identical to SEQ ID NO: 45

SEQ ID NO: 61 - DNA, 435 bp, Homo sapiens; identical to SEQ ID NO: 47
  source 1..435: mol_type = genomic DNA

SEQ ID NO: 62 - DNA, 435 bp, Homo sapiens
  source 1..435: mol_type = genomic DNA
atgtggctgcagtccctgctgctgctgggcaccgtcgcctgttctatttccgcacccgcaaggtcaccaagtccatctactcagccttgggagcacgtgaacgcaatccaggaggcacggcggctgctgaacctgagccgggacaccgccgccgagatgaacgagacagtggaagtgatcagcgagatgttcgatctgcaggagcccacctgcctgcagacaaggctggagctgtacaagcagggcctgcgcggctctctgaccaagctgaagggcccactgacaatgatggccagccactataagcagcactgcccccctacccccgagacaagctgtgccacccagatcatcacattcgagtcctttaaggagaacctgaaggattttctgctggtcattccatttgattgttgggagcccgtccaggagtaa

SEQ ID NO: 63 - protein, 669 aa, Gibbon ape leukemia virus
MVLLPGSMLLTSNLHHLRHQMSPGSWKRLIILLSCVFGGGGTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKI

SEQ ID NO: 64 - DNA, 2010 bp, Gibbon ape leukemia virus
  source 1..2010: mol_type = genomic DNA
atggtattgctgcctgggtccatgcttctcacctcaaacctgcaccaccttcggcaccagatgagtcctgggagctggaaaagactgatcatcctcttaagctgcgtattcggcggcggcgggacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaataagttagttcaattcatcaatgataggataagtgcagttaaaatttaa

SEQ ID NO: 65 - DNA, 2013 bp, Gibbon ape leukemia virus
  source 1..2013: mol_type = genomic DNA
accatggtcctgctgcctgggtctatgctgctgacttctaacctgcaccacctgcgacaccagatgtctcccggctcatggaaacggctgatcatcctgctgagctgcgtgttcggaggaggaggcacctccctgcagaacaagaatcctcaccagccaatgaccctgacatggcaggtgctgtcccagacaggcgacgtggtgtgggataccaaggcagtgcagccaccttggacatggtggcccaccctgaagcctgacgtgtgcgccctggccgcctccctggagtcttgggacatccccggcacagacgtgagcagcagcaagagggtgagaccacccgactctgattatacagccgcctacaagcagatcacctggggcgccatcggctgtagctatcctcgggcccgcacaaggatggccagctccaccttttacgtgtgcccacgcgacggaaggaccctgtctgaggcaaggagatgtggcggcctggagagcctgtattgcaaggagtgggattgtgagaccacaggcacaggctactggctgtctaagtctagcaaggacctgatcaccgtgaagtgggatcagaacagcgagtggacacagaagttccagcagtgccaccagaccggctggtgtaatcccctgaagatcgactttacagataagggcaagctgtccaaggactggatcaccggcaagacatggggcctgagattctacgtgtctggccaccctggcgtgcagtttacaatccggctgaagatcaccaacatgccagcagtggcagtgggaccagacctggtgctggtggagcagggacctccacgcacctccctggccctgccccctccactgccccctagggaggccccaccccctagcctgcccgattctaacagcacagccctggccacctccgcccagacccctacagtgcgcaagaccatcgtgacactgaataccccaccccctaccacaggcgacaggctgttcgatctggtgcagggcgcctttctgacactgaacgccaccaatcctggcgcaaccgagagctgctggctgtgcctggctatgggcccaccctactatgaggcaatcgcctcctctggagaggtggcatattccacagacctggatagatgcagatggggcacccagggcaagctgaccctgacagaggtgtctggccacggcctgtgcatcggcaaggtgccattcacacaccagcacctgtgcaaccagaccctgagcatcaatagctccggcgaccaccagtacctgctgccaagcaaccactcctggtgggcatgctccacaggactgaccccatgtctgagcaccagcgtgttcaaccagaccagagacttttgtatccaggtgcagctgatccctcggatctactattacccagaggaggtgctgctgcaggcctatgataattcccacccaagaacaaagagggaggccgtgtctctgaccctggccgtgctgctgggactgggaatcacagcaggaatcggcacaggcagcaccgccctgatcaagggaccaatcgacctgcagcagggactgacctccctgcagatcgccatcgacgccgatctgagagccctgcaggacagcgtgtccaagctggaggattctctgacctctctgagcgaggtggtgctgcagaacaggaggggcctggacctgctgttcctgaaggagggaggactgtgcgccgccctgaaggaggagtgctgtttttatatcgaccactctggcgccgtgcgggatagcatgaagaagctgaaggagaagctggataagcgccagctggagaggcagaagagccagaattggtacgagggctggttcaacaattccccctggtttaccacactgctgtctaccatcgcaggacctctgttattactgctgctgctgctgatcctgggcccatgtatcatcaacaagctggtgcagtttatcaacgaccgaatctccgcagtgaaaatctaa

SEQ ID NO: 66 - protein, 252 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSIHLGGVFELQPGASVFVNVTDPSQVSHGTGFTSFGLLKL

SEQ ID NO: 67 - DNA, 759 bp, synthetic construct
  source 1..759: mol_type = other DNA
atgatcgagacctacaatcagacaagcccacggtccgccgcaaccggactgcctatcagcatgaagatcttcatgtacctgctgaccgtgtttctgatcacacagatgatcggctccgccctgttcgccgtgtatctgcacaggagactggacaagatcgaggatgagcgcaatctgcacgaggacttcgtgtttatgaagaccatccagcggtgcaacacaggcgagaggagcctgtctctgctgaattgtgaggagatcaagtcccagttcgagggctttgtgaaggatatcatgctgaacaaggaggagacaaagaaggacgaggatccacagatcgcagcacacgtggtgtccgaggcaaactctaatgccgccagcgtgctgcagtgggccaagaagggctactataccatgaagtctaacctggtgacactggagaatggcaagcagctgaccgtgaagaggcagggcctgtactatatctatgcccaggtgacattctgctctaacagagaggcaagctcccaggcacccttcatcgtgggactgtggctgaagccctctagcggcagcgagaggatcctgctgaaggccgccaatacccactcctctagccagctgtgcgagcagcagtccatccacctgggaggcgtgttcgagctgcagcctggagccagcgtgttcgtgaacgtgacagacccatctcaggtgagccacggcaccggcttcacaagctttggcctgctgaagctgtga

SEQ ID NO: 68 - protein, 471 aa, Homo sapiens
MLPFLSMLVLLVQPLGNLGAEMKSLSQRSVPNTCTLVMCSPTENGLPGRDGRDGREGPRGEKGDPGLPGPMGLSGLQGPTGPVGPKGENGSAGEPGPKGERGLSGPPGLPGIPGPAGKEGPSGKQGNIGPQGKPGPKGEAGPKGEVGAPGMQGSTGAKGSTGPKGERGAPGVQGAPGNAGAAGPAGPAGPQGAPGSRGPPGLKGDRGVPGDRGIKGESGLPDSAALRQQMEALKGKLQRLEVAFSHYQKAALFPDGHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 69 - DNA, 1416 bp, Homo sapiens
  source 1..1416: mol_type = genomic DNA
atgctgccctttctgagcatgctggtgctgctggtgcagcctctgggaaacctgggagccgagatgaagagcctgtcccagagatctgtgcctaacacctgcacactggtcatgtgcagccccaccgagaatggactgcctggaagggacggaagggatggaagggagggccctcggggcgagaagggcgacccaggactgcctggaccaatgggactgagcggactgcagggaccaacaggacctgtgggaccaaagggagagaacggatccgccggagagccaggccctaagggcgagaggggcctgtctggcccccctggcctgccaggcatcccaggccccgccggcaaggagggcccatccggcaagcagggcaatatcggcccccagggcaagcctggcccaaagggcgaggcaggaccaaagggagaagtgggagcacctggcatgcagggatccaccggagcaaagggatctacaggaccaaagggcgagcgcggcgccccaggcgtgcagggcgcccccggcaatgcaggagcagcaggaccagcaggacctgcaggcccacagggcgcccctggctctaggggcccacccggcctgaagggcgacaggggagtgcctggcgataggggcatcaagggagagagcggactgccagattccgccgccctgaggcagcagatggaggccctgaagggcaagctgcagaggctggaggtggccttctcccactaccagaaggccgccctgtttccagacggccacaggagactggacaagatcgaggatgagcgcaacctgcacgaggatttcgtgtttatgaagaccatccagagatgcaacacaggcgagcggtctctgagcctgctgaattgtgaggagatcaagtctcagttcgagggctttgtgaaggacatcatgctgaacaaggaggagaccaagaaggagaatagcttcgagatgcagaagggcgatcagaatccccagatcgcagcacacgtgatcagcgaggcaagctccaagaccacatccgtgctgcagtgggccgagaagggctactataccatgtccaacaatctggtgacactggagaacggcaagcagctgaccgtgaagagacagggcctgtactatatctatgcccaggtgacattctgctctaatcgggaggcctctagccaggccccttttatcgcctctctgtgcctgaagagcccaggcagattcgagcggatcctgctgagggccgccaacacccactcctctgccaagccatgcggacagcagagcatccacctgggaggcgtgttcgagctgcagccaggagcctccgtgtttgtgaatgtgacagacccatcccaggtgtctcacggaaccggcttcacatcctttggcctgctgaagctgtga

SEQ ID NO: 70 - protein, 470 aa, Mus musculus
MLPFLSMLVLLVQPLGNLGAEMKSLSQRSVPNTCTLVMCSPTENGLPGRDGRDGREGPRGEKGDPGLPGPMGLSGLQGPTGPVGPKGENGSAGEPGPKGERGLSGPPGLPGIPGPAGKEGPSGKQGNIGPQGKPGPKGEAGPKGEVGAPGMQGSTGAKGSTGPKGERGAPGVQGAPGNAGAAGPAGPAGPQGAPGSRGPPGLKGDRGVPGDRGIKGESGLPDSAALRQQMEALKGKLQRLEVAFSHYQKAALFPDGHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSIGSERILLKAANTHSSSQLCEQQSVHLGGVFELQAGASVFVNVTEASQVIHRVGFSSFGLLKL

SEQ ID NO: 71 - DNA, 1412 bp, Mus musculus
  source 1..1412: mol_type = genomic DNA
atgctgcccttcctgagcatgctggtgctgctggtgcagcctctgggcaatctgggcgccgagatgaagtccctgtctcagaggagcgtgccaaacacctgcacactggtcatgtgctctccaaccgagaatggactgccaggaagggacggaagagatggaagggagggaccaaggggagagaagggcgaccctggactgcctggaccaatgggactgtccggactgcagggaccaacaggccctgtgggaccaaagggagagaatggaagcgccggagagccaggacctaagggagagaggggcctgtccggcccccctggcctgcctggcatcccaggccccgccggcaaggagggcccttctggcaagcagggcaacatcggaccacagggcaagcctggaccaaagggagaggcaggaccaaagggagaagtgggagcacccggcatgcagggcagcaccggagcaaagggatccaccggccctaagggagagagaggagcacctggagtgcagggcgccccaggcaatgcaggagcagcaggaccagcaggacctgcaggcccacagggcgccccaggcagccggggcccacccggcctgaagggcgacaggggagtgccaggcgataggggcatcaagggagagtccggactgccagactctgccgccctgaggcagcagatggaggccctgaagggcaagctgcagaggctggaggtggccttctcccactaccagaaggccgccctgtttccagacggacacaggagactggataaggtggaggaggaggtgaacctgcacgaggatttcgtgttcatcaagaagctgaagaggtgcaacaagggcgagggcagcctgtccctgctgaattgtgaggagatgcggcgccagttcgaggacctggtgaaggatatcaccctgaacaaggaggagaagaaggagaattcttttgagatgcagaggggcgacgaggatcctcagatcgcagcacacgtggtgtccgaggcaaactctaatgccgccagcgtgctgcagtgggccaagaagggctactataccatgaagtctaacctggtcatgctggagaatggcaagcagctgacagtgaagagagagggcctgtactacgtgtacacccaggtgacattctgcagcaacagagagcccagctcccagcggccttttatcgtgggcctgtggctgaagccctctatcggaagcgagaggatcctgctgaaggcagccaatacccactctagctcccagctgtgcgagcagcagtccgtgcacctgggaggcgtgttcgagctgcaggcaggagcaagcgtgttcgtgaacggacagaggccagccaggtcatccacagagtgggcttctctagctttggcctgctgaagctgtga

SEQ ID NO: 72 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 73 - DNA, 786 bp, Homo sapiens
  source 1..786: mol_type = genomic DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 74 - protein, 260 aa, Mus musculus
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 75 - DNA, 783 bp, Mus musculus
  source 1..783: mol_type = genomic DNA
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctga

SEQ ID NO: 76 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 77 - DNA, 765 bp, Homo sapiens
  source 1..765: mol_type = genomic DNA
atggagtacgcctctgacgccagcctggatccagaggccccttggccacctgcaccaagggcccgcgcctgccgcgtgctgccctgggccctggtggccggcctgttattactgctgctgctggccgccgcctgcgccgtgttcctggcatgtccttgggccgtgagcggagccagagcctccccaggctctgccgccagccctcggctgagagagggaccagagctgtccccagacgatccagcaggcctgctggacctgaggcagggaatgtttgcccagctggtggcccagaacgtgctgctgatcgacggccccctgtcctggtactctgatcctggcctggccggcgtgtctctgaccggcggcctgagctataaggaggatacaaaggagctggtggtggccaaggccggcgtgtactacgtgttcttccagctggagctgaggagagtggtggcaggagagggctctggaagcgtgtccctggccctgcacctgcagcccctgcggagcgccgcaggagccgccgccctggccctgaccgtggacctgccaccagccagctccgaggcaaggaattccgccttcggctttcagggcagactgctgcacctgtctgccggacagaggctgggagtgcacctgcacaccgaggccagggcccgccacgcatggcagctgacccagggagcaacagtgctgggcctgttccgcgtgacacctgagatcccagcaggcctgcctagcccacggtccgagtga

SEQ ID NO: 78 - skipped

SEQ ID NO: 79 - DNA, 1389 bp, Homo sapiens
  source 1..1389: mol_type = genomic DNA
atgctgctgttcctgctgtccgccctggtgctgctgacccagcctctgggctacctggaggccgagatgaagacctattctcaccggacaatgccaagcgcctgcacactggtcatgtgcagcagcgtggagtctggcctgccaggaagggacggaagggatggaagggagggacctagaggcgagaagggcgaccctggcctgccaggagcagcaggacaggcaggaatgcccggccaggccggccccgtgggacctaagggcgacaacggaagcgtgggagagccaggaccaaagggcgataccggcccttccggaccacctggaccaccaggcgtgcctggcccagccggcagggagggccctctgggcaagcagggcaatatcggcccacagggcaagcccggccctaagggcgaggccggccccaagggcgaagtgggcgcccctggcatgcagggaagcgccggagcccgcggcctggccggacctaagggcgagagaggcgtgcctggagagaggggcgtgccaggaaacacaggcgcagcaggatctgccggagcaatgggaccccagggcagccctggcgccaggggccctccaggcctgaagggcgacaagggcatcccaggcgataagggagcaaagggagagagcggcctgccagatgtggcctccctgcgccagcaggtggaggccctgcagggccaggtgcagcacctgcaggccgccttctctcagtacaagaaggtggagctgtttccaaacggcgcctgcccctgggccgtgagcggagcccgggcctccccaggctctgccgccagccctaggctgcgcgagggaccagagctgagcccagacgatccagcaggcctgctggacctgagacagggaatgttcgcccagctggtggcccagaatgtgctgctgatcgacggcccactgtcctggtactctgatccaggcctggccggcgtgtccctgaccggcggcctgtcttataaggaggatacaaaggagctggtggtggccaaggccggcgtgtactacgtgttcttccagctggagctgaggagagtggtggcaggagagggatccggatctgtgagcctggccctgcacctgcagcccctgcggtccgccgcaggagccgccgccctggccctgaccgtggacctgccacctgcctctagcgaggcacgcaattccgccttcggctttcagggccggctgctgcacctgtctgccggacagagactgggagtgcacctgcacaccgaggcccgggccagacacgcctggcagctgacccagggagcaacagtgctgggcctgtttagggtgacacctgagatcccagccggcctgccaagcccccgctccgagtga

SEQ ID NO: 80 - protein, 309 aa, Mus musculus
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 81 - DNA, 930 bp, Mus musculus
  source 1..930: mol_type = genomic DNA
atggatcagcacacactggacgtggaggataccgctgacgctaggcacccagctggcacctcctgcccttctgatgccgctctgctgcgcgacacaggactgctggccgatgccgctctgctgtctgacacagtgcggccaaccaacgccgctctgccaaccgatgctgcttaccctgctgtgaacgtgagggacagagaggctgcttggccacctgccctgaacttctgcagccgccaccctaagctgtacggcctggtggccctggtgctgctgctgctgatcgctgcttgcgtgccaatctttacccggacagagccacgccccgctctgacaatcaccacatcccccaacctgggcaccagggagaacaacgccgatcaggtgacaccagtgtctcacatcggctgccccaacaccacacagcagggaagcccagtgttcgccaagctgctggctaagaaccaggccagcctgtgcaacaccacactgaactggcacagccaggacggagctggaagctcctacctgtcccagggcctgagatacgaggaggataagaaggagctggtggtggactcccctggactgtactacgtgttcctggagctgaagctgtctccaacctttacaaacaccggccacaaggtgcagggatgggtgtctctggtgctgcaggctaagccccaggtggacgatttcgataacctggccctgaccgtggagctgtttccttgtagcatggagaacaagctggtggacaggtcttggagccagctgctgctgctgaaggctggccacaggctgtccgtgggactgagagcctacctgcacggcgcccaggatgcttacagagactgggagctgagctaccctaacaccacatccttcggactgtttctggtgaagcctgacaacccatgggagtga

SEQ ID NO: 82 - skipped

SEQ ID NO: 83 - DNA, 1389 bp, Mus musculus
  source 1..1389: mol_type = genomic DNA
atgctgcctttcctgtccatgctggtgctgctggtgcagccactgggcaacctgggagccgagatgaagtctctgagccagcgcagcgtgcctaacacctgcacactggtcatgtgctcccctacagagaacggcctgccaggaagggacggaagagatggaagggagggaccaaggggagagaagggcgaccccggactgcctggaccaatgggactgagcggcctgcagggaccaaccggccccgtgggacctaagggagagaacggatccgctggagagccaggacctaagggagagagaggactgtctggaccacctggactgccaggaatcccaggaccagctggcaaggagggaccatccggcaagcagggaaacatcggaccacagggaaagcctggaccaaagggagaggctggacctaagggagaagtgggcgccccaggaatgcagggctctacaggagctaagggcagcaccggaccaaagggagagaggggagcccccggagtgcagggagcccctggcaacgctggagccgctggcccagccggacccgctggccctcagggagcccccggctctaggggaccaccaggcctgaagggagacagaggcgtgcccggagatcggggcatcaagggagagagcggcctgcctgactccgccgctctgagacagcagatggaggctctgaagggcaagctgcagcggctggaggtggccttctcccactaccagaaggccgctctgtttcctgacggaaggacagagcccaggcctgctctgaccatcaccacatctccaaacctgggcacaagagagaacaacgccgatcaggtgacccccgtgtctcacatcggatgccctaacaccacacagcagggcagccccgtgtttgccaagctgctggctaagaaccaggccagcctgtgcaacaccacactgaactggcactcccaggatggcgccggaagctcctacctgtctcagggcctgcggtacgaggaggacaagaaggagctggtggtggatagcccaggcctgtactacgtgttcctggagctgaagctgtcccccacctttacaaacaccggacacaaggtgcagggatgggtgagcctggtgctgcaggctaagccccaggtggacgatttcgacaacctggccctgaccgtggagctgtttccttgctctatggagaacaagctggtggatagatcctggagccagctgctgctgctgaaggctggacaccgcctgagcgtgggcctgagggcttacctgcacggagctcaggacgcttacagggattgggagctgtcctaccctaacaccacatctttcggcctgtttctggtgaagccagacaacccctgggagtga

SEQ ID NO: 84 - protein, 177 aa, Homo sapiens
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 85 - DNA, 600 bp, Homo sapiens
  source 1..600: mol_type = genomic DNA
atgaccctgcacccaagccccatcacatgcgagttcctgttttctaccgccctgatcagcccaaagatgtgcctgagccacctggagaatatgcccctgtcccactctcggacacagggagcccagagaagctcctggaagctgtggctgttctgctctatcgtgatgctgctgttcctgtgcagcttttcctggctgatcttcatctttctgcagctggagacagccaaggagccttgcatggccaagtttggccctctgccatccaagtggcagatggcctctagcgagcccccttgcgtgaacaaggtgagcgactggaagctggagatcctgcagaacggcctgtacctgatctatggccaggtggcccccaacgccaattacaacgacgtggcccctttcgaggtgcggctgtataagaacaaggatatgatccagaccctgacaaataagtctaagatccagaacgtgggcggcacatacgagctgcacgtgggcgacaccatcgacctgatcttcaacagcgagcaccaggtgctgaagaacaatacatattggggcatcatcctgctggccaacccccagtttatctcctga

SEQ ID NO: 86 - skipped

SEQ ID NO: 87 - DNA, 1152 bp, Homo sapiens
  source 1..1152: mol_type = genomic DNA
atgctgctgttcctgctgtctgccctggtgctgctgacccagccactgggctacctggaggccgagatgaagacctattcccaccgcacaatgccttctgcctgcacactggtcatgtgcagcagcgtggagagcggcctgccaggaagggacggaagagatggaagggagggacccagaggcgagaagggcgaccctggcctgccaggagcagcaggacaggcaggaatgccaggccaggccggccccgtgggccctaagggcgacaatggatccgtgggagagccaggaccaaagggcgataccggcccttctggaccacctggaccaccaggcgtgcctggaccagcaggaagagagggacctctgggcaagcagggaaacatcggaccacagggcaagccaggccctaagggcgaggccggccccaagggcgaagtgggcgcccctggcatgcagggatccgccggagccaggggcctggccggacctaagggcgagcgcggcgtgcctggagagaggggcgtgccaggaaatacaggcgcagcaggatctgccggagcaatgggaccacagggcagccccggcgccagaggccctccaggcctgaagggcgacaagggaatccctggcgataagggagcaaagggagagagcggcctgccagacgtggcctccctgaggcagcaggtggaggccctgcagggacaggtgcagcacctgcaggccgccttcagccagtacaagaaggtggagctgtttccaaatggcgagacagccaaggagccctgcatggccaagttcggcccactgcccagcaagtggcagatggcctctagcgagcccccttgcgtgaacaaggtgagcgattggaagctggagatcctgcagaacggcctgtacctgatctatggccaggtggcccccaacgccaattacaacgacgtggccccttttgaggtgcggctgtataagaacaaggatatgatccagaccctgacaaataagtctaagatccagaacgtgggaggcacctacgagctgcacgtgggcgacacaatcgacctgatcttcaacagcgagcaccaggtgctgaagaacaatacatattggggcatcatcctgctggccaacccccagtttatctcctga

SEQ ID NO: 88 - protein, 173 aa, Mus musculus
MEEMPLRESSPQRAERCKKSWLLCIVALLLMLLCSLGTLIYTSLKPTAIESCMVKFELSSSKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKTNTYWGIILMPDLPFIS

SEQ ID NO: 89 - DNA, 522 bp, Mus musculus
  source 1..522: mol_type = genomic DNA
atggaggagatgcctctgagggagagctccccacagagggccgagagatgcaagaagagctggctgctgtgcatcgtggctctgctgctgatgctgctgtgctctctgggcaccctgatctacacaagcctgaagccaaccgccatcgagtcctgtatggtgaagttcgagctgtctagctccaagtggcacatgacatcccccaagcctcactgcgtgaacaccacatctgacggaaagctgaagatcctgcagagcggcacctacctgatctacggacaggtcatccccgtggacaagaagtacatcaaggataacgcccctttcgtggtgcagatctacaagaagaacgacgtgctgcagacactgatgaacgattttcagatcctgcccatcggcggagtgtacgagctgcacgctggcgacaacatctacctgaagttcaactccaaggatcacatccagaagaccaacacatactggggaatcatcctgatgccagatctgccctttatctcttga

SEQ ID NO: 90 - skipped

SEQ ID NO: 91 - DNA, 1164 bp, Mus musculus
  source 1..1164: mol_type = genomic DNA
atgctgcctttcctgtctatgctggtgctgctggtgcagccactgggcaacctgggagccgagatgaagagcctgtcccagagatccgtgcccaacacctgcacactggtcatgtgctctcctaccgagaacggcctgccaggaagggacggaagagatggaagggagggacctcggggagagaagggcgacccaggactgcctggaccaatgggactgagcggcctgcagggaccaacaggccccgtgggacctaagggagagaacggaagcgccggagagccaggacctaagggagagaggggactgtccggaccacctggactgcctggaatcccaggaccagctggcaaggagggaccatccggcaagcagggaaacatcggaccacagggaaagcctggaccaaagggagaggctggaccaaagggagaagtgggcgctcctggaatgcagggctccaccggagccaagggctctacaggaccaaaaggagagaggggagctcccggagtgcagggagcccctggcaacgctggagccgctggcccagccggacccgctggccctcagggagccccaggcagcaggggaccacccggcctgaagggcgacaggggcgtgccaggagataggggcatcaagggagagtctggcctgccagacagcgccgctctgagacagcagatggaggccctgaagggcaagctgcagcggctggaggtggctttctcccactaccagaaggccgctctgtttccagatggcagcctgaagcccaccgccatcgagtcctgcatggtgaagtttgagctgagctcctctaagtggcacatgacatctcccaagcctcactgcgtgaacaccacatctgacggcaagctgaagatcctgcagagcggcacctacctgatctacggccaggtcatccccgtggacaagaagtacatcaaggataacgcccctttcgtggtgcagatctacaagaagaacgacgtgctgcagacactgatgaacgattttcagatcctgccaatcggcggagtgtacgagctgcacgctggcgacaacatctacctgaagttcaactctaaggatcacatccagaagaccaacacatactggggcatcatcctgatgccagatctgccctttatcagctga

SEQ ID NO: 92 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 93 - DNA, 552 bp, Homo sapiens
  source 1..552: mol_type = genomic DNA
atggagagggtgcagcccctggaggagaacgtgggaaatgccgcccggcctagattcgagaggaacaagctgctgctggtggcctctgtgatccagggcctgggcctgctgctgtgcttcacctacatctgtctgcacttttctgccctgcaggtgagccacagatacccccgcatccagagcatcaaggtgcagttcaccgagtataagaaggagaagggctttatcctgacatcccagaaggaggacgagatcatgaaggtgcagaacaattctgtgatcatcaactgcgatggcttctacctgatctccctgaagggctatttttctcaggaagtgaatatcagcctgcactatcagaaggacgaggagccactgtttcagctgaagaaggtgcggagcgtgaattccctgatggtggccagcctgacctacaaggacaaggtgtatctgaacgtgaccacagataatacatccctggacgatttccacgtgaacggcggcgagctgatcctgatccaccagaatcccggcgagttttgcgtgctgtga

SEQ ID NO: 94 - skipped

SEQ ID NO: 95 - DNA, 1170 bp, Homo sapiens
  source 1..1170: mol_type = genomic DNA
atgctgctgttcctgctgagcgccctggtgctgctgacccagccactgggctacctggaggccgagatgaagacctattcccacagaacaatgccttctgcctgcacactggtcatgtgcagcagcgtggagtccggcctgccaggaagggacggcagagatggcagggagggccccaggggcgagaagggcgaccccggcctgcctggagcagcaggccaggccggcatgccaggccaggccggcccagtgggccccaagggcgacaacggcagcgtgggcgagcccggccctaagggcgataccggcccctccggcccccctggcccacccggcgtgccaggaccagcaggaagggagggaccactgggcaagcagggcaatatcggacctcagggcaagcctggaccaaagggagaggcaggaccaaagggagaagtgggcgcccctggcatgcagggatctgccggagcccggggcctggccggccccaagggcgagagaggcgtgcccggcgagaggggcgtgcctggcaacacaggcgccgccggctccgccggcgccatgggacctcagggctctccaggagccagaggccctccaggcctgaagggcgacaagggaatccctggcgataagggagcaaagggagagagcggcctgccagacgtggcctccctgcggcagcaggtggaggccctgcagggccaggtgcagcacctgcaggccgccttcagccagtacaagaaggtggagctgtttcctaatggcgtgtctcaccgctacccacggatccagagcatcaaggtgcagttcaccgagtataagaaggagaagggctttatcctgacatctcagaaggaggacgagatcatgaaggtgcagaacaatagcgtgatcatcaactgcgatggcttctacctgatcagcctgaagggctatttttcccaggaagtgaatatctctctgcactatcagaaggatgaggagcctctgtttcagctgaagaaggtgagatctgtgaacagcctgatggtggcctccctgacctacaaggacaaggtgtatctgaacgtgaccacagataatacatctctggacgatttccacgtgaacggcggcgagctgatcctgatccaccagaatcccggcgagttttgcgtgctgtga

SEQ ID NO: 96 - protein, 198 aa, Mus musculus
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 97 - DNA, 597 bp, Mus musculus
  source 1..597: mol_type = genomic DNA
atggagggcgagggagtgcagcccctggatgagaacctggagaacggctcccggcctcgcttcaagtggaagaagaccctgcggctggtggtgtctggaatcaagggcgccggaatgctgctgtgctttatctacgtgtgcctgcagctgagctcctctcccgccaaggatccccctatccagaggctgagaggagctgtgaccaggtgcgaggacggacagctgttcatcagctcctacaagaacgagtaccagacaatggaggtgcagaacaacagcgtggtcatcaagtgtgatggcctgtacatcatctacctgaagggatccttctttcaggaggtgaagatcgacctgcactttcgggaggatcacaacccaatctctatccccatgctgaacgacggcaggagaatcgtgttcacagtggtggccagcctggcttttaaggacaaggtgtacctgaccgtgaacgccccagatacactgtgcgagcacctgcagatcaacgacggagagctgatcgtggtgcagctgacccctggctactgtgctccagagggatcttaccacagcacagtgaaccaggtgcccctgtga

SEQ ID NO: 98 - skipped

SEQ ID NO: 99 - DNA, 1215 bp, Mus musculus
  source 1..1215: mol_type = genomic DNA
atgctgcccttcctgtccatgctggtgctgctggtgcagcctctgggcaacctgggagccgagatgaagtctctgagccagagatccgtgccaaacacctgcacactggtcatgtgctctcccaccgagaacggcctgcctggaagggacggaagagatggaagggagggaccccggggagagaagggcgatcctggactgccaggacctatgggactgagcggcctgcagggaccaacaggccccgtgggacctaagggagagaacggaagcgccggagagccaggaccaaagggagagaggggactgtccggcccacctggactgcctggaatccctggaccagctggcaaggagggaccttccggcaagcagggaaacatcggaccacagggaaagccaggacctaagggagaggctggaccaaagggagaagtgggcgctcccggaatgcagggctctaccggagccaagggcagcacaggacctaagggagagaggggagctccaggagtgcagggagcccccggcaacgctggagctgctggaccagctggaccagctggccctcagggagccccaggctctaggggaccaccaggcctgaagggcgacaggggcgtgccaggagataggggcatcaagggagagagcggcctgccagattccgccgctctgagacagcagatggaggccctgaagggcaagctgcagcggctggaggtggctttcagccactaccagaaggccgctctgtttcctgacggcagctcctctccagccaaggatcctccaatccagcggctgcgcggagctgtgaccaggtgcgaggatggccagctgttcatcagctcctacaagaacgagtaccagacaatggaggtgcagaacaactctgtggtcatcaagtgtgacggcctgtacatcatctacctgaagggcagcttctttcaggaggtgaagatcgacctgcactttagagaggatcacaacccaatctccatccccatgctgaacgacggcaggagaatcgtgttcaccgtggtggcctctctggcttttaaggacaaggtgtacctgaccgtgaacgcccccgatacactgtgcgagcacctgcagatcaacgacggcgagctgatcgtggtgcagctgacccctggatactgtgctccagagggctcctaccactctacagtgaaccaggtgcctctgtga

SEQ ID NO: 100 - protein, 302 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 101 - protein, 309 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTESWNLLLLLS

SEQ ID NO: 102 - protein, 185 aa, Homo sapiens
MRLGSPGLLFLLFSSLRAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 103 - protein, 217 aa, Homo sapiens
MSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 104 - protein, 275 aa, Homo sapiens
MVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 105 - DNA, 7078 bp, Homo sapiens
  source 1..7078: mol_type = genomic DNA
gcagttagagccgatctcccgcgccccgaggttgctcctctccgaggtctcccgcggcccaagttctccgcgccccgaggtctccgcgccccgaggtctccgcggcccgaggtctccgcccgcaccatgcggctgggcagtcctggactgctcttcctgctcttcagcagccttcgagctgatactcaggagaaggaagtcagagcgatggtaggcagcgacgtggagctcagctgcgcttgccctgaaggaagccgttttgatttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccaggacgagcagaagtttcactgcctggtgttgagccaatccctgggattccaggaggttttgagcgttgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactggccacgtttgaccggagctcaccgcccagagcgtggacagggcttccgtgagacgccaccgtgagaggccaggtggcagcttgagcatggactcccagactgcaggggagcacttggggcagcccccagaaggaccactgctggatcccagggagaacctgctggcgttggctgtgatcctggaatgaggccctttcaaaagcgtcatccacaccaaaggcaaatgtccccaagtgagtgggctccccgctgtcactgccagtcacccacaggaagggactggtgatgggctgtctctacccggagcgtgcgggattcagcaccaggctcttcccagtaccccagacccactgtgggtcttcccgtgggatgcgggatcctgagaccgaagggtgtttggtttaaaaagaagactgggcgtccgctcttccaggacggcctctgtgctgctggggtcacgcgaggctgtttgcaggggacacggtcacaggagctcttctgccctgaacgctcccaacctgcctcccgcccggaagccacaggacccactcatgtgtgtgcccacaagtgtagttagccgtccacaccgaggagcccccggaagtccccactgggcttcagtgtcctctgccacattccctgggaggaacaatgtccctcggctgttccggtgaaaagttgagccacctttggaagacgcacgggtggagtttgccagaagaaaggctgtgccagggccgtgtttggctacaggggctgccggggctcttggctctgcagcgagaaagacacagcccagcagggctggagacgcccatgtccagcaggcgcaggcctggcaacacggtccccagagtcctgagcagcagttaggtgcatggagagggtatcacctggtggccacagtcccccttctcacctcagcaatgatccccaaagtgagaggtggctcccccggcccccaccaccctcagcagccccaccccactcaaccctgagggtccccagggtcctgatgaagacctccgaccccagcgccaggctcctcggagcccaacagtcccaagggggcaggagacggggtggtccagtgctgaggggtacagccctgggccctgaccagccccggcacctgccatgctggttcccggaatgaatcagctgctgactgtctccagaagggctggaaaggatgctgccaggtgacccgaggtgcactcgccccagggagatggagtagacagcctggcctggccctcgggacacattgtctgccccggggctatgggcaaatgcccctccttcttacttcccagaatcccctgacattcccagggtcagccaggacctgttacagccctggtcacttggaactgacagctgtgtgaggcctgcacttctcagacccagacttagaacaaaaggaggagtgaggactcaaggctacaatgaggttccagtacttgttacaagaaattggttttctgcaaaaaaagtccctacctgagcctttaggtgaatgtgggatccactcccgcttttaacatgaaagcattagaagatgtgtggtgtttataaaagaacagttgtcatcaccgggcattgattggcagggacaaggagctgcttgggtgtggaaagttggggcgttggaaagtgggctgtggtgcccatttgcagtgactgtgaagtgactccaggacggacctgcgggggcacccagaggtcctaagccccaggactgagggtcgtgcatcaccactcgggtgtcccgggaggtgccctgggcccggggacctcacaggcaggacggcgacactaatgcagggagagggagtctggccccagcttttcctatcagaggcgattttccttcaccaggggatgggcaggaaagaggcaggggccccagaagcttctgtccctcatgcctgagggcacgggggacacttggaggctgctgtcaccactgtgcgtccaaggccatgctctctgcgggtcagtgcctgagtctcgcctccctgctggtccctgaagccccctcagaagccctgcctgtcacgtcggcatttgtgagacctaccctgtaacgcctgcccctctcagcccaacatcagcttcctctttctcccttgctgtagacaggctggattccagtgttgggacagccatctccagaaacctgacttaagagagtaagatgcaaatcgtgcctgtatccagtggctttggtgggtgcagggagtcttgggcacagccagctcagctgtctgtggtatgagcaggaacaggtgccactcctgctcaggggaccctgccctacaccaggctgttccgtccccctggaggacatggggccaggtctggaggcattttgggttgtcacagctgggggctgttcctcggcttcagcgggtggaagcctcagatgctgttcaacatcttctggacacgggaggccccgacagagagaagcgtccacccgcaagtccacagtctgaggtctcccctcagagaccctgccctgcacacccacctccagccaaaggtcctgcctgccccagggctcaggggaaccttgccggtctgtggaacaggagaggggactctcgccagctgcaccaccctgcacgtagtaggtgtgcggtaaacatccaccagggaggctccagtcaaggctggcagatggggcggtccatccctagggcaggtgacagaagggaaaaggctgcctgctggcccccgagccaggtagcacatgcttgtgcctcagtttcccctcctgtaaagtgaggcgctggatccaggttctgtctactgggctctgcagcttggacgctcctaagaccaagcgacccaccctggggagggcagctatggctttggaatagctgtccaggcccgggtgcctccaagacggccaccacaccctgcctgtgctgcaggggtgcaggggtaaggggcaagactccagaggcctcctctctgcatctccttgtcttcagtggccggaggtgaggcctgagctcaggggaggggcttctgccacgaaccctatggcggggcacagcacacttttcccagggaggacccctgggccccctgcattatccccagcggagtgtggggtcaccttccaagagcgacattgagaagctccagctctaggagtgtgcagactcttaaccaggcaggcccaggccctggggcacacaaaggcggggcctgctctccccagctgcccctgccaatgggggctggactgtcctaccctcctcccttctacctccccactgtcttccctctccactgtcaccactgcctccctcttccactgtcctccatgcactgccctccctccaccttcccccacccccaccactccccatgctgtccccaggctccccccgctctcccccctccccactgtccccctccccatgctgtacccagctcaccccgctctcccctctccccactgtcccccctcccactccccatgctgtccccagctcaccctacatggacttggcgatgtccttccatggctcaccggtctgaatttccatgatgagccgggcctgcagctttgctcccctatccctgcccaggctgcagctgtccatgcagggagcgagctccagcacctgcggagtccttccgtgggggcctctccgtgccacagcagccagggacctcaggtgcctgtgcatgacaccaccgcccatcctcatcctgagccagcctctcaggatcaggacttggtttggcggcgttaaccttagagcctgcaaggggcttcctcctggtgggtctggccgtagcctggggaggccacagctccaggccactccagacctcccttcctctgggccttccatgtggtggcaaccaccgcagctgtaagggagggaaaatggagcgtttgttctcgggctgggctggggtctgggggaagccatgggcgtgaagactggagtattatttgatggagaagcggccactcctggagaccggcggcaaacacagaagcacagcgtggaaggtgctggtgtcagcccacacgggtgatggggtcagactcaggagtcacactcaggagtcaccaggctcaaagggcccaggcaccgcaagtcctgctcagccccagacacaatgcattcctgttgccctcgccctcagccaggccccacgcaggccagggagcactggcaaagcttggcaaccctctgggggccagccttcatccaggccgaaggtggtcagtggcccaccatggcccaggtagaaaactcacggattaagatttcatgcccgactccaaaggcaagagactttattattttattttttttgagccagagtatcgctctgtcacctaggctggagtgcaatctctgctcattgcaacatctgcctcccgaactcaagcaattctgcctcagcctcccaagtagctgggattacaggtgtgcgccaccatgcccaggtaattgtatttttagtagagacagggtttcaccatgttggtcaggctggtttcaaactcctgacctcaaatgatctgcccacctcgacctcccaaagtgctgggattacaggtgcgagccaccgcacctggctaccagacacttcagagttacaggttagtttttctttttcttttatttttttttttttggcggaggtgcagggggagttaaacaaacaaacaaaataaacaggccgggtgcggtggctcatgcctgtaatcccagcactttaggaggcctaggtgggtggatcacgagatcaggggttcaagaccagcctggccgagatggtaaaaccccgtctccactaaaaatacaaaaattggccaggcacggtggctcacacctgtaatcccagtactttgggaggctgaggtgggcagatcacctgaggtcaggagttcaagaccaacctgaccaacatggagaaaccccatctctactaaaaatacaaaattagccaggtgtggtggtgcatgcctgtaattccagctactcgggaggctgaggcaggagaattgcttgaacccaggaggcagaggttgcagtgggccaagatggcgccattgcactccagcctgggaacaagagcgaaactctgactaaaaaagaaagaaagaaagaaaaaaattagttgggcacggtggcaggcgcctgtaatcccaggtactcaggaggctgaggcaggagaattgcttgaacccgggaggcagaggtcgcagtgagccgagattgcaccactgccctccagcctgggtgacagagcaagactccgtctcaaaaaaaaaaaaaaaaaaaattggatacattgtaatacctcaaatacttgtaagtgaagcaccccagttcccatagagctgccgcactcagaggcttctgtaacctgcctgctcccagcattctatttagggtctggtatgtccagaatttgcagacacagcaattcctgcagcagcagtgcaccatgtggaaggggccccatgaccagcccactgtgagctcacacgtgatgactgaggcttcttcacacagcagggctctgggtgtgatacccagggcacacgcgtttgcacaggcacaggccacacaagttctcacatgctcagccccataagccgtgctggacaggcatggccatttacacccaggatcctgctgagaacagcaaccaactcaccaccctcgcatcatgatccttgccacacaggggctctggtggctttggtggcctgggctgtggctctgctgccagccaccttgagtgaagatccgggttctctgggtgctactcagctgctatgtggggagctggcccctggggtgatgagggcccttcccaacccgccctcagcccttggacagccaggatcacccggggctgtctgcatacagacttctcaggggagttctcagcttggacccttatctccccagaatcctggaacctgctccttctgctctcgtgactgactgtgttctctatgcaacttccaataaaacctcttcatttgaaaggaaaaaa

SEQ ID NO: 106 - DNA, 1112 bp, Homo sapiens
  source 1..1112: mol_type = genomic DNA
gcagttagagccgatctcccgcgccccgaggttgctcctctccgaggtctcccgcggcccaagttctccgcgccccgaggtctccgcgccccgaggtctccgcggcccgaggtctccgcccgcaccatgcggctgggcagtcctggactgctcttcctgctcttcagcagccttcgagctgatactcaggagaaggaagtcagagcgatggtaggcagcgacgtggagctcagctgcgcttgccctgaaggaagccgttttgatttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccaggacgagcagaagtttcactgcctggtgttgagccaatccctgggattccaggaggttttgagcgttgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactgaatcctggaacctgctccttctgctctcgtgactgactgtgttctctatgcaacttccaataaaacctcttcatttgaaaggaaaaaa

SEQ ID NO: 107 - DNA, 6727 bp, Homo sapiens
  source 1..6727: mol_type = genomic DNA
gcagttagagccgatctcccgcgccccgaggttgctcctctccgaggtctcccgcggcccaagttctccgcgccccgaggtctccgcgccccgaggtctccgcggcccgaggtctccgcccgcaccatgcggctgggcagtcctggactgctcttcctgctcttcagcagccttcgagctgcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactggccacgtttgaccggagctcaccgcccagagcgtggacagggcttccgtgagacgccaccgtgagaggccaggtggcagcttgagcatggactcccagactgcaggggagcacttggggcagcccccagaaggaccactgctggatcccagggagaacctgctggcgttggctgtgatcctggaatgaggccctttcaaaagcgtcatccacaccaaaggcaaatgtccccaagtgagtgggctccccgctgtcactgccagtcacccacaggaagggactggtgatgggctgtctctacccggagcgtgcgggattcagcaccaggctcttcccagtaccccagacccactgtgggtcttcccgtgggatgcgggatcctgagaccgaagggtgtttggtttaaaaagaagactgggcgtccgctcttccaggacggcctctgtgctgctggggtcacgcgaggctgtttgcaggggacacggtcacaggagctcttctgccctgaacgctcccaacctgcctcccgcccggaagccacaggacccactcatgtgtgtgcccacaagtgtagttagccgtccacaccgaggagcccccggaagtccccactgggcttcagtgtcctctgccacattccctgggaggaacaatgtccctcggctgttccggtgaaaagttgagccacctttggaagacgcacgggtggagtttgccagaagaaaggctgtgccagggccgtgtttggctacaggggctgccggggctcttggctctgcagcgagaaagacacagcccagcagggctggagacgcccatgtccagcaggcgcaggcctggcaacacggtccccagagtcctgagcagcagttaggtgcatggagagggtatcacctggtggccacagtcccccttctcacctcagcaatgatccccaaagtgagaggtggctcccccggcccccaccaccctcagcagccccaccccactcaaccctgagggtccccagggtcctgatgaagacctccgaccccagcgccaggctcctcggagcccaacagtcccaagggggcaggagacggggtggtccagtgctgaggggtacagccctgggccctgaccagccccggcacctgccatgctggttcccggaatgaatcagctgctgactgtctccagaagggctggaaaggatgctgccaggtgacccgaggtgcactcgccccagggagatggagtagacagcctggcctggccctcgggacacattgtctgccccggggctatgggcaaatgcccctccttcttacttcccagaatcccctgacattcccagggtcagccaggacctgttacagccctggtcacttggaactgacagctgtgtgaggcctgcacttctcagacccagacttagaacaaaaggaggagtgaggactcaaggctacaatgaggttccagtacttgttacaagaaattggttttctgcaaaaaaagtccctacctgagcctttaggtgaatgtgggatccactcccgcttttaacatgaaagcattagaagatgtgtggtgtttataaaagaacagttgtcatcaccgggcattgattggcagggacaaggagctgcttgggtgtggaaagttggggcgttggaaagtgggctgtggtgcccatttgcagtgactgtgaagtgactccaggacggacctgcgggggcacccagaggtcctaagccccaggactgagggtcgtgcatcaccactcgggtgtcccgggaggtgccctgggcccggggacctcacaggcaggacggcgacactaatgcagggagagggagtctggccccagcttttcctatcagaggcgattttccttcaccaggggatgggcaggaaagaggcaggggccccagaagcttctgtccctcatgcctgagggcacgggggacacttggaggctgctgtcaccactgtgcgtccaaggccatgctctctgcgggtcagtgcctgagtctcgcctccctgctggtccctgaagccccctcagaagccctgcctgtcacgtcggcatttgtgagacctaccctgtaacgcctgcccctctcagcccaacatcagcttcctctttctcccttgctgtagacaggctggattccagtgttgggacagccatctccagaaacctgacttaagagagtaagatgcaaatcgtgcctgtatccagtggctttggtgggtgcagggagtcttgggcacagccagctcagctgtctgtggtatgagcaggaacaggtgccactcctgctcaggggaccctgccctacaccaggctgttccgtccccctggaggacatggggccaggtctggaggcattttgggttgtcacagctgggggctgttcctcggcttcagcgggtggaagcctcagatgctgttcaacatcttctggacacgggaggccccgacagagagaagcgtccacccgcaagtccacagtctgaggtctcccctcagagaccctgccctgcacacccacctccagccaaaggtcctgcctgccccagggctcaggggaaccttgccggtctgtggaacaggagaggggactctcgccagctgcaccaccctgcacgtagtaggtgtgcggtaaacatccaccagggaggctccagtcaaggctggcagatggggcggtccatccctagggcaggtgacagaagggaaaaggctgcctgctggcccccgagccaggtagcacatgcttgtgcctcagtttcccctcctgtaaagtgaggcgctggatccaggttctgtctactgggctctgcagcttggacgctcctaagaccaagcgacccaccctggggagggcagctatggctttggaatagctgtccaggcccgggtgcctccaagacggccaccacaccctgcctgtgctgcaggggtgcaggggtaaggggcaagactccagaggcctcctctctgcatctccttgtcttcagtggccggaggtgaggcctgagctcaggggaggggcttctgccacgaaccctatggcggggcacagcacacttttcccagggaggacccctgggccccctgcattatccccagcggagtgtggggtcaccttccaagagcgacattgagaagctccagctctaggagtgtgcagactcttaaccaggcaggcccaggccctggggcacacaaaggcggggcctgctctccccagctgcccctgccaatgggggctggactgtcctaccctcctcccttctacctccccactgtcttccctctccactgtcaccactgcctccctcttccactgtcctccatgcactgccctccctccaccttcccccacccccaccactccccatgctgtccccaggctccccccgctctcccccctccccactgtccccctccccatgctgtacccagctcaccccgctctcccctctccccactgtcccccctcccactccccatgctgtccccagctcaccctacatggacttggcgatgtccttccatggctcaccggtctgaatttccatgatgagccgggcctgcagctttgctcccctatccctgcccaggctgcagctgtccatgcagggagcgagctccagcacctgcggagtccttccgtgggggcctctccgtgccacagcagccagggacctcaggtgcctgtgcatgacaccaccgcccatcctcatcctgagccagcctctcaggatcaggacttggtttggcggcgttaaccttagagcctgcaaggggcttcctcctggtgggtctggccgtagcctggggaggccacagctccaggccactccagacctcccttcctctgggccttccatgtggtggcaaccaccgcagctgtaagggagggaaaatggagcgtttgttctcgggctgggctggggtctgggggaagccatgggcgtgaagactggagtattatttgatggagaagcggccactcctggagaccggcggcaaacacagaagcacagcgtggaaggtgctggtgtcagcccacacgggtgatggggtcagactcaggagtcacactcaggagtcaccaggctcaaagggcccaggcaccgcaagtcctgctcagccccagacacaatgcattcctgttgccctcgccctcagccaggccccacgcaggccagggagcactggcaaagcttggcaaccctctgggggccagccttcatccaggccgaaggtggtcagtggcccaccatggcccaggtagaaaactcacggattaagatttcatgcccgactccaaaggcaagagactttattattttattttttttgagccagagtatcgctctgtcacctaggctggagtgcaatctctgctcattgcaacatctgcctcccgaactcaagcaattctgcctcagcctcccaagtagctgggattacaggtgtgcgccaccatgcccaggtaattgtatttttagtagagacagggtttcaccatgttggtcaggctggtttcaaactcctgacctcaaatgatctgcccacctcgacctcccaaagtgctgggattacaggtgcgagccaccgcacctggctaccagacacttcagagttacaggttagtttttctttttcttttatttttttttttttggcggaggtgcagggggagttaaacaaacaaacaaaataaacaggccgggtgcggtggctcatgcctgtaatcccagcactttaggaggcctaggtgggtggatcacgagatcaggggttcaagaccagcctggccgagatggtaaaaccccgtctccactaaaaatacaaaaattggccaggcacggtggctcacacctgtaatcccagtactttgggaggctgaggtgggcagatcacctgaggtcaggagttcaagaccaacctgaccaacatggagaaaccccatctctactaaaaatacaaaattagccaggtgtggtggtgcatgcctgtaattccagctactcgggaggctgaggcaggagaattgcttgaacccaggaggcagaggttgcagtgggccaagatggcgccattgcactccagcctgggaacaagagcgaaactctgactaaaaaagaaagaaagaaagaaaaaaattagttgggcacggtggcaggcgcctgtaatcccaggtactcaggaggctgaggcaggagaattgcttgaacccgggaggcagaggtcgcagtgagccgagattgcaccactgccctccagcctgggtgacagagcaagactccgtctcaaaaaaaaaaaaaaaaaaaattggatacattgtaatacctcaaatacttgtaagtgaagcaccccagttcccatagagctgccgcactcagaggcttctgtaacctgcctgctcccagcattctatttagggtctggtatgtccagaatttgcagacacagcaattcctgcagcagcagtgcaccatgtggaaggggccccatgaccagcccactgtgagctcacacgtgatgactgaggcttcttcacacagcagggctctgggtgtgatacccagggcacacgcgtttgcacaggcacaggccacacaagttctcacatgctcagccccataagccgtgctggacaggcatggccatttacacccaggatcctgctgagaacagcaaccaactcaccaccctcgcatcatgatccttgccacacaggggctctggtggctttggtggcctgggctgtggctctgctgccagccaccttgagtgaagatccgggttctctgggtgctactcagctgctatgtggggagctggcccctggggtgatgagggcccttcccaacccgccctcagcccttggacagccaggatcacccggggctgtctgcatacagacttctcaggggagttctcagcttggacccttatctccccagaatcctggaacctgctccttctgctctcgtgactgactgtgttctctatgcaacttccaataaaacctcttcatttgaaaggaaaaaa

SEQ ID NO: 108 - DNA, 6926 bp, Homo sapiens
  source 1..6926: mol_type = genomic DNA
gcagttagagccgatctcccgcgccccgaggttgctcctctccgaggtctcccgcggcccaagttctccgcgccccgaggtctccgcgccccgaggtctccgcggcccgaggtctccgcccgcaccatgcggctgggcagtcctggactgctcttcctgctcttcagcagccttcgagctgaacagctccttggaaaacgtggacagccgctaccggaaccgagccctgatgtcaccggccggcatgctgcggggcgacttctccctgcgcttgttcaacgtcaccccccaggacgagcagaagtttcactgcctggtgttgagccaatccctgggattccaggaggttttgagcgttgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactggccacgtttgaccggagctcaccgcccagagcgtggacagggcttccgtgagacgccaccgtgagaggccaggtggcagcttgagcatggactcccagactgcaggggagcacttggggcagcccccagaaggaccactgctggatcccagggagaacctgctggcgttggctgtgatcctggaatgaggccctttcaaaagcgtcatccacaccaaaggcaaatgtccccaagtgagtgggctccccgctgtcactgccagtcacccacaggaagggactggtgatgggctgtctctacccggagcgtgcgggattcagcaccaggctcttcccagtaccccagacccactgtgggtcttcccgtgggatgcgggatcctgagaccgaagggtgtttggtttaaaaagaagactgggcgtccgctcttccaggacggcctctgtgctgctggggtcacgcgaggctgtttgcaggggacacggtcacaggagctcttctgccctgaacgctcccaacctgcctcccgcccggaagccacaggacccactcatgtgtgtgcccacaagtgtagttagccgtccacaccgaggagcccccggaagtccccactgggcttcagtgtcctctgccacattccctgggaggaacaatgtccctcggctgttccggtgaaaagttgagccacctttggaagacgcacgggtggagtttgccagaagaaaggctgtgccagggccgtgtttggctacaggggctgccggggctcttggctctgcagcgagaaagacacagcccagcagggctggagacgcccatgtccagcaggcgcaggcctggcaacacggtccccagagtcctgagcagcagttaggtgcatggagagggtatcacctggtggccacagtcccccttctcacctcagcaatgatccccaaagtgagaggtggctcccccggcccccaccaccctcagcagccccaccccactcaaccctgagggtccccagggtcctgatgaagacctccgaccccagcgccaggctcctcggagcccaacagtcccaagggggcaggagacggggtggtccagtgctgaggggtacagccctgggccctgaccagccccggcacctgccatgctggttcccggaatgaatcagctgctgactgtctccagaagggctggaaaggatgctgccaggtgacccgaggtgcactcgccccagggagatggagtagacagcctggcctggccctcgggacacattgtctgccccggggctatgggcaaatgcccctccttcttacttcccagaatcccctgacattcccagggtcagccaggacctgttacagccctggtcacttggaactgacagctgtgtgaggcctgcacttctcagacccagacttagaacaaaaggaggagtgaggactcaaggctacaatgaggttccagtacttgttacaagaaattggttttctgcaaaaaaagtccctacctgagcctttaggtgaatgtgggatccactcccgcttttaacatgaaagcattagaagatgtgtggtgtttataaaagaacagttgtcatcaccgggcattgattggcagggacaaggagctgcttgggtgtggaaagttggggcgttggaaagtgggctgtggtgcccatttgcagtgactgtgaagtgactccaggacggacctgcgggggcacccagaggtcctaagccccaggactgagggtcgtgcatcaccactcgggtgtcccgggaggtgccctgggcccggggacctcacaggcaggacggcgacactaatgcagggagagggagtctggccccagcttttcctatcagaggcgattttccttcaccaggggatgggcaggaaagaggcaggggccccagaagcttctgtccctcatgcctgagggcacgggggacacttggaggctgctgtcaccactgtgcgtccaaggccatgctctctgcgggtcagtgcctgagtctcgcctccctgctggtccctgaagccccctcagaagccctgcctgtcacgtcggcatttgtgagacctaccctgtaacgcctgcccctctcagcccaacatcagcttcctctttctcccttgctgtagacaggctggattccagtgttgggacagccatctccagaaacctgacttaagagagtaagatgcaaatcgtgcctgtatccagtggctttggtgggtgcagggagtcttgggcacagccagctcagctgtctgtggtatgagcaggaacaggtgccactcctgctcaggggaccctgccctacaccaggctgttccgtccccctggaggacatggggccaggtctggaggcattttgggttgtcacagctgggggctgttcctcggcttcagcgggtggaagcctcagatgctgttcaacatcttctggacacgggaggccccgacagagagaagcgtccacccgcaagtccacagtctgaggtctcccctcagagaccctgccctgcacacccacctccagccaaaggtcctgcctgccccagggctcaggggaaccttgccggtctgtggaacaggagaggggactctcgccagctgcaccaccctgcacgtagtaggtgtgcggtaaacatccaccagggaggctccagtcaaggctggcagatggggcggtccatccctagggcaggtgacagaagggaaaaggctgcctgctggcccccgagccaggtagcacatgcttgtgcctcagtttcccctcctgtaaagtgaggcgctggatccaggttctgtctactgggctctgcagcttggacgctcctaagaccaagcgacccaccctggggagggcagctatggctttggaatagctgtccaggcccgggtgcctccaagacggccaccacaccctgcctgtgctgcaggggtgcaggggtaaggggcaagactccagaggcctcctctctgcatctccttgtcttcagtggccggaggtgaggcctgagctcaggggaggggcttctgccacgaaccctatggcggggcacagcacacttttcccagggaggacccctgggccccctgcattatccccagcggagtgtggggtcaccttccaagagcgacattgagaagctccagctctaggagtgtgcagactcttaaccaggcaggcccaggccctggggcacacaaaggcggggcctgctctccccagctgcccctgccaatgggggctggactgtcctaccctcctcccttctacctccccactgtcttccctctccactgtcaccactgcctccctcttccactgtcctccatgcactgccctccctccaccttcccccacccccaccactccccatgctgtccccaggctccccccgctctcccccctccccactgtccccctccccatgctgtacccagctcaccccgctctcccctctccccactgtcccccctcccactccccatgctgtccccagctcaccctacatggacttggcgatgtccttccatggctcaccggtctgaatttccatgatgagccgggcctgcagctttgctcccctatccctgcccaggctgcagctgtccatgcagggagcgagctccagcacctgcggagtccttccgtgggggcctctccgtgccacagcagccagggacctcaggtgcctgtgcatgacaccaccgcccatcctcatcctgagccagcctctcaggatcaggacttggtttggcggcgttaaccttagagcctgcaaggggcttcctcctggtgggtctggccgtagcctggggaggccacagctccaggccactccagacctcccttcctctgggccttccatgtggtggcaaccaccgcagctgtaagggagggaaaatggagcgtttgttctcgggctgggctggggtctgggggaagccatgggcgtgaagactggagtattatttgatggagaagcggccactcctggagaccggcggcaaacacagaagcacagcgtggaaggtgctggtgtcagcccacacgggtgatggggtcagactcaggagtcacactcaggagtcaccaggctcaaagggcccaggcaccgcaagtcctgctcagccccagacacaatgcattcctgttgccctcgccctcagccaggccccacgcaggccagggagcactggcaaagcttggcaaccctctgggggccagccttcatccaggccgaaggtggtcagtggcccaccatggcccaggtagaaaactcacggattaagatttcatgcccgactccaaaggcaagagactttattattttattttttttgagccagagtatcgctctgtcacctaggctggagtgcaatctctgctcattgcaacatctgcctcccgaactcaagcaattctgcctcagcctcccaagtagctgggattacaggtgtgcgccaccatgcccaggtaattgtatttttagtagagacagggtttcaccatgttggtcaggctggtttcaaactcctgacctcaaatgatctgcccacctcgacctcccaaagtgctgggattacaggtgcgagccaccgcacctggctaccagacacttcagagttacaggttagtttttctttttcttttatttttttttttttggcggaggtgcagggggagttaaacaaacaaacaaaataaacaggccgggtgcggtggctcatgcctgtaatcccagcactttaggaggcctaggtgggtggatcacgagatcaggggttcaagaccagcctggccgagatggtaaaaccccgtctccactaaaaatacaaaaattggccaggcacggtggctcacacctgtaatcccagtactttgggaggctgaggtgggcagatcacctgaggtcaggagttcaagaccaacctgaccaacatggagaaaccccatctctactaaaaatacaaaattagccaggtgtggtggtgcatgcctgtaattccagctactcgggaggctgaggcaggagaattgcttgaacccaggaggcagaggttgcagtgggccaagatggcgccattgcactccagcctgggaacaagagcgaaactctgactaaaaaagaaagaaagaaagaaaaaaattagttgggcacggtggcaggcgcctgtaatcccaggtactcaggaggctgaggcaggagaattgcttgaacccgggaggcagaggtcgcagtgagccgagattgcaccactgccctccagcctgggtgacagagcaagactccgtctcaaaaaaaaaaaaaaaaaaaattggatacattgtaatacctcaaatacttgtaagtgaagcaccccagttcccatagagctgccgcactcagaggcttctgtaacctgcctgctcccagcattctatttagggtctggtatgtccagaatttgcagacacagcaattcctgcagcagcagtgcaccatgtggaaggggccccatgaccagcccactgtgagctcacacgtgatgactgaggcttcttcacacagcagggctctgggtgtgatacccagggcacacgcgtttgcacaggcacaggccacacaagttctcacatgctcagccccataagccgtgctggacaggcatggccatttacacccaggatcctgctgagaacagcaaccaactcaccaccctcgcatcatgatccttgccacacaggggctctggtggctttggtggcctgggctgtggctctgctgccagccaccttgagtgaagatccgggttctctgggtgctactcagctgctatgtggggagctggcccctggggtgatgagggcccttcccaacccgccctcagcccttggacagccaggatcacccggggctgtctgcatacagacttctcaggggagttctcagcttggacccttatctccccagaatcctggaacctgctccttctgctctcgtgactgactgtgttctctatgcaacttccaataaaacctcttcatttgaaaggaaaaaa

SEQ ID NO: 109 - DNA, 7414 bp, Homo sapiens
  source 1..7414: mol_type = genomic DNA
gcagttagagccgatctcccgcgccccgaggttgctcctctccgaggtctcccgcggcccaagttctccgcgccccgaggtctccgcgccccgaggtctccgcggcccgaggtctccgcccgcaccatgcggctgggcaggaaggctagaccagcctcactggcgtccctcgctccccttcatcctcacgccagcatcactgtgtttctggggatttgttaccgcgagagctagagagtggtgagaggctgccagcccctcggacttgctgtgctgggcaggagggtggtggtcctggccccggaccccgaattcccatggagaacagaggcaggcaggctcctggctctgccttcacatttctgtgatgggcttaactacctcctgctcatgggaaccagcctgttcctgtttggaagatacaggtaactccctggtggccccgcctcacctgcctttctggttgttttttccagtcctggactgctcttcctgctcttcagcagccttcgagctgatactcaggagaaggaagtcagagcgatggtaggcagcgacgtggagctcagctgcgcttgccctgaaggaagccgttttgatttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccaggacgagcagaagtttcactgcctggtgttgagccaatccctgggattccaggaggttttgagcgttgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactggccacgtttgaccggagctcaccgcccagagcgtggacagggcttccgtgagacgccaccgtgagaggccaggtggcagcttgagcatggactcccagactgcaggggagcacttggggcagcccccagaaggaccactgctggatcccagggagaacctgctggcgttggctgtgatcctggaatgaggccctttcaaaagcgtcatccacaccaaaggcaaatgtccccaagtgagtgggctccccgctgtcactgccagtcacccacaggaagggactggtgatgggctgtctctacccggagcgtgcgggattcagcaccaggctcttcccagtaccccagacccactgtgggtcttcccgtgggatgcgggatcctgagaccgaagggtgtttggtttaaaaagaagactgggcgtccgctcttccaggacggcctctgtgctgctggggtcacgcgaggctgtttgcaggggacacggtcacaggagctcttctgccctgaacgctcccaacctgcctcccgcccggaagccacaggacccactcatgtgtgtgcccacaagtgtagttagccgtccacaccgaggagcccccggaagtccccactgggcttcagtgtcctctgccacattccctgggaggaacaatgtccctcggctgttccggtgaaaagttgagccacctttggaagacgcacgggtggagtttgccagaagaaaggctgtgccagggccgtgtttggctacaggggctgccggggctcttggctctgcagcgagaaagacacagcccagcagggctggagacgcccatgtccagcaggcgcaggcctggcaacacggtccccagagtcctgagcagcagttaggtgcatggagagggtatcacctggtggccacagtcccccttctcacctcagcaatgatccccaaagtgagaggtggctcccccggcccccaccaccctcagcagccccaccccactcaaccctgagggtccccagggtcctgatgaagacctccgaccccagcgccaggctcctcggagcccaacagtcccaagggggcaggagacggggtggtccagtgctgaggggtacagccctgggccctgaccagccccggcacctgccatgctggttcccggaatgaatcagctgctgactgtctccagaagggctggaaaggatgctgccaggtgacccgaggtgcactcgccccagggagatggagtagacagcctggcctggccctcgggacacattgtctgccccggggctatgggcaaatgcccctccttcttacttcccagaatcccctgacattcccagggtcagccaggacctgttacagccctggtcacttggaactgacagctgtgtgaggcctgcacttctcagacccagacttagaacaaaaggaggagtgaggactcaaggctacaatgaggttccagtacttgttacaagaaattggttttctgcaaaaaaagtccctacctgagcctttaggtgaatgtgggatccactcccgcttttaacatgaaagcattagaagatgtgtggtgtttataaaagaacagttgtcatcaccgggcattgattggcagggacaaggagctgcttgggtgtggaaagttggggcgttggaaagtgggctgtggtgcccatttgcagtgactgtgaagtgactccaggacggacctgcgggggcacccagaggtcctaagccccaggactgagggtcgtgcatcaccactcgggtgtcccgggaggtgccctgggcccggggacctcacaggcaggacggcgacactaatgcagggagagggagtctggccccagcttttcctatcagaggcgattttccttcaccaggggatgggcaggaaagaggcaggggccccagaagcttctgtccctcatgcctgagggcacgggggacacttggaggctgctgtcaccactgtgcgtccaaggccatgctctctgcgggtcagtgcctgagtctcgcctccctgctggtccctgaagccccctcagaagccctgcctgtcacgtcggcatttgtgagacctaccctgtaacgcctgcccctctcagcccaacatcagcttcctctttctcccttgctgtagacaggctggattccagtgttgggacagccatctccagaaacctgacttaagagagtaagatgcaaatcgtgcctgtatccagtggctttggtgggtgcagggagtcttgggcacagccagctcagctgtctgtggtatgagcaggaacaggtgccactcctgctcaggggaccctgccctacaccaggctgttccgtccccctggaggacatggggccaggtctggaggcattttgggttgtcacagctgggggctgttcctcggcttcagcgggtggaagcctcagatgctgttcaacatcttctggacacgggaggccccgacagagagaagcgtccacccgcaagtccacagtctgaggtctcccctcagagaccctgccctgcacacccacctccagccaaaggtcctgcctgccccagggctcaggggaaccttgccggtctgtggaacaggagaggggactctcgccagctgcaccaccctgcacgtagtaggtgtgcggtaaacatccaccagggaggctccagtcaaggctggcagatggggcggtccatccctagggcaggtgacagaagggaaaaggctgcctgctggcccccgagccaggtagcacatgcttgtgcctcagtttcccctcctgtaaagtgaggcgctggatccaggttctgtctactgggctctgcagcttggacgctcctaagaccaagcgacccaccctggggagggcagctatggctttggaatagctgtccaggcccgggtgcctccaagacggccaccacaccctgcctgtgctgcaggggtgcaggggtaaggggcaagactccagaggcctcctctctgcatctccttgtcttcagtggccggaggtgaggcctgagctcaggggaggggcttctgccacgaaccctatggcggggcacagcacacttttcccagggaggacccctgggccccctgcattatccccagcggagtgtggggtcaccttccaagagcgacattgagaagctccagctctaggagtgtgcagactcttaaccaggcaggcccaggccctggggcacacaaaggcggggcctgctctccccagctgcccctgccaatgggggctggactgtcctaccctcctcccttctacctccccactgtcttccctctccactgtcaccactgcctccctcttccactgtcctccatgcactgccctccctccaccttcccccacccccaccactccccatgctgtccccaggctccccccgctctcccccctccccactgtccccctccccatgctgtacccagctcaccccgctctcccctctccccactgtcccccctcccactccccatgctgtccccagctcaccctacatggacttggcgatgtccttccatggctcaccggtctgaatttccatgatgagccgggcctgcagctttgctcccctatccctgcccaggctgcagctgtccatgcagggagcgagctccagcacctgcggagtccttccgtgggggcctctccgtgccacagcagccagggacctcaggtgcctgtgcatgacaccaccgcccatcctcatcctgagccagcctctcaggatcaggacttggtttggcggcgttaaccttagagcctgcaaggggcttcctcctggtgggtctggccgtagcctggggaggccacagctccaggccactccagacctcccttcctctgggccttccatgtggtggcaaccaccgcagctgtaagggagggaaaatggagcgtttgttctcgggctgggctggggtctgggggaagccatgggcgtgaagactggagtattatttgatggagaagcggccactcctggagaccggcggcaaacacagaagcacagcgtggaaggtgctggtgtcagcccacacgggtgatggggtcagactcaggagtcacactcaggagtcaccaggctcaaagggcccaggcaccgcaagtcctgctcagccccagacacaatgcattcctgttgccctcgccctcagccaggccccacgcaggccagggagcactggcaaagcttggcaaccctctgggggccagccttcatccaggccgaaggtggtcagtggcccaccatggcccaggtagaaaactcacggattaagatttcatgcccgactccaaaggcaagagactttattattttattttttttgagccagagtatcgctctgtcacctaggctggagtgcaatctctgctcattgcaacatctgcctcccgaactcaagcaattctgcctcagcctcccaagtagctgggattacaggtgtgcgccaccatgcccaggtaattgtatttttagtagagacagggtttcaccatgttggtcaggctggtttcaaactcctgacctcaaatgatctgcccacctcgacctcccaaagtgctgggattacaggtgcgagccaccgcacctggctaccagacacttcagagttacaggttagtttttctttttcttttatttttttttttttggcggaggtgcagggggagttaaacaaacaaacaaaataaacaggccgggtgcggtggctcatgcctgtaatcccagcactttaggaggcctaggtgggtggatcacgagatcaggggttcaagaccagcctggccgagatggtaaaaccccgtctccactaaaaatacaaaaattggccaggcacggtggctcacacctgtaatcccagtactttgggaggctgaggtgggcagatcacctgaggtcaggagttcaagaccaacctgaccaacatggagaaaccccatctctactaaaaatacaaaattagccaggtgtggtggtgcatgcctgtaattccagctactcgggaggctgaggcaggagaattgcttgaacccaggaggcagaggttgcagtgggccaagatggcgccattgcactccagcctgggaacaagagcgaaactctgactaaaaaagaaagaaagaaagaaaaaaattagttgggcacggtggcaggcgcctgtaatcccaggtactcaggaggctgaggcaggagaattgcttgaacccgggaggcagaggtcgcagtgagccgagattgcaccactgccctccagcctgggtgacagagcaagactccgtctcaaaaaaaaaaaaaaaaaaaattggatacattgtaatacctcaaatacttgtaagtgaagcaccccagttcccatagagctgccgcactcagaggcttctgtaacctgcctgctcccagcattctatttagggtctggtatgtccagaatttgcagacacagcaattcctgcagcagcagtgcaccatgtggaaggggccccatgaccagcccactgtgagctcacacgtgatgactgaggcttcttcacacagcagggctctgggtgtgatacccagggcacacgcgtttgcacaggcacaggccacacaagttctcacatgctcagccccataagccgtgctggacaggcatggccatttacacccaggatcctgctgagaacagcaaccaactcaccaccctcgcatcatgatccttgccacacaggggctctggtggctttggtggcctgggctgtggctctgctgccagccaccttgagtgaagatccgggttctctgggtgctactcagctgctatgtggggagctggcccctggggtgatgagggcccttcccaacccgccctcagcccttggacagccaggatcacccggggctgtctgcatacagacttctcaggggagttctcagcttggacccttatctccccagaatcctggaacctgctccttctgctctcgtgactgactgtgttctctatgcaacttccaataaaacctcttcatttgaaaggaaaaaa

SEQ ID NO: 110 - DNA, 909 bp, Homo sapiens
  source 1..909: mol_type = genomic DNA
atgcgtctgggttcacctggtctgctgtttctgctgttttcaagtctgcgtgctgatactcaggagaaggaagtccgggctatggtcggaagtgacgtggagctgtcatgcgcttgtcccgaagggtcccggttcgacctgaacgatgtctacgtgtattggcagacctctgagagtaagaccgtggtcacataccacatccctcagaactccagcctggaaaatgtggattcaaggtatcggaacagagccctgatgtcccctgctggcatgctgcggggagacttctctctgagactgtttaatgtgacaccacaggatgagcagaaattccattgcctggtcctgtcacagtccctgggatttcaggaggtgctgagtgtcgaagtgactctgcacgtcgccgctaatttctccgtgcctgtggtcagcgcaccacatagcccctctcaggacgagctgacctttacatgtacttccatcaacggctacccccgccctaacgtgtactggattaacaagactgacaatagcctgctggatcaggcactgcagaacgacaccgtgtttctgaatatgcgaggactgtacgatgtggtcagcgtcctgcgtattgccaggaccccatctgtgaacatcgggtgctgtattgagaacgtcctgctgcagcagaatctgacagtggggagccagactggtaatgacatcggcgagagggataagattaccgaaaaccccgtgagtacaggcgagaagaacgcagccacatggtcaatcctggctgtgctgtgcctgctggtggtcgtggctgtcgcaattggctgggtgtgccgcgatcggtgtctgcagcactcttatgccggtgcttgggcagtgagtccagagactgaactgaccggccatgtctaa

SEQ ID NO: 111 - protein, 322 aa, Mus musculus
MQLKCPCFVSLGTRQPVWKKLHVSSGFFSGLGLFLLLLSSLCAASAETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKVLVPVLAVLAAAAFVSFIIYRRTRPHRSYTGPKTVQLELTDHA

SEQ ID NO: 112 - DNA, 969 bp, Mus musculus
  source 1..969: mol_type = genomic DNA
atgcagctgaagtgtccatgcttcgtgtccctgggaacaagacagcccgtctggaagaaactgcacgtgagctccggcttctttagcggcctggggctgtttctgctgctgctgtctagtctgtgcgccgcttccgcagagactgaagtcggagccatggtgggcagtaacgtggtcctgtcatgcatcgacccacaccgacggcatttcaacctgtctggcctgtacgtgtattggcagattgagaatcccgaagtgtcagtcacctactatctgccttacaagagcccagggatcaacgtggactcaagctataaaaatagggggcacctgtccctggattctatgaagcagggaaacttcagcctgtacctgaaaaatgtgacccctcaggacacacaggagttcacttgtcgcgtctttatgaacactgcaaccgaactggtgaagattctggaggaagtggtccggctgagagtcgcagccaactttagcactcctgtgatctctaccagtgattcctctaatccaggccaggagcggacatatacttgcatgtctaagaacggataccccgaacctaatctgtattggatcaacaccacagacaatagtctgattgataccgctctgcagaacaatacagtctacctgaacaagctggggctgtatgacgtgatctctactctgcggctgccatggaccagtagaggagatgtgctgtgctgcgtggagaacgtggccctgcaccagaatatcacctcaattagccaggctgagtcctttaccggcaacaatacaaagaatcctcaggagacacataacaatgaactgaaagtgctggtgccagtgctggccgtcctggctgcagcagctttcgtgtcttttatcatctacagaaggacccgccctcaccgctcatacactggacctaagaccgtgcagctggaactgacagaccatgcttga

SEQ ID NO: 113 - skipped

SEQ ID NO: 114 - DNA, 1484 bp, Homo sapiens
  source 1..1484: mol_type = genomic DNA
cttaagatggaaactgacaccctgctgctgtgggtcctgctgctgtgggtgcctggatccaccggcgatatcgtgctgacccagtctcctggcacactgagtctgtcaccaggggagcgagcaacactgtcttgtagagccagccagtctgtgggaagctcctacctggcttggtatcagcagaagccaggccaggcacccaggctgctgatctacggagccttcagccgggccactggcattccagacaggttctctggaagtggctcagggaccgacttcaccctgaccatcagccgactggagcccgaagacttcgccgtgtactattgccagcagtacggctctagtccttggacttttggacagggcaccaaagtggagatcaagcgcggcgggggaggctctgggggaggcgggagtggaggcgggggatcacaggtccagctggtggaaagcggcgggggagtggtccagccaggccggagcctgcggctgagctgcgccgcttcaggattcacattttcaagctataccatgcactgggtccggcaggcaccagggaagggactggagtgggtgaccttcatcagctatgacggcaacaacaagtattacgctgattccgtgaaagggaggtttaccattagccgcgacaactccaaaaatacactgtacctgcagatgaacagcctgcgggccgaggatactgctatctactattgcgcaagaaccgggtggctgggacccttcgactattggggccaggggactctggtcaccgtgtcctctgataagacacacacatgccctccctgtcctgcaccagagctgctgggcgggccatccgtgttcctgtttccacccaagcctaaagacaccctgatgatcagccggacacctgaagtcacttgcgtggtcgtggacgtgagtcacgaggatccagaagtcaagtttaactggtacgtggatggcgtcgaggtgcataatgccaagaccaaacctcgcgaggaacagtacaatagcacatatcgagtcgtgtccgtcctgactgtgctgcatcaggattggctgaacggcaaagagtataagtgcaaagtgagcaataaggcactgcctgccccaatcgagaaaacaatttccaaggctaaaggccagcccagggaacctcaggtgtacactctgcctccaagtcgcgaggaaatgaccaagaaccaggtgagcctgacctgtctggtgaaagggttctatccatcagacattgcagtggagtgggaaagcaatggacagcccgaaaacaattacaagaccacaccccctgtgctggacagcgatggctccttctttctgtattctaagctgactgtggataaaagtcgctggcagcaggggaacgtctttagctgttccgtgatgcatgaggctctgcacaatcattacacacagaagtctctgagtctgtcacccggcaaatgaggcgcgcc

SEQ ID NO: 115 - skipped

SEQ ID NO: 116 - DNA, 1574 bp, Mus musculus
  source 1..1574: mol_type = genomic DNA
cttaagatggaaactgatactctgctgctctgggtgctgctcctctgggtgcctggttcaactggggacattcgacgggctgacattgtgatgacccagaccacactgagcctgcccgtgtccctgggcgaccaggccagcatctcctgccggagctcccagtctatcgtgcacagcaacggaaacacatacctggagtggtatctgcagaagcctggccagtccccaaagctgctgatctacaaggtgtccaacaggttcagcggcgtgcctgaccgcttttctggaagcggctccggaacagatttcaccctgaagatcagcagggtggaggctgaggacctgggcgtgtactactgcttccagggatcccacgtgccttacacctttggcggaggcacaaagctggagatcaagagagccgatgctgctccaaccgtgtctggaagcggaggcgggggttctggaggcggtgggagcggtggcggagggtctgaggctaagctgcaggagagcggccccgtgctggtgaagcctggagccagcgtgaagatgtcctgtaaggcttctggatacaccttcacagactactacatgaactgggtgaagcagagccacggcaagtccctggagtggatcggagtgatcaacccttacaacggcgacacctcttacaaccagaagtttaagggcaaggccaccctgacagtggataagtctagctccaccgcttacatggagctgaacagcctgacatccgaggattctgccgtgtactactgtgctaggtactacggaagctggttcgcctactggggccagggaacactgatcaccgtgtccacagccaagaccacaccccctagcgtgtaccccctggctcctaggtctagcagaggctgcaagccatgcatctgtaccgtgcccgaggtgagcagcgtgttcatctttccacccaagcccaaggacgtgctgaccatcacactgacccctaaggtgacatgcgtggtggtggatatcagcaaggacgatccagaggtgcagttctcctggtttgtggacgatgtggaggtgcacaccgcccagacacagccaagggaggagcagttcaactccacctttagatccgtgtctgagctgcccatcatgcaccaggactggctgaacggaaaggagttcaagtgccgggtgaactccgccgcttttcctgctccaatcgagaagaccatctctaagacaaagggccgcccaaaggctccacaggtgtacaccatccctccacccaaggagcagatggctaaggataaggtgagcctgacctgtatgatcacagacttctttcccgaggatatcacagtggagtggcagtggaacggacagcctgccgagaactacaagaacacccagccaatcatggacacagatggctcttacttcgtgtacagcaagctgaacgtgcagaagtctaactgggaggctggcaacaccttcacctgcagcgtgctgcacgaaggtctccataatcaccacaccgaaaagagcctcagtcacagccctgggaaatgaggcgcgcc

SEQ ID NO: 117 - DNA, 632 bp, synthetic construct
  source 1..632: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatt

SEQ ID NO: 118 - DNA, 394 bp, synthetic construct
  source 1..394: mol_type = other DNA
tgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctcc

SEQ ID NO: 119 - DNA, 188 bp, synthetic construct
  source 1..188: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagac

SEQ ID NO: 120 - DNA, 249 bp, synthetic construct
  source 1..249: mol_type = other DNA
gacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 121 - DNA, 345 bp, synthetic construct
  source 1..345: mol_type = other DNA
gctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagct

SEQ ID NO: 122 - DNA, 99 bp, synthetic construct
  source 1..99: mol_type = other DNA
gacctctggctaataaaggaaatttattttcattgcaatagtgtgttggaattttttgtgtctctcactcggaaggacatatgggagggcaaatcattt

SEQ ID NO: 123 - DNA, 720 bp, synthetic construct
  source 1..720: mol_type = other DNA
accatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 124 - DNA, 454 bp, synthetic construct
  source 1..454: mol_type = other DNA
ttaattaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgcttaag

SEQ ID NO: 125 - DNA, 1349 bp, synthetic construct
  source 1..1349: mol_type = other DNA
ttaattaagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcggttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttaag

SEQ ID NO: 126 - DNA, 481 bp, synthetic construct
  source 1..481: mol_type = other DNA
gacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttt

SEQ ID NO: 127 - protein, 10 aa, Homo sapiens
EAAGIGILTV

SEQ ID NO: 128 - protein, 10 aa, Mus musculus
EAAGIGILIV